(19) United States
(12) Patent Application Publication
(10) Pub. No.: US 2004/0116489 A1
(43) Pub. Date: Jun. 17, 2004

(54) Synthetic excitatory amino acids

(76) Inventors: Steven Marc Massey, Indianapolis, IN (US); James Allen Monn, Indianapolis, IN (US); Matthew John Valli, Zionsville, IN (US); Lourdes Prieto, Madrid (ES)

Correspondence Address: ELI LILLY AND COMPANY, PATENT DIVISION, P.O. BOX 6288, INDIANAPOLIS, IN, 46206-6288, US

(21) Appl. No.: 10/467,429

(22) PCT Filed: Feb. 12, 2002
(86) PCT No.: PCT/US02/01247

(51) Int. Cl. A61K 31/423; A61K 31/41; A61K 31/325; A61K 31/198

(52) U.S. Cl. 514/376; 514/381; 514/484; 514/566

(57) ABSTRACT

The present invention provides novel compounds that affect certain excitatory amino acid receptors, and are useful in the treatment of neurological disorders and psychiatric disorders.

### BACKGROUND OF THE INVENTION

In the mammalian central nervous system (CNS), the transmission of nerve impulses is controlled by the interaction between a neurotransmitter, that is released by a sending neuron, and a surface receptor on a receiving neuron, causing excitation of this receiving neuron. L-Glutamate, which is the most abundant neurotransmitter in the CNS, mediates the major excitatory pathway in mammals, and is referred to as an excitatory amino acid (EAA). The receptors that respond to glutamate are called excitatory amino acid receptors (EAA receptors). See Watkins & Evans, Ann. Rev. Pharmacol. Toxicol., 21, 165 (1981); Monaghan, Bridges, and Cotman, Ann. Rev. Pharmacol. Toxicol., 29, 365 (1989); Watkins, Krogsgaard-Larsen, and Honore, Trans. Pharm. Sci., 11, 25 (1990). The excitatory amino acids are of great physiological importance, playing a role in a variety of physiological processes, such as long-term potentiation (learning and memory), the development of synaptic plasticity, motor control, respiration, cardiovascular regulation, emotional states and sensory perception.

The excessive or inappropriate stimulation of excitatory amino acid receptors leads to neuronal cell damage or loss by way of a mechanism known as excitotoxicity. This process has been suggested to mediate neuronal degeneration in a variety of conditions. The medical consequences of such neuronal degeneration make the abatement of these degenerative neurological processes an important therapeutic goal.

Excitatory amino acid receptors are classified into two general types. Receptors that are directly coupled to the opening of cation channels in the cell membrane of the neurons are termed “ionotropic.” This type of receptor has been subdivided into at least three subtypes, which are defined by the depolarizing actions of the selective agonists N-methyl-D-aspartate (NMDA), α-amino-3-hydroxy-5-methylisoxazole-4-propionic acid (AMPA), and kainic acid (KA). The second general type of receptor is the G-protein or second messenger-linked “metabotropic” excitatory amino acid receptor. This second type is coupled to multiple second messenger systems that lead to enhanced phosphoinositide hydrolysis, activation of phospholipase D, increases or decreases in cAMP formation, and changes in ion channel function. Schoepp and Conn, Trends in Pharmacol. Sci., 14, 13 (1993). Both types of receptors appear not only to mediate normal synaptic transmission along excitatory pathways, but also participate in the modification of synaptic connections during development and throughout life. Schoepp, Rockaert, and Sladeczek, Trends in Pharmacol. Sci., 11, 508 (1990); McDonald and Johnson, Brain Research Reviews, 15, 41 (1990).

The metabotropic glutamate receptors are a highly heterogeneous family of glutamate receptors that are linked to multiple second-messenger pathways. Generally, these receptors function to modulate the presynaptic release of glutamate, and the postsynaptic sensitivity of the neuronal cell to glutamate excitation. The metabotropic glutamate receptors (mGluR) have been pharmacologically divided into two subtypes. One group of receptors is positively coupled to phospholipase C, which causes hydrolysis of cellular phosphoinositides (PI). This first group are termed PI-linked metabotropic glutamate receptors. The second group of receptors is negatively coupled to adenyl cyclase, which prevents the forskolin-stimulated accumulation of cyclic adenosine monophosphate (cAMP). Schoepp and Conn, Trends Pharmacol. Sci., 14, 13 (1993). Receptors within this second group are termed cAMP-linked metabotropic glutamate receptors. Agonists of the cAMP-linked metabotropic glutamate receptors should be useful for the treatment of acute and chronic neurological conditions and psychiatric conditions.

Compounds have recently been discovered that affect metabotropic glutamate receptors, but have no effect on ionotropic glutamate receptors. (1S,3R)-1-Aminocyclo-pentane-1,3-dicarboxylic acid (1S,3R-ACPD) is an agonist of PI-linked and cAMP-linked metabotropic glutamate receptors. Schoepp, Johnson, True, and Monn, Eur. J. Pharmacol., 207, 351 (1991); Schoepp, Johnson, and Monn, J. Neurochem., 58, 1184 (1992). (2S,3S,4S)-2-(carboxycyclopropyl)glycine (L-CCG-I) was recently described as a selective cAMP-linked metabotropic glutamate receptor agonist; however, at higher concentrations, this compound has activity at PI-linked metabotropic receptors. Nakagawa, et al., Eur. J. Pharmacol., 184, 205 (1990); Hayashi, et al., Br. J. Pharmacol., 107, 539 (1992); Schoepp et al., J. Neurochem., 63., page 769-772 (1994).

U.S. Pat. No. 5,958,960 discloses that certain 2-aminobicyclo[3.1.0]hexane-2,6-dicarboxylic acid derivatives are modulators of metabotropic glutamate function, in particular agonists or antagonists of glutamate at metabotropic glutamate receptors and as such are useful in the treatment of a neurological disorder or a psychartic disorder that has been linked to the excitatory amino acid receptors.

### SUMMARY OF THE INVENTION

The present invention provides compounds that selectively affect the negatively coupled cAMP-linked metabotropic glutamate receptors. More specifically, the present invention relates to compounds of the formula

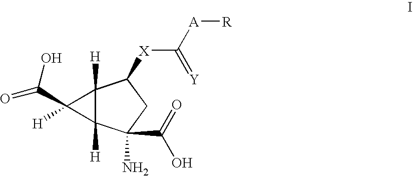

wherein:

X is CH₂, O, or NH;

Y is O, S, N or H;

A is a bond, O, N, (1-10C) alkyl, (2-10C) alkenyl or (2-10C) alkynyl;

R is hydrogen, (1-10C) akyl, (2-10C) alkenyl, (3-6C) alkynyl, aryl, heterocyclyl or substituted aryl;

or the group XC(Y)AR is

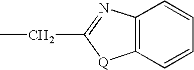

where Q is O, S or NH;

or a pharmaceutically acceptable metabolically labile ester or amide thereof;

or a pharmaceutically acceptable salt thereof.

A particular compound of formula I is one wherein (1-10C) alkyl is methyl, ethyl, n-propyl, isopropyl, n-butyl, t-butyl, n-pentyl, isopentyl, n-hexyl, heptyl, n-octyl, nonyl or decyl; (2-10C) alkenyl is allyl, allenyl, 1-butenyl, 1-pentenyl, 3-nonenyl or 5-decenyl; (2-6C) alkynyl is ethynyl, propynyl, butynyl or pentynyl; aryl is phenyl, substituted phenyl or naphthyl; and arylalkyl is benzyl, 2-nitro benzyl, or 1-phenylethyl.

Another particular compound of formula I is one wherein R is 2-methoxyphenyl, 3-methoxyphenyl, 4-methoxyphenyl, 3-methylphenyl, 4-methylphenyl, 2-chlorophenyl, 3-chlorophenyl, 4-chlorophenyl, 3-fluorophenyl, 2-fluorophenyl, 4-fluorophenyl, 2,6-dichlorophenyl, 2,4-dichlorophenyl, 3,5-dichlorophenyl, 3,4-dichlorophenyl, methyl, 2-napthyl, 1-napthyl, 3-methylphenyl, phenyl, thiophenyl, 3-trifluoromethylphenyl, 2,3dichlorophenyl, 1H-indole-3-yl cyclopropanyl, 1H-indol-2-yl, 4-hydroxyphenyl, 3-hydroxyphenyl, 2-hydroxyphenyl, 4-biphenylyl, 1-isoquinolyl, 3-pyridinyl, 2-pyridinyl, 3,5-difluorophenyl, 4-pyridinyl, 2-methylphenyl, 2-quinoxalinyl, hydrogen, 3-carboxyphenyl, 2-trifluoromethylphenyl, benzyl, 4-trifluoromethylphenyl;

A is a bond, methyl, O, NH, ethenyl or ethynyl;

Y is O, S or H,H; and

X is CH₂, O or NH.

Another particular compound of formula I is one wherein R is heterocyclyl or substituted aryl.

Another particular compound of formula I is one wherein R is 1H-indolyl, 2-napthyl, 3-chlorophenyl or 2-methoxyphenyl.

Another particular compound of formula I is one wherein Y is O or S.

Another particular compound of formula I is one wherein A is CH₂ or a bond.

A preferred compund of formula I is one wherein (1S*, 2S*, 4S*, 5R*, 6S*) 2-amino-4-[3-3-chlorophenyl)ureido]bicyclo[3.1.0]hexane-2,6-dicarboxylic acid.

The compounds of formula I are modulators of metabotropic glutamate receptor function with improved activity, in particular potent agonists of mGluR2 and mGluR3 receptors.

According to another aspect, therefore, the present invention provides a method of modulating metabotropic glutamate receptor function in a mammal including a human, which comprises administering an effective amount of a compound of formula I, or a non-toxic metabolically labile ester or amide thereof, or a pharmaceutically acceptable salt thereof.

According to yet another aspect, the present invention provides the use of a compound of formula I as defined hereinabove for the manufacture of a medicament for use in modulating metabotropic glutamate receptor function.

The present invention also includes all physical forms of the compounds of formula I, including crystalline solvates.

It will be appreciated that the compounds of formula I contain at least five asymmetric carbon atoms. As used herein, a wedge line refers to a bond projecting from the plain of the paper to the reader's eyes and is referred to as “beta” stereochemistry. A dashed line refers to a bond projecting into the plain of the paper away from the reader's eyes and is referred to as “alpha” stereochemistry. A plain line refers to a bond lying within the plain of the paper. Preferably the stereoisomer of compounds of formula I have the configuration I with functional groups attached at C-4 possesing beta stereochemistry, as shown below.

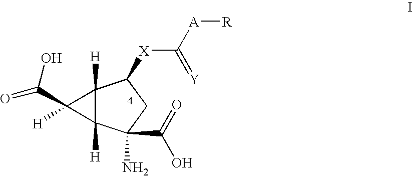

The present invention also provides pharmaceutical formulations comprising a compound of formula I in combination with one or more pharmaceutically acceptable carriers, diluents, or excipients.

Further aspects of the present invention include a method for affecting the cAMP-linked metabotropic glutamate receptors, as well as methods for treating a neurological disorder or a psychiatric disorder that has been linked to the excitatory amino acid receptors, which comprises administering a compound of formula I. Examples of neurological disorders that are treated with a formula I compound include cerebral deficits subsequent to cardiac bypass surgery and grafting, cerebral ischemia (e.g. stroke and cardiac arrest); spinal cord trauma; head trauma; Alzheimer's Disease; Huntington's Chorea; amyotrophic lateral sclerosis; AIDS-induced dementia; muscular spasms; migraine headaches; urinary incontinence; convulsions; perinatal hypoxia; hypoglycemic neuronal damage; drug tolerance, withdrawal, and cessation (i.e. opiates, benzodiazepines, nicotine, cocaine, or ethanol); smoking cessation; ocular damage and retinopathy; cognitive disorders; idiopathic and drug-induced Parkinson's Disease; emesis; brain edema; chronic pain; sleep disorders; Tourette's syndrome; attention deficit disorder; and tardive dyskinesia. Examples of psychiatric disorders that are treated with a formula I compound include schizophrenia, anxiety and related disorders (e.g. panic attack and stress-related disorders), depression, bipolar disorders, psychosis, and obsessive compulsive disorders.

The present invention also provides a process for producing a compound of formula I, or a pharmaceutically salt thereof, which comprises:

(1) deprotecting the amine group of a compound of formula

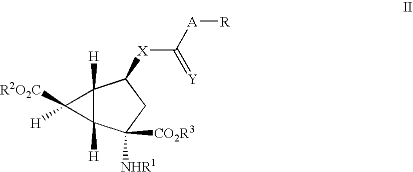

wherein:

R¹ is a nitrogen protecting group such t-butoxycarbonyl; and

R² and R³ are both carboxy protecting groups such as ethyl;

(2) for a compound of formula I deprotecting the carboxy groups of a compound of formula II where R²and R³ are both carboxy protecting groups such as ethyl;

(3) optionally preparing a pharmaceutically-acceptable salt of the formula I compound.

### DETAILED DESCRIPTION OF THE INVENTION

The term “(1-10C) alkyl” represents a straight, branched, or cyclic alkyl chain having from one to ten carbon atoms. Typical straight or branched C₁-C₁₀ alkyl groups include methyl, ethyl, n-propyl, isopropyl, n-butyl, isobutyl, sec-butyl, t-butyl, n-pentyl, isopentyl, neopentyl, n-hexyl, 2-methylpentyl, 3-methylpentyl, 4-methylpentyl, 2,2-dimethylbutyl, 2,3-dimethylbutyl, 3,3-dimethylbutyl, heptyl, n-octyl, 2,2-dimethylhexyl, 2,5-dimethylhexyl, 2-methylheptyl, 4-methylheptyl, 2,2,4-trimethylpentyl, 2,3,4-trimethylpentyl, nonyl, 3,5,5-trimethylhexyl, decyl, 3,7-dimethyloctyl, and the like. The term “(1-10C) alkyl” includes within it the terms “C₁-C₆ alkyl” and “C₁-C₄ alkyl”. Typical cyclic alkyl groups include cyclopropyl, cyclobutyl, cyclopentyl, cyclohexyl, cycloheptyl, and the like. Typical C₁-C₆ alkyl groups include methyl, ethyl, n-propyl, isopropyl, n-butyl, isobutyl, sec-butyl, t-butyl, n-pentyl, isopentyl, neopentyl, and n-hexyl.

The term “(2-10C) alkenyl” represents straight or branched unsaturated alkyl chains having from two to ten carbon atoms, and having one or more carbon-carbon double bond, such as, dienes and trienes. This group also includes both E and Z isomers. Representative radicals for this group include ethenyl, allyl, allenyl, 1-butenyl, 2-butenyl, 2-methyl-1-propenyl, 3-butenyl, 2-methyl-2-propenyl, butadienyl, 1-pentenyl, 2-pentenyl, 2-methyl-2-butenyl, 4-pentenyl, 3-methyl-2-butenyl, 3-methyl-1,2-butadienyl, 3-hexenyl, 2-hexenyl, 4-methyl-3-pentenyl, 4-hexenyl, 5-hexenyl, 3-methyl-1-penten-3-yl, 4-methyl-3-pentenyl, 6-methyl-5-heptene-2-yl, 7-octenyl, 1-octen-3-yl, 3-nonenyl, 2,4-dimethyl-2,6-heptadienyl, 3,7-dimethyl-6-octenyl, 5-decenyl, 9-decenyl, 2,6-dimethyl-7-octenyl, and the like. The term “(2-10C) alkenyl” includes within it the term “(2-6C) alkenyl”.

The term “(2-6C) alkynyl” represents ethynyl, propynyl, butynyl or pentynyl.

The phrase “stereoisomeric compound” represents an optical isomer of a Formula I compound, which includes the 1S, 2S, 4S, 5R, 6R isomer.

The term “carboxy protecting group” as used herein refers to one of the ester derivatives of the carboxylic acid group commonly employed to block or protect the carboxylic acid group while reactions are carried out on other functional groups. The protection of carboxylic acid groups is generally described in McOmie, Protecting Groups in Organic Chemistry, Plenum Press, NY, 1973; and Greene and Wuts, Protecting Groups in Organic Synthesis, 2nd. Ed., John Wiley & Sons, NY, 1991. Examples of such carboxy protecting groups include methyl, ethyl, methoxymethyl, methylthiomethyl, triphenylmethyl, benzyl, 4-nitrobenzyl, 4-methoxybenzyl, 3,4-dimethoxy-benzyl, 2,4-dimethoxybenzyl, 2,4,6-trimethoxybenzyl, 2,4,6-trimethylbenzyl, benzhydryl, t-butyl, t-amyl, trityl, trimethylsilyl, t-butyldimethyl-silyl, allyl, 1-(trimethylsilylmethyl)-prop-1-en-3-yl, and the like. Particularly preferred carboxy protecting groups are (C₁-C₆) alkyl groups such as ethyl. The term “protected carboxy” refers to a carboxylic acid group having a carboxy protecting group.

The term “nitrogen protecting group” as used herein refers to substituents on amino groups that are commonly employed to block or protect the amino functionality while reactions are carried out in other functional groups. The protection of amino groups is generally described in McOmie, Protecting Groups in Organic Chemistry; Plenum Press, NY, 1973, and Greene and Wuts, Protecting Groups in Organic Synthesis, 2nd. Ed., John Wiley & Sons, NY, 1991. Examples of nitrogen protecting groups include benzyl, t-butyl, allyl, triphenylmethyl, t-butyldimethylsilyl, triphenylsilyl, formyl, trityl, phthalimido, trichloroacetyl, chloroacetyl, phthaloyl, 2-nitrophenoxyacetyl, benzyloxycarbonyl, methoxycarbonyl, 2-methylbenzyloxycarbonyl, t-butoxycarbonyl, allyloxy-carbonyl, 2,2,2-trichloroethoxycarbonyl, and the like. The term “protected amino” refers to a primary or secondary amine having a nitrogen protecting group.

The term “heterocyclyl” includes heteroaromatics an aromatic 5-6 membered ring containing from one to four heteroatoms selected from oxygen, sulfur and nitrogen, and a bicyclic group consisting of a 5-6 membered ring containing from one to four heteroatoms selected from oxygen, sulfur and nitrogen fused with a benzene ring or a 5-6 membered ring containing from one to four heteroatoms selected from oxygen, sulfur and nitrogen. Examples of heteroaromatic groups are furyl, thiophenyl, oxazolyl, isoxazolyl, thiazoyl, isothiazolyl, imidazolyl, pyrimidyl, benzofuryl, benzothiophenyl, benzimidazolyl, benzoxazolyl, benzo-thiazolyl and indolyl. Examples of particular values are 2-thienyl, 3-thienyl, 2-indolyl, 3-indolyl, 2-pyridyl, 3-pyridyl, 4-pyridyl, 1-isoquinolinyl, 3-isoquinolinyl, 2-benzothiazolyl, benzoxazoyl and 4-imidazolyl.

The term aryl includes phenyl and a polycyclic aromatic carbocyclic ring such as 1-naphthyl or 2-naphthyl.

The term “substituted”, as used in the term “substituted heterocycles or aromatic group”, herein signifies that one, two or more substituents may be present, said substituents being selected from atoms and groups which, when present in the compound of formula I, do not prevent the compound of formula I from functioning as a modulator of metabotropic glutamate receptor function.

Examples of atoms and groups which may be present in an optionally substituted heteroaromatic or aryl group are amino, hydroxy, nitro, halogeno, (1-6C) alkyl, (1-6C) alkoxy, (1-6C) alkylthio, carboxy, (1-6C) alkoxycarbonyl, carbamoyl, (1-6C) alkanoylamino, (1-6C) alkylsulphonyl, (1-6C) alkylsulphonylamino, (1-6C)alkanoyl, phenyl, phenoxy, phenylthio, phenylsulphonyl, phenylsulphonylamino, toluene-sulphonylamino, and (1-6C)fluoroalkyl. Examples of particular values are hydroxy, fluoro, chloro, bromo, iodo, methyl, methoxy, carboxy, acetyl, phenyl, phenoxy, tetrazoyl and trifluoromethyl.

Examples of values for substituted aryl groups are 1-naphthyl, 2-naphthyl, phenyl, 2-hydroxyphenyl, 3-hydroxyphenyl, 4-hydroxyphenyl, 3,5-dihydroxyphenyl, 2-fluorophenyl, 3-fluorophenyl, 4-fluorophenyl 3,5-difluorophenyl, 2-chlorophenyl, 3-chlorophenyl, 4-chlorophenyl, 2,4-dichlorophenyl, 3,4-dichlorophenyl, 3,5-dichlorophenyl, 2-methylphenyl, 3-methylphenyl, 4-methylphenyl, 2-methoxyphenyl, 3-methoxyphenyl, 4-methoxyphenyl, 3-trifluoromethylphenyl, 4-trifluoromethylphenyl, 2-methylphenyl, 3-methylphenyl, 4-methylphenyl, 3-carboxyphenyl, 4-carboxyphenyl and 3-(5′-tetrazoyl)phenyl.

The term heterocyclyl also includes non-aromatic heterocyclyl which includes a 4 to 7 membered ring containing one or two heteroatoms selected from oxygen, sulphur and nitrogen, for example azetidin-1-yl or -2-yl, pyrrolidin-1-yl, -2-yl or -3-yl, piperidin-1-yl, -2-yl, -3-yl or -4-yl, hexahydroazepin-1-yl, -2-yl, -3-yl or -4-yl, oxetan-2-yl or -3-yl, tetrahydro-furan-2-yl or -3-yl, tetrahydropyran-2-yl, -3-yl or -4-yl, hexahydrooxepin-2-yl, -3-yl or -4-yl, thietan-2-yl or -3-yl, tetrahydrothiophen-2-yl or -3-yl, tetrahydrothiopyran-2-yl, -3-yl or -4-yl, hexahydrothiepin-2-yl, -3-yl or -4-yl, piperazin-1-yl or -2-yl, morpholin-1-yl, -2-yl or -3-yl, thiomorpholin-1-yl, -2-yl or -3-yl, tetrahydropyrimidin-1-yl, -2-yl, -4-yl or -S-yl, imidazolin-1-yl, -2-yl or -4-yl, imidazolidin-1-yl, -2-yl or -4-yl, oxazolin-2-yl, -3-yl, -4-yl or -5-yl, oxazolidin-2-yl, -3-yl, -4-yl or -5-yl, thiazolin-2-yl, -3-yl, -4-yl or -5-yl, or thiazolidin-2-yl, -3-yl, -4-yl or -5-yl.

The term “affecting” refers to a formula I compound acting as an agonist at an excitatory amino acid receptor. The term “excitatory amino acid receptor” refers to a metabotropic glutamate receptor, a receptor that is coupled to cellular effectors via GTP-binding proteins. The term “cAMP-linked metabotropic glutamate receptor” refers to a metabotropic receptor that is coupled to inhibition of adenylate cyclase activity.

The term “neurological disorder” refers to both acute and chronic neurodegenerative conditions, including cerebral deficits subsequent to cardiac bypass surgery and grafting, cerebral ischemia (for example stroke resulting from cardiac arrest), spinal cord trauma, head trauma, Alzheimer's Disease, Huntington's Chorea, amyotrophic lateral sclerosis, AIDS-induced dementia, perinatal hypoxia, hypoglycemic neuronal damage, ocular damage and retinopathy, cognitive disorders, idiopathic and drug-induced Parkinson's Disease. This term also includes other neurological conditions that are caused by glutamate dysfunction, including muscular spasms, migraine headaches, urinary incontinence, drug tolerance, withdrawal, and cessation (i.e. opiates, benzodiazepines, nicotine, cocaine, or ethanol), smoking cessation, emesis, brain edema, chronic pain, sleep disorders, convulsions, Tourette's syndrome, attention deficit disorder, and tardive dyskinesia.

The term “psychiatric disorder” refers to both acute and chronic psychiatric conditions, including schizophrenia, anxiety and related disorders (e.g. panic attack and stress-related cardiovascular disorders), depression, bipolar disorders, psychosis, and obsessive compulsive disorders.

As used herein the term “effective amount” refers to the amount or dose of the compound, upon single or multiple dose administration to the patient, which provides the desired effect in the patient under diagnosis or treatment.

An effective amount can be readily determined by the attending diagnostician, as one skilled in the art, by the use of known techniques and by observing results obtained under analogous circumstances. In determining the effective amount or dose of compound administered, a number of factors are considered by the attending diagnostician, including, but not limited to: the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the use of concomitant medication; and other relevant circumstances. For example, a typical daily dose may contain from about 150 micrograms to about 150 mg of the active ingredient. The compounds can be administered by a variety of routes including oral, rectal, transdermal, subcutaneous, intravenous, intramuscular, bucal or intranasal routes. Alternatively, the compound may be administered by continuous infusion.

As used herein the term “patient” refers to a mammal, such as a mouse, guinea pig, rat, dog or human. It is understood that the preferred patient is a human.

The term “treating” (or “treat”) as used herein includes its generally accepted meaning which encompasses prohibiting, preventing, restraining, and slowing, stopping, or reversing progression of a resultant symptom. As such, the methods of this invention encompass both therapeutic and prophylactic administration.

The present invention includes pharmaceutically-acceptable salts of the formula I compounds. These salts can exist in conjunction with the acidic or basic portion of the molecule and can exist as acid addition, primary, secondary, tertiary, or quaternary ammonium, alkali metal, or alkaline earth metal salts. Generally, the acid addition salts are prepared by the reaction of an acid with a compound of formula I.

Acids commonly employed to form such salts include inorganic acids such as hydrochloric, hydrobromic, hydriodic, sulfuric, and phosphoric acid, as well as organic acids such as para-toluenesulfonic, methanesulfonic, oxalic, para-bromophenylsulfonic, carbonic, succinic, citric, benzoic, and acetic acid, and related inorganic and organic acids. Such pharmaceutically-acceptable salts thus include sulfate, pyrosulfate, bisulfate, sulfite, bisulfite, phosphate, ammonium, monohydrogenphosphate, dihydrogenphosphate, meta-phosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, furmarate, hippurate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, α-hydroxybutyrate, glycolate, maleate, tartrate, methanesulfonate, propanesulfonate, naphthalene-1-sulfonate, napthalene-2-sulfonate, mandelate, magnesium, tetramethylammonium, potassium, trimethylammonium, sodium, methylammonium, calcium, and the like salts.

The absolute stereochemical configuration of this most preferred enantiomer has been determined to be 1S, 2S, 4S, 5R, 6S.

While all the formula I compounds of the present invention are believed to selectively affect the negatively-coupled cAMP-linked metabotropic glutamate receptors, certain compounds of the invention are preferred for such use. Preferably, X is NH, O or CH₂, Y is O, Z is NH or a bond, and R is aryl, heteroaryl, fused aryl or substituted aryl. Representative compounds from this preferred group of formula I compounds include (1S*, 2S*, 4S*, 5R*, 6S*) 2-amino-4-(3-chloro-benzoylamino)-bicyclo[3.1.0]hexane-2,6-dicarboxylic acid, (1S*, 2S*, 4S*, 5R*, 6S*) 2-amino-4-(3-methoxy-benzoylamino)-bicyclo[3.1.0]hexane-2,6-dicarboxylic acid, (1S*, 2S*, 4S*, 5R*, 6S*) 2-amino-4-(3-hydroxy-benzoylamino)-bicyclo[3.1.0]hexane-2,6-dicarboxylic acid, (1S*, 2S*, 4S*, 5R*, 6S*) 2-amino-4-(3,4-dichloro-benzoylamino)-bicyclo[3.1.0]hexane-2,6-dicarboxylic acid, (1S*, 2S*, 4S*, 5R*, 6S*) 2-amino-4-[(1H-indole-2-carbonyl)-amino]-bicyclo[3.1.0]hexane-2,6-dicarboxylic acid, (1S*, 2S*, 4S*, 5R*, 6S*) 2-amino-4-[(naphthalene-1-carbonyl)-amino]-bicyclo[3.1.0]hexane-2,6-dicarboxylic acid, (1S*, 2S*, 4S*, 5R*, 6S*) 2-amino-4-[(isoquinoline-1-carbonyl)-amino]-bicyclo[3.1.0]hexane-2,6-dicarboxylic acid, (1S*, 2S*, 4S*, 5R*, 6S*)-2-amino-4-(3-carboxy-benzoylamino)-bicyclo[3.1.0]hexane-2,6-dicarboxylic acid, (1S*,2S*,4S*,5R*,6S*) 2-amino-4-[3-(4-fluorophenyl)ureido]bicyclo[3.1.0]hexane-2,6-dicarboxylic acid, (1S*,2S*,4S*,5R*,6S*) 2-amino-4-[3-(3,5-dichlorophenyl)ureido)bicyclo[3.1.0]hexane-2,6-dicarboxylic acid, (1S*, 2S*, 4S*, 5R*, 6R*)-2-amino-4-(3-fluoro-phenylcarbamoyloxy)-bicyclo[3.1.0]hexane-2,6-dicarboxylic acid, and (1S*,2S*,4R*,5R*,6S*) 2-amino-4-(3-chloro)phenylcarbamoylmethyl bicyclo[3.1.0]hexane-2,6-dicarboxylic acid. Certain compounds of the present invention are more preferred for use in affecting the cAMP-linked metabotropic glutamate receptors. More preferably, R is heteroaromatic, aryl or substituted aryl. Representative compounds from this more preferred group of compounds include (1S*, 2S*, 4S*, 5R*, 6S*) 2-amino-4-[(1H-indole-3-carbonyl)-amino]-bicyclo[3.1.0]hexane-2,6-dicarboxylic acid, (1S*,2S*,4S*,5R*,6S*) 2-amino-4-[3-(2-naphthyl)ureido) bicyclo(3.1.0]hexane-2,6-dicarboxylic acid, (1S*, 2S*, 4S*, 5R*, 6R*)-2-amino-4-(3-chloro-phenylcarbamoyloxy)-bicyclo[3.1.0]hexane-2,6-dicarboxylic acid, and (1S*,2S*,4R*,5R*,6S*) 2-amino-4-(2-methoxy)-phenylcarbamoylmethyl bicyclo[3.1.0]hexane-2,6-dicarboxylic acid.

Most preferably, the compound of formula (I) is (1S*,2S*,4S*,5R*,6S*) 2-amino-4-[3-(3-chlorophenyl)ureido)bicyclo[3.1.0]hexane-2,6-dicarboxylic acid, a C₁-C₄ alkyl, aralkyl or aryl ester thereof or a pharmaceutically acceptable salt thereof.

While all the compounds of formula II of the present invention are believed to be useful for the synthesis of compounds of formula I, certain compounds are preferred. Preferably, R¹ is t-butoxycarbonyl. More preferably, R² and R³ are C₁-C₁₀ alkyl groups for example ethyl groups.

The compounds of formula I of the present invention are generally synthesized from compounds of formula III or formula X where R¹, R² and R³ are as previously described. The compounds of formula III and formula X are prepared as described in U. S. Patent No. 5,958,960 which is incorporated by reference in its entirety.

Generally, compounds of formula II in which X and Y are O, and A is N may be prepared by directly reacting compounds of formula III. Compounds of formula II in which X and A are N, and Y is 0 may be prepared by reacting compounds of formula IX. Compounds of formula IX may be prepared by reacting compounds of formula III in a series of steps to convert the hydroxyl to an amine while retaining the stereochemistry at point of attachment.

More specifically, compounds of formula III are reacted with sulphonating agents such as p-toluenesulphonyl chloride in a suitable solvent such as pyridine to produce compounds of formula IV. Compounds of formula IV are reacted with potassium nitrite in DMSO at around 50° C. to provide compounds of formula V. It will be appreciated that compounds of formula V have the hydroxyl in opposite configuration to that of the hydroxyl in compounds of formula III.

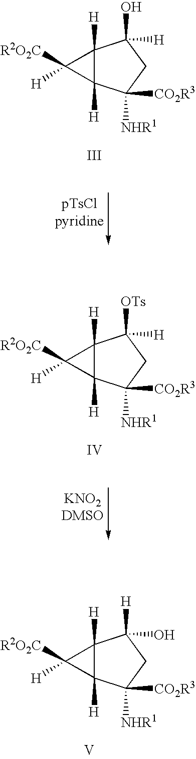

Compounds of formula V are reacted with sulphonating agents such as p-toluenesulphonyl chloride in a suitable solvent such as pyridine to produce compounds of formula VI. Compounds of formula VI are reacted with an azide salt such as sodium azide for example in dimethylsulfoxide as a reaction solvent to produce compounds of formula VII.

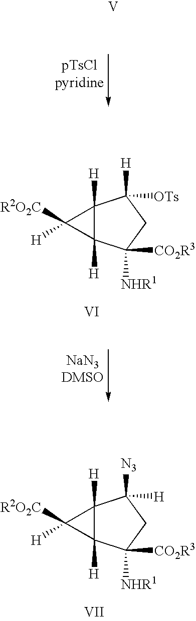

Alternatively, compounds of formula VII may be prepared by reacting compounds of formula III with halogenating agents such as bromine and triphenyl phosphine in the presence of a suitable base such as pyridine in a reaction solvent such as methylene chloride to produce compounds of formula VIII. Compounds of formula VIII are reacted with an azide salt such as sodium azide for example in dimethylsulfoxide as a reaction solvent to produce compounds of formula VII.

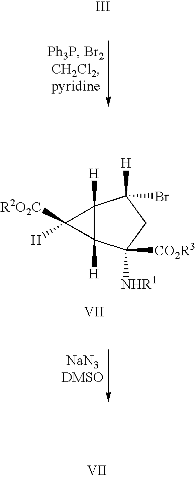

The compounds of formula IX may be prepared by reducing a corresponding compound of formula VII. The reduction is conveniently performed using triphenylphosphine in a suitable solvent such as tetrahydrofuran at a temperature in the range of 0 to 100° C.

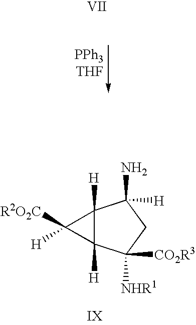

Compounds of formula II in which X and A are N, and Y is O may be prepared by reacting compounds of formula IX with an isocyanate of formula R—N═C═O. Convenient solvents include dichloromethane.

Compounds of formula II in which X and Y are O, and A is N may be prepared by reacting compounds of formula III with an isocyanate of formula R—N═C═O. Convenient solvents include dichloromethane.

Compounds of formula II in which X is N, Y is O and A is a bond may be prepared by reacting compounds of formula IX, an amine, with an acyl halide of formula RCOX¹ in which X¹ is, for example, chlorine or bromine. The reaction is conveniently performed in the presence of a base, such as diisopropylethyl amine and in a solvent such as dichloromethane. Alternatively, the amine may be reacted with an acyl isourea such as obtained by the reaction of a carboxylic acid of the formula RCOOH with a carbodiimide such as dicyclohexylcarbodiimide. The reaction is conveniently performed in the presence of an activating agent such as hydroxybenzotriazole and in a solvent such as dichloromethane.

Generally, compounds of formula II in which X is CH₂, Y is O, and A is N may be prepared by reacting, in a series of steps, compounds of formula X.

More specifically, compounds of formula XI where R⁴ is not hydrogen (a carboxy protecting group) may be prepared from compounds of formula X by a Wadsworth-Emmons reaction, for example by reaction with an alkali metal salt of a dialkyl phosphono acetate ester, such as the sodium salt of allyl diethylphosphonoacetate. The reaction is conveniently performed in an anhydrous solvent such as anhydrous THF. It is appreciated that compounds of formula XI may exist in the (E) or (Z) isomeric form or as a mixture of (E) and (Z) isomers and, as such, are included in the present invention.

Compounds of formula XI where R⁴ is hydrogen (a acid) may be prepared from compounds of formula XI where R⁴ is not hydrogen (a carboxy protecting group) by procedures well known in the art. In the case where R⁴ is allyl, compounds of formula XI are reacted with a metal catalyst such as chlorotristriphenylphosphine rohodium(I) or tetrakistriphenylphosphine palladium(O) to produce compounds of formula XI where R⁴ is hydrogen. The reaction is conveniently performed in the presence of a base such as pyrrolidine and in solvents such as ethanol/water or dichloromethane.

Compounds of formula XII may be prepared by reacting compounds of formula XI where R⁴ is hydrogen with oxalyl chloride and catalytic DMF in a suitable solvent such as dichloromethane to produce an intermediate acyl chloride. The intermediate acyl chlorides are reacted with excess amine such as aniline in a suitable solvent such as dichloromethane.

Compounds of formula II where X is CH₂ may be prepared by reacting compounds of formula XII with hydrogen in the presence of a suitable metal catalyst such as palladium on carbon. Convenient solvents include ethanol.

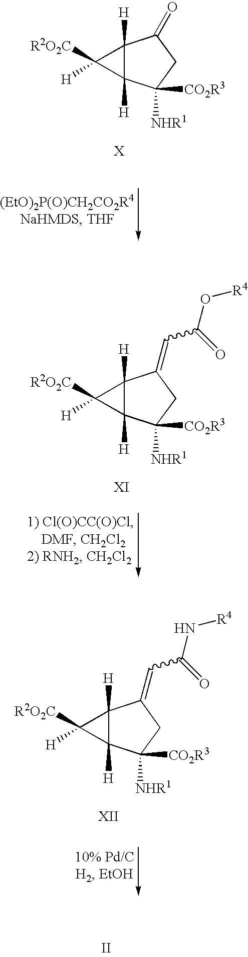

The formula I compounds of the present invention are agonists of certain metabotropic excitatory amino acid receptors. Specifically, the formula I compounds are agonists of the negatively-coupled cAMP-linked metabotropic glutamate receptors. Therefore, another aspect of the present invention is a method of affecting an excitatory amino acid receptor in mammals, which comprises administering to a mammal requiring modulated excitatory amino acid neurotransmission a pharmaceutically-effective amount of a compound of formula I. The term “pharmaceutically-effective amount” is used to represent an amount of the compound of the invention which is capable of affecting the excitatory amino acid receptors. By affecting, a compound of the invention is acting as an agonist. When a compound of the invention acts as an agonist, the interaction of the compound with the EAA receptor mimics the response of the interaction of this receptor with its natural ligand (i.e. L-glutamate).

The particular dose of compound administered according to this invention will of course be determined by the particular circumstances surrounding the case, including the compound administered, the route of administration, the particular condition being treated, and similar considerations. The compounds can be administered by a variety of routes including oral, rectal, transdermal, subcutaneous, intravenous, intramuscular, or intranasal routes. Alternatively, the compound may be administered by continuous infusion.

A variety of physiological functions have been shown to be subject to influence by excessive or inappropriate stimulation of excitatory amino acid transmission. The formula I compounds of the present invention are believed to have the ability to treat a variety of neurological disorders in mammals associated with this condition, including acute neurological disorders such as cerebral deficits subsequent to cardiac bypass surgery and grafting, cerebral ischemia (e.g. stroke and cardiac arrest), spinal cord trauma, head trauma, perinatal hypoxia, and hypoglycemic neuronal damage. The formula I compounds are believed to have the ability to treat a variety of chronic neurological disorders, such as Alzheimer's disease, Hungington's Chorea, amyotrophic lateral sclerosis, AIDS-induced dementia, ocular damage and retinopathy, cognitive disorders, and idopathic and drug-induced Parkinson's. The present invention also provides methods for treating these disorders which comprises administering to a patient in need thereof an effective amount of a compound of formula I.

The formula I compounds of the present invention are also believed to have the ability to treat a variety of other neurological disorders in mammals that are associated with glutamate dysfunction, including muscular spasms; convulsions; migraine headaches; urinary incontinence; psychosis; drug tolerance, withdrawal, and cessation (i.e. opiates, benzodiazepines, nicotine, cocaine, or ethanol); smoking cessation; anxiety and related disorders (e.g. panic attack and stress-related disorders); emesis; brain edema; chronic pain; sleep disorders; Tourette's syndrome; attention deficit disorder; and tardive dyskinesia. Therefore, the present invention also provides methods for treating these disorders which comprise administering to a patient in need thereof an effective amount of the compound of formula I.

The compounds of the present invention are agonists of cAMP-linked metabotropic glutamate receptors. These compounds are negatively coupled through the receptor to adenyl cyclase, inhibiting the formation of cyclic adenosine monophosphate. The formula I compounds of the present invention are, therefore, believed to have the ability to treat a variety of psychiatric disorders, such as schizophrenia, anxiety and related disorders (e.g. panic attack and stress-related disorders), depression, bipolar disorders, psychosis, and obsessive compulsive disorders. The present invention also provides methods for treating these disorders which comprises administering to a patient in need thereof an effective amount of a compound of formula I.

To study the ability to affect receptor binding of compounds of the present invention in comparison to compounds of U.S. Pat. No. 5,958,960, displacement of a high affinity mGluR2 antagonist radioligand [³H]LY341495 from recombinant human mGluR2 and human mGluR3 receptors expressed in RGT cells was determined. (See, Ornstein P. L., Arnold M. B., Bleisch T. J., Wright R. A., Wheeler W. J., and Schoepp D. D., [³H]LY341495, a highly potent, selective and novel radioligand for labeling group II metabotropic receptors. Bioorg. Med. Chem. Lett. 8: 1919-1922 (1998); and Johnson B. G., Wright R. A., Arnold M. B., Wheeler W. J., Ornstein P. L., and Schoepp D. D., [³H]LY341495 as a novel rapid filtration antagonist radioligand for group II metabotropic receptors: Characterization of binding to membranes of mGlu receptor subtype expressing cells. Neuropharmacology 38: 1519-1529 (1999)). As shown in table 1, compounds of the current invention have improved affinty for the mGlu3 receptors when compared to compounds of U.S. Pat. No. 5,958,960.

TABLE 1
Effects on [³H]LY341495 binding to mGlu2 and mGlu3
receptors.

Kᵢ (nM) ± SEM

| | mGlu2 | mGlu3 |
|---|---|---|
| Compounds of | | |
| U.S. Pat. No. 5,958,960 | | |
| (1S*, 2S*, 4R*, 5R*, 6S*) 2- | 468.3 ± 29.0 | 408.0 ± 25.4 |
| Amino-4-(3-methylureido) | | |
| bicyclo[3.1.0]hexane-2,6- | | |
| dicarboxylic acid | | |
| (1S*, 2S*, 4R*, 5R*, 6S*) | 638.1 ± 59.6 | 399.3 ± 67.8 |
| 4-Acetylamino-2-aminobicyclo | | |
| [3.1.0]hexane-2,6- | | |
| dicarboxylic acid | | |
| 1S*, 2S*, 4R*, 5R*, 6S*) 2- | 607.3 ± 10.2 | 393.4 ± 17.8 |
| Amino-4-benzoylaminobicyclo | | |
| [3.1.0]hexane-2,6- | | |
| dicarboxylic acid | | |
| Compounds of present | | |
| invention | | |
| (1S*, 2S*, 4S*, 5R*, 6S*) 2- | 254.2 ± 10.7 | 23.0 ± 2.6 |
| Amino-4-(3-methylureido) | | |
| bicyclo[3.1.0]hexane-2,6- | | |
| dicarboxylic acid | | |
| (1S*, 2S*, 4S*, 5R*, 6S*) | 772.1 ± 38.8 | 213.7 ± 14.6 |
| 4-Acetylamino-2-aminobicyclo | | |
| [3.1.0]hexane-2,6- | | |
| dicarboxylic acid | | |
| (1S*, 2S*, 4S*, 5R*, 6S*) 2- | 511.5 ± 89 | 9.54 ± 0.73 |
| Amino-4-benzoylaminobicyclo | | |
| [3.1.0]hexane-2,6- | | |
| dicarboxylic acid | | |

To study the functional effect of compounds of the present invention in comparison to compounds of U.S. Pat. No. 5,958,960, forskolin stimulated c-AMP production in cells expressing recombinant mGlu2 and mGlu3 receptors was determined. As mGlu2 and mGlu3 receptors are negatively coupled to adenyl cyclase, activity of an agonist decreases forskolin-stimulated accumulation of cyclic adenosine monophosphate (cAMP) and is reported as the concentration necessary to reduce C-AMP accumulation to 50% of the controls (EC₅₀, nM). The activity of an anatgonist blocks the agonist induced decrease in forskolin-stimulated accumulation of cyclic adenosine monophosphate (cAMP) and is reported as the concentration necessary to inhibit the reduction of C-AMP accumulation to 50% of the controls (IC₅₀, nM). (Schoepp D. D., Johnson B. G., Wright R. A., Salhofff C. R., Mayne N. G., Wu S., Cockerham S. L., Burnett J. P., Belagaje R., Bleakman D., and Monn J., LY354740 is a potent and highly selective group II metabotropic glutamate receptor agonist in cells expressing human glutamate receptors. Neuropharmacology 36: 1-11 (1997)). As shown in table 2, compounds of the current invention have agonist effects on cells expressing mGlu2 and mGlu3 receptors where as compounds of U.S. Pat. No. 5,958,960 have antagonist effects on cells expressing mGlu2 and mGlu3 receptors.

TABLE 2
Effects on forskolin-stimulated accumulation of
cyclic adenosine monophosphate (cAMP) in cells expressing
mGlu2 and mGlu3 receptors.

| Compounds of | IC₅₀ (nM) ± SEM | |
|---|---|---|
| U.S. Pat. No. 5,958,960 | mGlu2 | mGlu3 |
| (1S*, 2S*, 4R* 5R*, 6S*) 2- | 3,220 ± 1,310 | 6,270 ± 200 |
| Amino-4-(3-methylureido) | | |
| bicyclo[3.1.0]hexane-2,6- | | |
| dicarboxylic acid | | |
| (1S*, 2S*, 4R*, 5R*, 6S*) | 4,300 ± 1,240 | 11,130 ± 1,080 |
| 4-Acetylamino-2-aminobicyclo | | |
| [3.1.0]hexane-2,6- | | |
| dicarboxylic acid | | |
| 1S*, 2S*, 4R*, 5R*, 6S*) 2- | 4,060 ± 1,400 | 9,520 ± 1,230 |
| Amino-4-benzoylaminobicyclo | | |
| [3.1.0]hexane-2,6- | | |
| dicarboxylic acid | | |
| Compounds of present | EC₅₀ (nM) ± SEM | |
| invention | mGlu2 | mGlu3 |
| (1S*, 2S*, 4S*, 5R*, 6S*) 2- | 426.0 ± 41.82 | 41.11 ± 11.10 |
| Amino-4-(3-methylureido) | | |
| bicyclo[3.1.0]hexane-2,6- | | |
| dicarboxylic acid | | |
| (1S*, 2S*, 4S*, 5R*, 6S*) | 1,350 ± 380 | 1,730 ± 320 |
| 4-Acetylamino-2-aminobicyclo | | |
| [3.1.0]hexane-2,6- | | |
| dicarboxylic acid | | |
| (1S*, 2S*, 4S*, 5R*, 6S*) 2- | 60.68 ± 1.03 | 18.15 ± 2.87 |
| Amino-4-benzoylaminobicyclo | | |
| [3.1.0]hexane-2,6- | | |
| dicarboxylic acid | | |

Each of the compounds of the current invention possess a primary amino group at the C2-position of the bicyclic ring and two carboxylic acid groups, one at the C2-position and one at the C6-position. In general, it has been found that ester and/or amide derivatives of these functional groups are inactive in the receptor binding test. However, it is believed that these compounds are converted in vivo to the corresponding amino diacid and can therefore function as pro-drugs. It will be appreciated that the present invention provides the active amino diacid as well as any pro-drug forms that are capable of generating the active amino diacid in vivo.

The compounds of the present invention are preferably formulated prior to administration. Therefore, another aspect of the present invention is a pharmaceutical formulation comprising a compound of formula I in combination with one or more pharmaceutically-acceptable carriers, diluents, or excipients. The present pharmaceutical formulations are prepared by known procedures using well-known and readily available ingredients. In making the compositions of the present invention, the active ingredient will usually be mixed with a carrier, or diluted by a carrier, or enclosed within a carrier, and may be in the form of a capsule, sachet, paper, or other container. When the carrier serves as a diluent, it may be a solid, semi-solid, or liquid material which acts as a vehicle, excipient, or medium for the active ingredient. The compositions can be in the form of tablets, pills, powders, lozenges, sachets, cachets, elixirs, suspensions, emulsions, solutions, syrups, aerosols, ointments containing, for example, up to 10% by weight of active compound, soft and hard gelatin capsules, suppositories, sterile injectable solutions, dermal patch, subcutaneous implant, and sterile packaged powders.

Some examples of suitable carriers, excipients, and diluents include lactose, dextrose, sucrose, sorbitol, mannitol, starches, gum, acacia, calcium phosphate, alginates, tragacanth, gelatin, calcium silicate, microcrystalline cellulose, polyvinylpyrrolidone, cellulose, water syrup, methyl cellulose, methyl and propyl hydroxybenzoates, talc, magnesium stearate, stearic acid, and mineral oil. The formulations can additionally include lubricating agents, wetting agents (surfactants), emulsifying and suspending agents, preserving agents, sweetening agents, or flavoring agents. Compositions of the invention may be formulated so as to provide quick, sustained, or delayed release of the active ingredient after administration to the patient by employing procedures well known in the art.

The compositions are preferably formulated in a unit dosage form, each dosage containing from about 5 mg to about 500 mg, more preferably about 25 mg to about 300 mg of the active ingredient. As used herein, the term “active ingredient” refers to a compound included within the scope of formula I.

The term “unit dosage form” refers to a physically discrete unit suitable as unitary dosages for human subjects and other mammals, each unit containing a predetermined quantity of active material calculated to produce the desired therapeutic effect, in association with a suitable pharmaceutical carrier, diluent, or excipient.

The following Examples further illustrate the compounds of the present invention and the methods for their synthesis. The Examples are not intended to be limiting to the scope of the invention in any respect, and should not be so construed. All experiments were run under a positive pressure of dry nitrogen or argon. All solvents and reagents were purchased from commercial sources and used as received, unless otherwise indicated. Dry tetrahydrofuran (THF) was obtained by distillation from sodium or sodium benzophenone ketyl prior to use. Proton nuclear magnetic resonance (¹H NMR) spectra were obtained on a GE QE-300 spectrometer at 300.15 MHz, a Bruker AM-500 spectrometer at 500 MHz, or a Bruker AC-200P spectrometer at 200 MHz. Free atom bombardment mass spectroscopy (FABMS) was performed on a VG ZAB-2SE instrument. Field desorption mass spectroscopy (FDMS) was performed using either a VG 70SE or a Varian MAT 731 instrument. Optical rotations were measured with a Perkin-Elmer 241 polarimeter. Chromatographic separation on a Waters Prep 500 LC was generally carried out using a linear gradient of the solvents indicated in the text. The reactions were generally monitored for completion using thin layer chromatography (TLC). Thin layer chromatography was performed using E. Merck Kieselgel 60 F₂₅₄ plates, 5 cm×10 cm, 0.25 mm thickness. Spots were detected using a combination of UV and chemical detection (plates dipped in a ceric ammonium molybdate solution [75 g of ammonium molybdate and 4 g of cerium (IV) sulfate in 500 mL of 10% aqueous sulfuric acid] and then heated on a hot plate). Flash chromatography was performed as described by Still, et al. Still, Kahn, and Mitra, J. Org. Chem., 43, 2923 (1978). Elemental analyses for carbon, hydrogen, and nitrogen were determined on a Control Equipment Corporation 440 Elemental Analyzer, or were performed by the Universidad Complutense Analytical Centre (Facultad de Farmacia, Madrid, Spain). Melting points were determined in open glass capillaries on a Gallenkamp hot air bath melting point apparatus or a Büchi melting point apparatus, and are uncorrected.

The abbreviations, symbols and terms used in the examples have the following meanings.

Ac=acetyl

AllocCl=allyl chloroformate

Anal.=elemental analysis

Bn or Bzl=benzyl

Bu=butyl

BOC=butoxycarbonyl

calcd=calculated

D₂O=deuterium oxide

DCC=dicyclohexylcarbodiimide

DIBAL-H=diisobutyl aluminum hydride

DMAP=dimethylaminopyridine

DMF=dimethylformamide

DMSO=dimethylsulfoxide

EDCI=N-ethyl-N,N′-dimethylaminopropyl carbodiimide

Et=ethyl

EtOAc=ethyl acetate

EtOH=ethanol

FAB=Fast Atom Bombardment (Mass Spectrascopy)

FDMS=field desorption mass spectrum

HOAt=1-hydroxy-7-azabenzotriazole

HOBt=1-hydroxybenzotriazole

HPLC=High Performance Liquid Chromatography

HRMS=high resolution mass spectrum

i-PrOH=isopropanol

IR=Infrared Spectrum

L=liter

Me=methyl

MeOH=methanol

MPLC=Medium Pressure Liquid Chromatography

Mp=melting point

MTBE=t-butyl methyl ether

NaHMDS=sodium hexamethyldisilylamide

NBS=N-bromosuccinimide

NMDBA=1,3-dimethylbarbituric acid

NMR=Nuclear Magnetic Resonance

p-TsCl=para-toluenesulfonyl chloride

p-TsOH=para-toulenesulfonic acid

Ph=phenyl

p.o.=oral administration

i-Pr=isopropyl

Rochelle's Salt=potassium sodium tartrate

SM=starting material

TBS=tert-butyldimethylsilyl

TEA=triethylamine

Temp.=temperature

TFA=trifluoroacetic acid

THF=tetrahydrofuran

TLC=thin layer chromatography

t-ROC=tert-butoxycarbonyl

### Experimental Procedures

### Preparation 1

### (1S*,2S*,4S*,5R*,6S*) Diethyl 2-(N-t-butyloxycarbonylamino)-4-aminobicyclo-[3.1.0]hexane-2,6-dicarboxylic acid

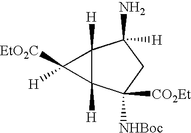

### Method A

### A. (1S*,2S*,4S*,5R*,6R*) Diethyl 2-(N-t-butyloxy-carbonylamino)-4-(p-toluenesulfonyloxy)bicyclo[3.1.0]hexane-2,6-dicarboxylic acid.

p-Toluenesulfonyl chloride (21.3 g, 112 mmol) was added portionwise to a 0° C. solution of (1S*,2S*,4S*,5R*,6R*) diethyl 2-(N-t-butyloxycarbonylamino)-4-hydroxybicyclo-[3.1.0]hexane-2,6-dicarboxylic acid (20.0 g, 56 mmol-synthesis previously described in U.S. Pat. No. 5,958,960 example 8a) in pyridine (80 mL). Upon complete addition the reaction mixture was allowed to warm to room temperature as it stirred over the weekend. The reaction mixture was diluted with EtOAc (2L), washed with cold aqueous NaHSO₄ (3×) then brine, dried over MgSO₄, concentrated in vacuo and purified by SiO₂ chromatography (HPLC: 10% EtOAc/hexanes to 50% EtOAc/hexanes) to yield 26.8 g (52.4 mmol, 94%) of a white foam.

FDMS: M⁺+1=512. Anal. calcd. for C₂₄H₃₃NO₉S: C, 56.35; H, 6.50; N, 2.74. Found: C, 56.13; H, 6.37; N, 2.78.

### B. (1S*,2S*,4R*,5R*,6R*) Diethyl 2-(N-t-butyloxy-carbonylamino)-4-hydroxybicyclo-[3.1.0]hexane-2,6-dicarboxylic acid.

To a room temperature solution of the product from 1a (1.38 g, 2.7.mmol) in dimethyl sulfoxide (25 mL) was added potassium nitrite (2.30 g, 27 mmol) in one portion. The resulting reaction mixture was stirred at 50° C. overnight. The reaction mixture was cooled to room temperature, diluted with EtOAc (500 mL), washed with water (3×) then brine, dried over MgSO₄, concentrated in vauco and purified by SiO₂ chromatography (PC-TLC: 10% EtOAc/hexanes to 20% EtOAc/hexanes) to yield 0.61 g (1.71 mmol, 63%) of a white foam.

FDMS: M⁺+1=358. Anal. calcd. for C₁₇H₂₇NO₇: C, 57.13; H, 7.61; N, 3.92. Found: C, 57.06; H, 7.70; N, 3.81.

### C. (1S*,2S*,4R*,5R*,6R*) Diethyl 2-(N-t-butyloxy-carbonylamino)-4-(p-toluenesulfonyloxy)bicyclo[3.1.0]hexane-2,6-dicarboxylic acid.

p-Toluenesulfonyl chloride (3.8 g, 20 mmol) was added in one portion to a 0° C. solution of the product from 1b (3.6 g, 10 mmol) in pyridine (15 mL). Upon complete addition the reaction mixture was allowed to warm to room temperature as it stirred overnight. The reaction mixture was diluted with EtOAc (500 mL), washed with cold aqueous NaHSO₄ (3×) then brine, dried over MgSO₄, concentrated in vacuo and purified by SiO₂ chromatography (HPLC: 10% EtOAc/hexanes to 50% EtOAc/hexanes) to yield 4.71 g (9.2 mmol, 92%) of a white foam.

FDMS: M⁺+1=512. Anal. calcd. for C₂₄H₃₃NO₉S: C, 56.35; H, 6.50; N, 2.74. Found: C, 55.99; H, 6.15; N, 2.78.

### D. (1S*,2S*,4S*,5R*,6S*) Diethyl 2-(N-t-butyloxy-carbonylamino)-4-azidobicyclo-[3.1.0]hexane-2,6-dicarboxylic acid.

To a room temperature solution of the product from 1c (1.0 g, 2.0 mmol) in dimethyl sulfoxide (10 mL) was added sodium azide (0.39 g, 6.0 mmol) in one portion. The resulting reaction mixture was stirred at 45° C. overnight. The reaction mixture was cooled to room temperature, diluted with EtOAc (500 mL), washed with water (3×) then brine, dried over MgSO₄, concentrated in vauco and purified by SiO₂ chromatography (PC-TLC: 10% EtOAc/hexanes to 20% EtOAc/hexanes) to yield 0.76 g (2.0 mmol, 86%) of a white foam.

FDMS: M⁺+1=383. Anal. calcd. for C₁₇H₂₆N₄O₆: C, 53.39; H, 6.85; N, 14.65. Found: C, 53.39; H, 6.93; N, 14.36.

### E. (1S*,2S*,4S*,5R*,6S*) Diethyl 2-(N-t-butyloxy-carbonylamino)-4-aminobicyclo-[3.1.0]hexane-2,6-dicarboxylic acid.

To a room temperature solution of the product from 1d (0.65 g, 1.7 mmol) in anhydrous tetrahydrofuran (15 mL) was added triphenylphosphine (0.53 g, 2.0 mmol) in one portion. The resulting reaction mixture was stirred at room temperature overnight. Water (2 mL) was added to the reaction mixture and subsequently stirred overnight at room temperature. The reaction mixture was partitioned between saturated aqueous sodium bicarbonate and EtOAc. The product was extracted with EtOAc. All organics were combined, washed with water then brine, dried over K₂CO₃, concentrated in vauco and purified by SiO₂ chromatography (PC-TLC: 10% EtOAc/hexanes to 90% EtOAc/hexanes) to yield 0.56 g (1.57 mmol, 92%) of a white foam.

FDMS: M⁺+1=357. Anal. calcd. for C₁₇H₂₈N₂O₆: C, 57.29; H, 7.92; N, 7.86. Found: C, 57.00; H, 7.70; N, 7.65.

### Method B

### A. (1S*,2S*,4R*,5R*,6R*) Diethyl 2-(N-t-butyloxy-carbonylamino)-4-bromobicyclo-[3.1.0]hexane-2,6-dicarboxylic acid.

Bromine was added dropwise to a room temperature solution of triphenylphosphine (0.40 g, 1.5 mmol) in CH₂Cl₂ (25 mL) until a light yellow color persisted. Additional triphenylphosphine was added to the reaction mixture until the solution became colorless. The reaction mixture was chilled to 0° C. and a solution of (1S*,2S*,4S*,5R*,6R*) diethyl 2-(N-t-butyloxycarbonylamino)-4-hydroxy-bicyclo[3.1.0]hexane-2,6-dicarboxylic acid (0.36 g, 1.0 mmol) in a 1:1 mixture of CH₂Cl₂/pyridine (20 mL total volume) was added dropwise to the reaction mixture. Upon complete addition the reaction mixture was allowed to warm to room temperature as it stirred overnight. The reaction mixture was diluted with-Et₂O (500 mL), washed with cold aqueous NaHSO₄ (3×) then brine, dried over MgSO₄, concentrated in vacuo and purified by SiO₂ chromatography (PC-TLC: 10% EtOAc/hexanes to 50% EtOAc/hexanes) to yield 0.41 g (0.98 mmol, 97%) of a white foam.

FDMS: M⁺+18(NH₄⁺)=439. Anal. calcd. for C₁₇H₂₆BrNO₆: C, 48.58; H, 6.24; N, 3.33. Found: C, 48.31; H, 6.04; N, 3.15.

### B. (1S*,2S*,4S*,5R*,6S*) Diethyl 2-(N-t-butyloxy-carbonylamino)-4-azidobicyclo[3.1.0] hexane-2,6-dicarboxylic acid.

To a room temperature solution of the product from if (13.2 g, 31.4 mmol) in dimethyl sulfoxide (150 mL) was added sodium azide (4.1 g, 62.0 mmol) in one portion. The resulting reaction mixture was stirred at 50° C. overnight. The reaction mixture was cooled to room temperature, diluted with EtOAc (2 L), washed with water (3×) then brine, dried over MgSO₄, concentrated in vauco and purified by SiO₂ chromatography (HPLC: 10% EtOAc/hexanes to 50% EtOAc/hexanes) to yield 11.3 g (29.5 mmol, 94%) of a white foam.

FDMS: M⁺+1=383. Anal. calcd. for C₁₇H₂₆N₄O₆: C, 53.39; H, 6.85; N, 14.65. Found: C, 53.28; H, 6.79; N, 14.41.

### C. (1S*,2S*,4S*,5R*,6S*) Diethyl 2-(N-t-butyloxy-carbonylamino)-4-aminobicyclo-[3.1.0]hexane-2,6-dicarboxylic acid.

To a room temperature solution of the product from id (0.65 g, 1.7 mmol) in anhydrous tetrahydrofuran (15 mL) was added triphenylphosphine (0.53 g, 2.0 mmol) in one portion. The resulting reaction mixture was stirred at room temperature overnight. Water (2 mL) was added to the reaction mixture and subsequently stirred overnight at room temperature. The reaction mixture was partitioned between saturated aqueous sodium bicarbonate and EtOAc. The product was extracted with EtOAc. All organics were combined, washed with water then brine, dried over K₂CO₃, concentrated in vacuo and purified by SiO₂ chromatography (PC-TLC: 10% EtOAc/hexanes to 90% EtOAc/hexanes) to yield 0.56 g (1.57 mmol, 92%) of a white foam.

FDMS: M⁺+1=357. Anal. calcd. for C₁₇H₂₈N₂O₆: C, 57.29; H, 7.92; N, 7.86. Found: C, 57.00; H, 7.70; N, 7.65.

### EXAMPLE 1

### Synthesis of (1S*,2S*,4S*,5R*,6S*) 2-amino-4-[3-(2-methoxyphenyl)ureido]bicyclo[3.1.0]hexane-2,6-dicarboxylic acid

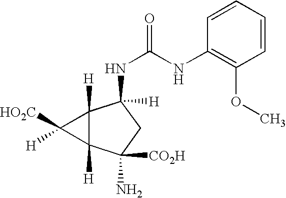

### A. (1S*,2S*,4S*,5R*,6S*) Diethyl 2-(N-t-butyloxy-carbonylamino)-4-[3-(2-methoxy-phenyl)ureido]bicyclo[3.1.0]-hexane-2,6-dicarboxylic acid.

A room temperature solution of the product of Preparation 1 (0.25 g, 0.70 mmol) in CH₂Cl₂ (10 mL) was treated in one portion with 2-methoxyphenyl isocyanate (0.13 g, 0.85 mmol). The resulting reaction mixture was allowed to stir at room temperature overnight. The reaction mixture was concentrated in vacuo and purified by SiO₂ chromatography (PC-TLC: 10% EtOAc/hexanes to 90% EtOAc/hexanes) to yield 0.34 g (0.67 mmol, 96%) of a white foam.

FDMS: M⁺+1=506. Anal. calcd. for C₂₅H₃₅N₃O₈.0.1 eq. EtOAc: C, 59.31; H, 7.02; N, 8.17. Found: C, 58.97; H, 6.94; N, 8.06.

### B. (1S*,2S*,-4S*,5R*,6S*) 2-amino-4-[3-(2-methoxyphenyl)-ureido]bicyclo-[3.1.0]hexane-2,6-dicarboxylic acid.

A 0° C. solution of the product of example 1a ( 0.31 g, 0.61 mmol) in EtOAc (35mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (10 mL total volume) and stirred at room temperature overnight. The reaction mixture was adjusted to pH=7 with 1N HCl and concentrated to dryness. The solids were reconstituted in water, adjusted to pH=12 with 1N NaOH and purified by anion exchange chromatography (Bio-Rad® AG1-X8 resin; acetate form converted to hydroxide form). The product was eluted from the column with 3N AcOH and concentrated to dryness in vacuo to afford crude product. Trituration in 2-propanol:water (5:1) followed by drying under vacuum at 80° C. afforded 0.20 g (0.57 mmol, 94%) of the desired product as a white solid.

mp=>250° C. FDMS: M⁺+1=350. Anal. calcd. for C₁₆H₁₉N₃O₆.1.0 eq. H₂O: C, 52.31; H, 5.76; N, 11.44. Found: C, 52.58; H, 5.52; N, 11.49.

### EXAMPLE 2

### Synthesis of (1S*,2S*,4S*,5R*,6S*) 2-amino-4-[3-(3-methoxyphenyl)ureido]bicyclo[3.1.0]hexane-2,6-dicarboxylic acid

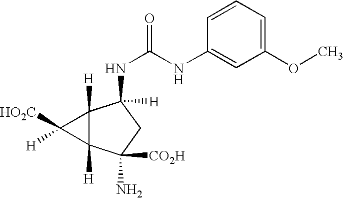

### A. (1S*,2S*,4S*,5R*,6S*) Diethyl 2-(N-t-butyloxycarbonyl-amino)-4-[3-(3-methoxy-phenyl)ureido]bicyclo[3.1.0]hexane-2,6-dicarboxylic acid.

A room temperature solution of the product of Preparation 1 (0.25 g, 0.70 mmol) in CH₂Cl₂ (10 mL) was treated in one portion with 3-methoxyphenyl isocyanate (0.13 g, 0.85 mmol). The resulting reaction mixture was allowed to stir at room temperature overnight. The reaction mixture was concentrated in vacuo and purified by SiO₂ chromatography (PC-TLC: 10% EtOAc/hexanes to 90% EtOAc/hexanes) to yield 0.32 g (0.63 mmol, 90%) of a white foam.

FDMS: M⁺+1=506. Anal. calcd. for C₂₅H₃₅N₃O₈.0.1 eq. CH₂Cl₂: C, 58.64; H, 6.90; N, 8.17. Found: C, 58.36; H, 6.74; N, 8.06.

### B. (1S*,2S*,4S*,5R*,6S*) 2-amino-4-[3-(3-methoxyphenyl)-ureido]bicyclo-[3.1.0]hexane-2,6-dicarboxylic acid.

A 0° C. solution of the product of Part A ( 0.30 g, 0.60 mmol) in EtOAc (35mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (10 mL total volume) and stirred at room temperature overnight. The reaction mixture was adjusted to pH=7 with 1N HCl and concentrated to dryness. The solids were reconstituted in water, adjusted to pH=12 with 1N NaOH and purified by anion exchange chromatography (Bio-Rad® AG1-X8 resin; acetate form converted to hydroxide form). The product was eluted from the column with 3N AcOH and concentrated to dryness in vacuo to afford crude product. Trituration in 2-propanol:water (5:1) followed by drying under vacuum at 80° C. afforded 0.16 g (0.46 mmol, 76%) of the desired product as a white solid.

mp=>250° C. FDMS: M⁺+1=350. Anal. calcd. for C₁₆H₁₉N₃O₆.0.3 eq. H₂O.0.4 eq. 2-propanol: C, 54.54; H, 6.07; N, 11.09. Found: C, 54.39; H, 5.84; N, 10.82.

### EXAMPLE 3

### Synthesis of (1S*,2S*,4S*,5R*,6S*) 2-amino-4-[3-(4-methoxyphenyl)ureido]bicyclo[3.1.0]hexane-2,6-dicarboxylic acid

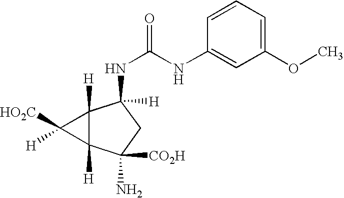

### A. (1S*,2S*,4S*,5R*,6S*) Diethyl 2-(N-t-butyloxycarbonyl-amino)-4-[3-(4-methoxy-phenyl)ureido]bicyclo[3.1.0]hexane-2,6-dicarboxylic acid.

A room temperature solution of the product of preparation 1 (0.25 g, 0.70 mmol) in CH₂Cl₂ (10 mL) was treated in one portion with 4-methoxyphenyl isocyanate (0.13 g, 0.85 mmol). The resulting reaction mixture was allowed to stir at room temperature overnight. The reaction mixture was concentrated in vacuo and purified by SiO₂ chromatography (PC-TLC: 10% EtOAc/hexanes to 90% EtOAc/hexanes) to yield 0.30 g (0.60 mmol, 85%) of a white foam.

FDMS: M⁺+1=506. Anal. calcd. for C₂₅H₃₅N₃O₈: C, 59.39; H, 6.98; N, 8.31. Found: C, 58.36; H, 6.74; N, 8.06.

### B. (1S*,2S*,4S*,5R*,6S*) 2-amino-4-[3-(4-methoxyphenyl)-ureido]bicyclo[3.1.0]-hexane-2,6-dicarboxylic acid.

A 0° C. solution of the product of example 3a ( 0.30 g, 0.60 mmol) in EtOAc (35mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (15 mL total volume) and stirred at room temperature overnight. The reaction mixture was adjusted to pH=7 with 1N HCl and concentrated to dryness. The solids were reconstituted in water, adjusted to pH=12 with 1N NaOH and purified by anion exchange chromatography (Bio-Rad® AG1-X8 resin; acetate form converted to hydroxide form). The product was eluted from the column with 3N AcOH and concentrated to dryness in vacuo to afford crude product. Trituration in acetone:water (5:1) followed by drying under vacuum at 80° C. afforded 0.12 g (0.34 mmol, 62%) of the desired product as a white solid.

mp=>250° C. FDMS: M⁺+1=350. Anal. calcd. for C₁₆H₁₉N₃O₆.0.4 eq. H₂O: C, 53.90; H, 5.60; N,. 11.79. Found: C, 53.76; H, 5.14; N, 11.43.

### EXAMPLE 4

### Synthesis of (1S*,2S*,4S*,5R*,6S*) 2-amino-4-[3-(2-methylphenyl)ureido]bicyclo[3.1.0]hexane-2,6-dicarboxylic acid

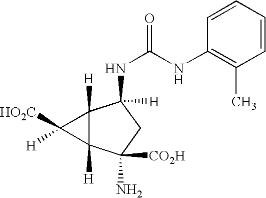

### A. (1S*,2S*,4S*,5R*,6S*) Diethyl 2-(N-t-butyloxycarbonyl-amino)-4-[3-(2-methyl-phenyl)ureido]bicyclo[3.1.0]hexane-2,6-dicarboxylic acid.

A room temperature solution of the product of preparation 1 (0.25 g, 0.70 mmol) in CH₂Cl₂ (10 mL) was treated in one portion with 2-methylphenyl isocyanate (0.12 g, 0.85 mmol). The resulting reaction mixture was allowed to stir at room temperature overnight. The reaction mixture was concentrated in vacuo and purified by SiO₂ chromatography (PC-TLC: 10% EtOAc/hexanes to 90% EtOAc/hexanes) to yield 0.32 g (0.65 mmol, 93%) of a white foam.

FDMS: M⁺+1=490. Anal. calcd. for C₂₅H₃₅N₃O₇.0.1 eq. CH₂Cl₂: C, 60.53; H, 7.12; N, 8.44. Found: C, 60.61; H, 7.01; N, 8.27.

### B. (1S*,2S*,4S*,5R*,6S*) 2-amino-4-[3-(2-methylphenyl)-ureido]bicyclo[3.1.0]hexane-2,6-dicarboxylic acid.

A 0° C. solution of the product of example 4a ( 0.30 g, 0.60 mmol) in EtOAc (35mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (15 mL total volume) and stirred at room temperature overnight. The reaction mixture was adjusted to pH=7 with 1N HCl and concentrated to dryness. The solids were reconstituted in water, adjusted to pH=12 with 1N NaOH and purified by anion exchange chromatography (Bio-Rad® AG1-X8 resin; acetate form converted to hydroxide form). The product was eluted from the column with 3N AcOH and concentrated to dryness in vacuo to afford crude product. Trituration in 2-propanol:water (5:1) followed by drying under vacuum at 80° C. afforded 0.12 g (0.34 mmol, 62%) of the desired product as a white solid.

mp=>250° C. FDMS: M⁺+1=334. Anal. calcd. for C₁₆H₁₉N₃O₅.1.1 eq. H₂O .0.3 eq. AcOH: C, 53.75; H, 6.07; N, 11.19. Found: C, 53.40; H, 5.70; N, 11.03.

### EXAMPLE 5

### Synthesis of (1S*,2S*,4S*,5R*,6S*) 2-amino-4-[3-(3-methylphenyl)ureido]bicyclo[3.1.0]hexane-2,6-dicarboxylic acid

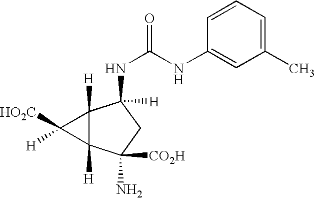

### A. (1S*,2S*,4S*,5R*,6S*) Diethyl 2-(N-t-butyloxycarbonyl-amino)-4-[3-(3-methyl-phenyl)ureido]bicyclo[3.1.0]hexane-2,6-dicarboxylic acid.

A room temperature solution of the product of preparation 1 (0.25 g, 0.70 mmol) in CH₂Cl₂ (10 mL) was treated in one portion with 3-methylphenyl isocyanate (0.12 g, 0.85 mmol). The resulting reaction mixture was allowed to stir at room temperature overnight. The reaction mixture was concentrated in vacuo and purified by SiO₂ chromatography (PC-TLC: 10% EtOAc/hexanes to 90% EtOAc/hexanes) to yield 0.31 g (0.63 mmol, 90%) of a white foam.

FDMS: M⁺+1=490. Anal. calcd. for C₂₅H₃₅N₃O₇.0.1 eq. CH₂Cl₂: C, .60.53; H, 7.12; N, 8.44. Found: C, 60.74; H, 7.14; N, 8.37.

### B. (1S*,2S*,4S*,5R*,6S*) 2-amino-4-[3-(3-methylphenyl)-ureido]bicyclo[3.1.0]hexane-2,6-dicarboxylic acid.

A 0° C. solution of the product of example 5a ( 0.30 g, 0.60 mmol) in EtOAc (35mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (15 mL total volume) and stirred at room temperature overnight. The reaction mixture was adjusted to pH=7 with 1N HCl and concentrated to dryness. The solids were reconstituted in water, adjusted to pH=12 with 1N NaOH and purified by anion exchange chromatography (Bio-Rad® AG1-X8 resin; acetate form converted to hydroxide form). The product was eluted from the column with 3N AcOH and concentrated to dryness in vacuo to afford crude product. Trituration in 2-propanol:water (5:1) followed by drying under vacuum at 80° C. afforded 0.12 g (0.34 mmol, 62%) of the desired product as a white solid.

mp=>250° C. FDMS: M⁺+1=334. Anal. calcd. for C₁₆H₁₉N₃O₅.0.6 eq. H₂O: C, 55.84; H, 5.92; N, 12.21. Found: C, 55.75; H, 5.84; N, 12.13.

### EXAMPLE 6

### Synthesis of (1S*,2S*,4S*,5R*,6S*) 2-amino-4-[3-(4-methylphenyl)ureido]bicyclo[3.1.0]hexane-2,6-dicarboxylic acid

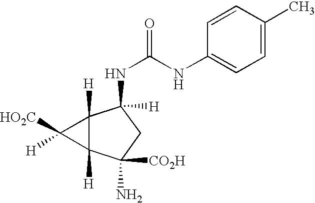

### A. (1S*,2S*,4S*,5R*,6S*) Diethyl 2-(N-t-butyloxycarbonyl-amino)-4-[3-(4-methyl-phenyl)ureido]bicyclo[3.1.0]hexane-2,6-dicarboxylic acid.

A room temperature solution of the product of preparation 1 (0.25 g, 0.70 mmol) in CH₂Cl₂ (10 mL) was treated in one portion with 4-methylphenyl isocyanate (0.12 g, 0.85 mmol). The resulting reaction mixture was allowed to stir at room temperature overnight. The reaction mixture was concentrated in vacuo and purified by SiO₂ chromatography (PC-TLC: 10% EtOAc/hexanes to 20% EtOAc/hexanes) to yield 0.34 g (0.69 mmol, 99%) of a white foam.

FDMS: M⁺+1=490. Anal. calcd. for C₂₅H₃₅N₃O₇.0.1 eq. CH₂Cl₂: C, 60.53; H, 7.12; N, 8.44. Found: C, 60.71; H, 7.20; N, 8.44.

### B. (1S*,2S*,4S*,5R*,6S*) 2-amino-4-[3-(3-methylphenyl)-ureido]bicyclo[3.1.0]hexane-2,6-dicarboxylic acid.

A 0° C. solution of the product of example 6a ( 0.30 g, 0.60 mmol) in EtOAc (35mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (15 mL total volume) and stirred at room temperature overnight. The reaction mixture was adjusted to pH=7 with 1N HCl and concentrated to dryness. The solids were reconstituted in water, adjusted to pH=12 with 1N NaOH and purified by anion exchange chromatography (Bio-Rad® AG1-X8 resin; acetate form converted to hydroxide form). The product was eluted from the column with 3N AcOH and concentrated to dryness in vacuo to afford crude product. Trituration in 2-propanol:water (5:1) followed by drying under vacuum at 80° C. afforded 0.12 g (0.36 mmol, 59%) of the desired product as a white solid.

mp=charred>220° C. FDMS: M⁺+1=334. Anal. calcd. for C₁₆H₁₉N₃O₅: C, 57.65; H, 5.74; N, 12.60. Found: C, 60.55; H, 6.52; N, 12.26.

### EXAMPLE 7

### Synthesis of (1S*,2S*,4S*,5R*,6S*) 2-amino-4-[3-(2-chlorophenyl)ureido]bicyclo[3.1.0]hexane-2,6-dicarboxylic acid

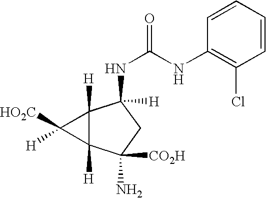

### A. (1S*,2A*,4S*,5S*,6S*) Diethyl 2-(N-t-butyloxycarbonyl-amino)-4-[3-(2-chloro-phenyl)ureido]bicyclo[3.1.0]hexane-2,6-dicarboxylic acid.

A room temperature solution of the product of preparation 1 (0.25 g, 0.70 mmol) in CH₂Cl₂ (10 mL) was treated in one portion with 2-chlorophenyl isocyanate (0.15 g, 1.0 mmol). The resulting reaction mixture was allowed to stir at room temperature overnight. The reaction mixture was concentrated in vacuo and purified by SiO₂ chromatography (PC-TLC: 10% EtOAc/hexanes to 50% EtOAc/hexanes) to yield 0.35 g (0.69 mmol, 98%) of a white foam.

FDMS: M⁺+1=510. Anal. calcd. for C₂₄H₃₂ClN₃O₇.0.1 eq. CH₂C1₂: C, 55.83; H, 6.26; N, 8.10. Found: C, 55.95; H, 6.21; N, 8.14.

### B. (1S*,2S*,4S*,5R*,6S*) 2-amino-4-[3-(2-chlorophenyl)-ureido]bicyclo-[3.1.0]hexane-2,6-dicarboxylic acid.

A 0° C. solution of the product of example 7a (0.30 g, 0.59 mmol) in EtOAc (35mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (15 mL total volume) and stirred at room temperature overnight. The reaction mixture was adjusted to pH=7 with 1N HCl and concentrated to dryness. The solids were reconstituted in water, adjusted to pH=12 with 1N NaOH and purified by anion exchange chromatography (Bio-Rad® AG1-X8 resin; acetate form converted to hydroxide form). The product was eluted from the column with 3N AcOH and concentrated to dryness in vacuo to afford crude product. Trituration in 2-propanol:water (5:1) followed by drying under vacuum at 80° C. afforded 0.04 g (0.11 mmol, 19%) of the desired product as a white solid.

mp=>250° C. FDMS: M−1=352. Anal. calcd. for C₁₅H₁₆ClN₃O₅.1.1 eq. H₂O: C, 48.23; H, 4.91; N, 11.25. Found: C, 48.00; H, 4.39; N, 11.09.

### EXAMPLE 8

### Synthesis of (1S*,2S*,4S*,5R*,6S*) 2-amino-4-[3-(3-chlorophenyl)ureido]bicyclo[3.1.0]hexane-2,6-dicarboxylic acid

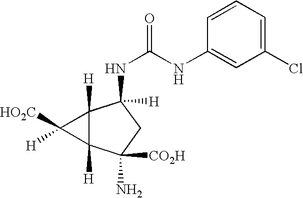

### A. (1S*,2S*,4S*,5R*,6S*) Diethyl 2-(N-t-butyloxycarbonyl-amino)-4-[3-(3-chloro-phenyl)ureido]bicyclo[3.1.0]hexane-2,6-dicarboxylic acid.

A room temperature solution of the product of preparation 1 (0.25 g, 0.70 mmol) in CH₂Cl₂ (10 mL) was treated in one portion with 3-chlorophenyl isocyanate (0.15 g, 1.0 mmol). The resulting reaction mixture was allowed to stir at room temperature overnight. The reaction mixture was concentrated in vacuo and purified by SiO₂ chromatography (PC-TLC: 10% EtOAc/hexanes to 50% EtOAc/hexanes) to yield 0.30 g (0.59 mmol, 84%) of a white foam.

FDMS: M⁺+1=510. Anal. calcd. for C₂₄H₃₂ClN₃O₇.0.1 eq. CH₂Cl₂: C, 55.83; H, 6.26; N, 8.10. Found: C, 55.63; H, 6.31; N, 8.02.

### B. (1S*,2S*,4S*,5R*,6S*) 2-amino-4-[3-(3chlorophenyl)-ureido]bicyclo[3.1.0]hexane-2,6-dicarboxylic acid.

A 0° C. solution of the product of example 8a (0.25 g, 0.49 mmol) in EtOAc (35mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (15 mL total volume) and stirred at room temperature overnight. The reaction mixture was adjusted to pH=7 with 1N HCl and concentrated to dryness. The solids were reconstituted in water, adjusted to pH=12 with 1N NaOH and purified by anion exchange chromatography (Sio-Rad® AG1-X8 resin; acetate form converted to hydroxide form). The product was eluted from the column with 3N AcOH and concentrated to dryness in vacuo to afford crude product. Trituration in 2-propanol:water (5:1) followed by drying under vacuum at 80° C. afforded 0.06 g (0.17 mmol, 35%) of the desired product as a white solid.

mp=>250° C. FDMS: M−1=352. Anal. calcd. for C₁₅H₁₆ClN₃O₅.0.7 eq. H₂O: C, 49.17; H, 4.79; N, 11.47. Found: C, 48.96; H, 4.54; N, 11.23.

### EXAMPLE 9

### Synthesis of (1S*,2S*,4S*,5R*,6S*) 2-amino-4-[3-(4-chlorophenyl)ureido]bicyclo[3.1.0)hexane-2,6-dicarboxylic acid

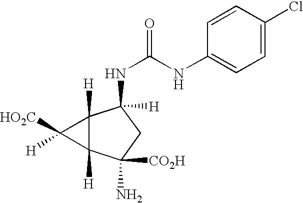

### A. (1S*,2S*,4S*,5R*,6S*) Diethyl 2-(N-t-butyloxycarbonyl-amino)-4-[3-(4-chloro-phenyl)ureido]bicyclo[3.1.0]hexane-2,6-dicarboxylic acid.

A room temperature solution of the product of preparation 1 (0.25 g, 0.70 mmol) in CH₂Cl₂ (10 mL) was treated in one portion with 4-chlorophenyl isocyanate (0.15 g, 1.0 mmol). The resulting reaction mixture was allowed to stir at room temperature overnight. The reaction mixture was concentrated in vacuo and purified by SiO₂ chromatography (PC-TLC: 10% EtOAc/hexanes to 50% EtOAc/hexanes) to yield 0.29 g (0.57 mmol, 81%) of a white foam.

FDMS: M⁺+1=510. Anal. calcd. for C₂₄H₃₂ClN₃O₇.0.2 eq. CH₂Cl₂: C, 55.16; H, 6.20; N. 7.97. Found: C, 55.44; H, 6.19; N. 7.96.

### B. (1S*,2S*,4S*,5R*,6S*) 2-amino-4-[3-(4-chlorophenyl)-ureido]bicyclo[3.1.0]hexane-2,6-dicarboxylic acid.

A 0° C. solution of the product of example 9a ( 0.25 g, 0.49 mmol) in EtOAc (35mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (15 mL total volume) and stirred at room temperature overnight. The reaction mixture was adjusted to pH=7 with 1N HCl and concentrated to dryness. The solids were reconstituted in water, adjusted to pH=12 with 1N NaOH and purified by anion exchange chromatography (Bio-Rad® AG1-X8 resin; acetate form converted to hydroxide form). The product was eluted from the column with 3N AcOH and concentrated to dryness in vacuo to afford crude product. Trituration in 2-propanol:water (5:1) followed by drying under vacuum at 80° C. afforded 0.05 g (0.15 mmol, 31%) of the desired product as a white solid.

mp=>250° C. FDMS: M−1=352. Anal. calcd. for C₁₅H₁₆ClN₃O₅.1.6 eq. H₂O: C, 47.09; H, 5.06; N, 10.98. Found: C, 46.86; H, 4.65; N, 10.72.

### EXAMPLE 10

### Synthesis of (1S*,2S*,4S*,5R*,6S*) 2-amino-4-[3-(2-fluorophenyl)ureido]-bicyclo[3.1.0]hexane-2,6-dicarboxylic acid

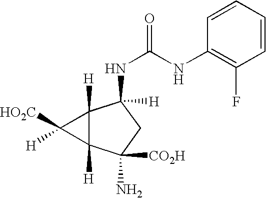

### A. (1S*,2S*,4S*,5R*,6S*) Diethyl 2-(N-t-butyloxycarbonyl-amino)-4-[3-(2-fluoro-phenyl)ureido]bicyclo[3.1.0]hexane-2,6-dicarboxylic acid.

A room temperature solution of the product of preparation 1 (0.25 g, 0.70 mmol) in CH₂Cl₂ (10 mL) was treated in one portion with 2-fluorophenyl isocyanate (0.12 g, 0.85 mmol). The resulting reaction mixture was allowed to stir at room temperature overnight. The reaction mixture was concentrated in vacuo and purified by SiO₂ chromatography (PC-TLC: 10% EtOAc/hexanes to 50% EtOAc/hexanes) to yield 0.32 g (0.64 mmol, 93%) of a white foam.

FDMS: M⁺+1=494. Anal. calcd. for C₂₄H₃₂FN₃O₇.0.2 eq. CH₂Cl₂: C, 56.93; H, 6.40; N, 8.23. Found: C, 57.12; H, 6.16; N, 8.24.

### B. (1S*,2S*,4S*,5R*,6S*) 2-amino-4-[3-(2-fluorophenyl)-ureido]bicyclo[3.1.0]hexane-2,6-dicarboxylic acid.

A 0° C. solution of the product of example 10a (0.25 g, 0.51 mmol) in EtOAc (35mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (15 mL total volume) and stirred at room temperature overnight. The reaction mixture was adjusted to pH=7 with 1N HCl and concentrated to dryness. The solids were reconstituted in water, adjusted to pH=12 with 1N NaOH and purified by anion exchange chromatography (Bio-Rad® AG1-X8 resin; acetate form converted to hydroxide form). The product was eluted from the column with 3N AcOH and concentrated to dryness in vacuo to afford crude product. Trituration in 2-propanol:water (5:1) followed by drying under vacuum at 80° C. afforded 0.10 g (0.30 mmol, 59%) of the desired product as a white solid.

mp=>270° C. FDMS: M⁺+1=338. Anal. calcd. for C₁₅H₁₆FN₃O₅.1.8 eq. H₂O: C, 48.73; H, 5.34; N, 11.37. Found: C, 48.56; H, 5.00; N, 11.59.

### EXAMPLE 11

### Synthesis of (1S*,2S*,4S*,5R*,6S*) 2-Amino-4-[3-(3-fluorophenyl)ureido]bicyclo[3.1.0]hexane-2,6-dicarboxylic acid

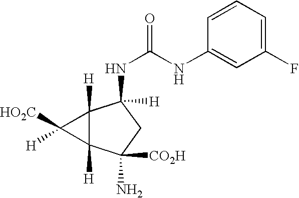

### A. (1S*,2S*,4S*,5R*,6S*) Diethyl 2-(N-t-butyloxycarbonyl-amino)-4-[3-(3-fluoro-phenyl)ureido]bicyclo[3.1.0]hexane-2,6-dicarboxylic acid.

A room temperature solution of the product of preparation 1 (0.25 g, 0.70 mmol) in CH₂Cl₂ (10 mL) was treated in one portion with 3-fluorophenyl isocyanate (0.12 g, 0.85 mmol). The resulting reaction mixture was allowed to stir at room temperature overnight. The reaction mixture was concentrated in vacuo and purified by SiO₂ chromatography (PC-TLC: 10% EtOAc/hexanes to 50% EtOAc/hexanes) to yield 0.34 g (0.69 mmol, 98%) of a white foam.

FDMS: M⁺+1=494. Anal. calcd. for C₂₄H₃₂FN₃O₇.0.1 eq. CH₂Cl₂: C, 57.66; H, 6.47; N, 8.37. Found: C, 57.40; H, 6.33; N, 8.18.

### B. (1S*,2S*,4S*,5R*,6S*) 2-amino-4-[3-(3-fluorophenyl)-ureido]bicyclo[3.1.0]hexane-2,6-dicarboxylic acid.

A 0° C. solution of the product of example 11a (0.30 g, 0.61 mmol) in EtOAc (35mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (15 mL total volume) and stirred at room temperature overnight. The reaction mixture was adjusted to pH=7 with 1N HCl and concentrated to dryness. The solids were reconstituted in water, adjusted to pH=12 with 1N NaOH and purified by anion exchange chromatography (Bio-Rad® AG1-X8 resin; acetate form converted to hydroxide form). The product was eluted from the column with 3N AcOH and concentrated to dryness in vacuo to afford crude product. Trituration in 2-propanol:water (5:1) followed by drying under vacuum at 80° C. afforded 0.17 g (0.50 mmol, 83%) of the desired product as a white solid.

mp=>250° C. FDMS: M⁺+1=338. Anal. calcd. for C₁₅H₁₆FN₃O₅.1.5 eq. H₂O: C, 49.45; H, 5.26; N, 11.53. Found: C, 49.22; H, 5.18; N, 11.65.

### EXAMPLE 12

Synthesis of (1S*,2S*,4S*,5R*,6S*) 2-amino-4-[3-(4-fluorophenyl)ureido]bicyclo[3.1.0]hexane-2,6-dicarboxylic acid

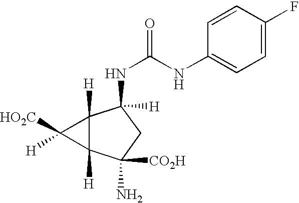

### A. (1S*,2S*,4S*,5R*,6S*) Diethyl 2-(N-t-butyloxycarbonyl-amino)-4-[3-(4-fluoro-phenyl)ureido]bicyclo[3.1.0]hexane-2,6-dicarboxylic acid

A room temperature solution of the product of preparation 1 (0.25 g, 0.70 mmol) in CH₂Cl₂ (10 mL) was treated in one portion with 4-fluorophenyl isocyanate (0.12 g, 0.85 mmol). The resulting reaction mixture was allowed to stir at room temperature overnight. The reaction mixture was concentrated in vacuo and purified by SiO₂ chromatography (PC-TLC: 10% EtOAc/hexanes to 50% EtOAc/hexanes) to yield 0.35 g (0.70 mmol, 100%) of a white foam.

FDMS: M⁺+1=494. Anal. calcd. for C₂₄H₃₂FN₃O₇.0.1 eq. CH₂Cl₂: C, 57.66; H, 6.47; N, 8.37. Found: C, 57.55; H, 6.40; N, 8.24.

### B. (1S*,2S*,4S*,5R*,6S*) 2-amino-4-[3-(4-fluorophenyl)-ureido]bicyclo[3.1.0]hexane-2,6-dicarboxylic acid

A 0° C. solution of the product of example 12a (0.30 g, 0.61 mmol) in EtOAc (35 mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (15 mL total volume) and stirred at room temperature overnight. The reaction mixture was adjusted to pH=7 with 1N HCl and concentrated to dryness. The solids were reconstituted in water, adjusted to pH=12 with 1N NaOH and purified by anion exchange chromatography (Bio-Rad® AG1-X8 resin; acetate form converted to hydroxide form). The product was eluted from the column with 3N AcOH and concentrated to dryness in vacuo to afford crude product. Trituration in 2-propanol:water (5:1) followed by drying under vacuum at 80° C. afforded 0.17 g (0.50 mmol, 83%) of the desired product as a white solid.

mp=charred>220° C. FDMS: M⁺+1=338. Anal. calcd. for C₁₅H₁₆FN₃O₅.1.5 eq. H₂O: C, 49.45; H, 5.26; N, 11.53. Found: C, 49.08; H, 5.23; N, 11.61.

### EXAMPLE 13

### Synthesis of (1S*,2S*,4S*,5R*,6S*) 2-amino-4-[3-(2,6-dichlorophenyl)ureido]bicyclo[3.1.0]hexane-2,6-dicarboxylic acid

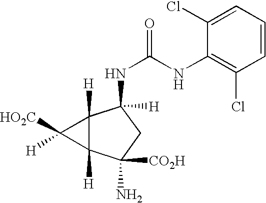

### A. (1S*,2S*,4S*,5R*,6S*) Diethyl 2-(N-t-butyloxycarbonyl-amino)-4-[3-(2,6-dichloro-phenyl)ureido]-bicyclo[3.1.0]hexane-2,6-dicarboxylic acid

A room temperature solution of the product of preparation 1 (0.25 g, 0.70 mmol) in CH₂Cl₂ (10 mL) was treated in one portion with 2,6-dichlorophenyl isocyanate (0.16 g, 0.85 mmol). The resulting reaction mixture was allowed to stir at room temperature overnight. The reaction mixture was concentrated in vacuo and purified by SiO₂ chromatography (PC-TLC: 10% EtOAc/hexanes to 50% EtOAc/hexanes) to yield 0.35 g (0.64 mmol, 92%) of a white foam.

FDMS: M−1=542. Anal. calcd. for C₂₄H₃₁Cl₂N₃O₇.0.1 eq. CH₂Cl₂: C, 52.35; H, 5.69; N, 7.60. Found: C, 52.34; H, 5.55; N, 7.46.

### B. (1S*,2S*,4S*,5R*,6S*) 2-amino-4-[3-(2,6-dichlorophenyl)-ureido]-bicyclo-[3.1.0]hexane-2,6-dicarboxylic acid

A 0° C. solution of the product of example 13a (0.33 g, 0.61 mmol) in EtOAc (35 mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (15 mL total volume) and stirred at room temperature overnight. The reaction mixture was adjusted to pH=7 with 1N HCl and concentrated to dryness. The solids were reconstituted in water, adjusted to pH=12 with 1N NaOH and purified by anion exchange chromatography (Bio-Rad® AG1-X8 resin; acetate form converted to hydroxide form). The product was eluted from the column with 3N AcOH and concentrated to dryness in vacuo to afford crude product. Trituration in 2-propanol:water (5:1) followed by drying under vacuum at 80° C. afforded 0.54 g (0.54 mmol, 89%) of the desired product as a white solid.

mp=>250° C. FDMS: M⁺+1=388. Anal. calcd. for Cl₁₅H₁₅Cl₂N₃O₅.1.3 eq. H₂O: C, 43.77; H, 4.31; N, 10.21. Found: C, 43.55; H, 4.07; N, 10.04.

### EXAMPLE 14

### Synthesis of (1S*,2S*,4S*,5R*,6S*) 2-amino-4-[3-(2,4-dichlorophenyl)ureido]bicyclo(3.1.0]hexane-2,6-dicarboxylic acid

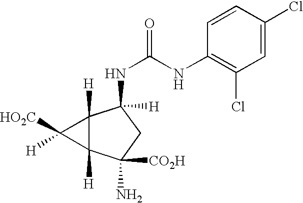

### A. (1S*,2S*,4S*,5R*,6S*) Diethyl 2-(N-t-butyloxycarbonyl-amino)-4-[3-(2,4-dichloro-phenyl)ureido]bicyclo[3.1.0]-hexane-2,6-dicarboxylic acid

A room temperature solution of the product of preparation 1 (0.25 g, 0.70 mmol) in CH₂Cl₂ (10 mL) was treated in one portion with 2,4-dichlorophenyl isocyanate (0.16 g, 0.85 mmol). The resulting reaction mixture was allowed to stir at room temperature overnight. The reaction mixture was concentrated in vacuo and purified by SiO₂ chromatography (PC-TLC: 10% EtOAc/hexanes to 50% EtOAc/hexanes) to yield 0.36 g (0.66 mmol, 94%) of a white foam.

FDMS: M−1=542. Anal. calcd. for C₂₄H₃₁Cl₂N₃O₇.0.1 eq. CH₂Cl₂: C, 52.35; H, 5.69; N, 7.60. Found: C, 52.73; H, 5.76; N, 7.48.

### B. (1S*,2S*,4S*,5R*,6S*) 2-amino-4-[3-(2,4-dichlorophenyl)ureido]bicyclo-[3.1.0]hexane-2,4-dicarboxylic acid

A 0° C. solution of the product of example 14a (0.34 g, 0.62 mmol) in EtOAc (35 mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (15 mL total volume) and stirred at room temperature overnight. The reaction mixture was adjusted to pH=7 with 1N HCl and concentrated to dryness. The solids were reconstituted in water, adjusted to pH=12 with 1N NaOH and purified by anion exchange chromatography (Bio-Rad® AG1-X8 resin; acetate form converted to hydroxide form). The product was eluted from the column with 3N AcOH and concentrated to dryness in vacuo to afford crude product. Trituration in 2-propanol:water (5:1) followed by drying under vacuum at 80° C. afforded 0.13 g (0.33 mmol, 53%) of the desired product as a white solid.

mp=>250° C. FDMS: M−1=388. Anal. calcd. for C₁₅H₁₅Cl₂N₃O₅: C, 46.41; H, 3.89; N, 10.82. Found: C, 43.88; H, 3.87; N, 9.80.

### EXAMPLE 15

### Synthesis of (1S*,2S*,4S*,5R*,6S*) 2-amino-4-[3-(3,5-dichlorophenyl)ureido]bicyclo[3.1.0]hexane-2,6-dicarboxylic acid

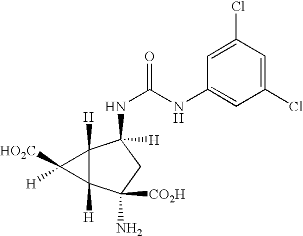

### A. (1S*,2S*,4S*,5R*,6S*) Diethyl 2-(N-t-butyloxycarbonyl-amino)-4-[3-(3,5-dichloro-phenyl)ureido]bicyclo[3.1.0]-hexane-2,6-dicarboxylic acid

A room temperature solution of the product of preparation 1 (0.25 g, 0.70 mmol) in CH₂Cl₂ (10 mL) was treated in one portion with 3,5-dichlorophenyl isocyanate (0.17 g, 0.88 mmol). The resulting reaction mixture was allowed to stir at room temperature overnight. The reaction mixture was concentrated in vacuo and purified by SiO₂ chromatography (PC-TLC: 10% EtOAc/hexanes to 50% EtOAc/hexanes) to yield 0.38 g (0.70 mmol, 99%) of a white foam.

FDMS: M−1=542. Anal. calcd. for C₂₄H₃₁Cl₂N₃O₇.0.1 eq. CH₂Cl₂: C, 52.35; H, 5.69; N, 7.60. Found: C, 52.43; H, 5.69; N, 7.45.

### B. (1S*,2S*,4S*,5R*,6S*) 2-amino-4-[3-(3,5-dichlorophenyl)ureido]bicyclo-[3.1.0]hexane-3,5-dicarboxylic acid

A 0° C. solution of the product of example 15a (0.35 g, 0.64 mmol) in EtOAc (35 mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (15 mL total volume) and stirred at room temperature overnight. The reaction mixture was adjusted to pH=7 with 1N HCl and concentrated to dryness. The solids were reconstituted in water, adjusted to pH=12 with 1N NaOH and purified by anion exchange chromatography (Bio-Rad® AG1-X8 resin; acetate form converted to hydroxide form). The product was eluted from the column with 3N AcOH and concentrated to dryness in vacuo to afford crude product. Trituration in methanol followed by drying under vacuum at 80° C. afforded 0.15 g (0.39 mmol, 61%) of the desired product as a white solid.

mp=>250° C. FDMS: M−2=386. Anal. calcd. for C₁₅H₁₅Cl₂N₃O₅.0.5 eq. H₂O: C, 45.36; H, 4.06; N, 10.58. Found: C, 45.11; H, 3.69; N, 10.88.

### EXAMPLE 16

### Synthesis of (1S*,2S*_{,}4S*,5R*,6S*) 2-amino-4-[3-(3,4-dichlorophenyl)ureido]bicyclo[3.1.0]hexane-2,6-dicarboxylic acid

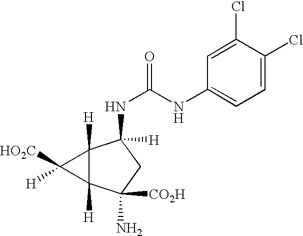

### A. (1S*,2S*,4S*,5R*,6S*) Diethyl 2-(N-t-butyloxycarbonyl-amino)-4-[3-(3,4-dichloro-phenyl)ureido]bicyclo[3.1.0]-hexane-2,6-dicarboxylic acid

A room temperature solution of the product of preparation 1 (0.25 g, 0.70 mmol) in CH₂Cl₂ (10 mL) was treated in one portion with 3,4-dichlorophenyl isocyanate (0.17 g, 0.88 mmol). The resulting reaction mixture was allowed to stir at room temperature overnight. The reaction mixture was concentrated in vacuo and purified by SiO₂ chromatography (PC-TLC: 10% EtOAc/hexanes to 50% EtOAc/hexanes) to yield 0.36 g (0.66 mmol, 94%) of a white foam.

FDMS: M−1=542. Anal. calcd. for C₂₄H₃₁Cl₂N₃O₇.0.1 eq. CH₂Cl₂: C, 52.35; H, 5.69; N, 7.60. Found: C, 52.26; H, 5.59; N, 7.34.

A 0° C. solution of the product of example 16a (0.33 g, 0.61 mmol) in EtOAc (35 mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (15 mL total volume) and stirred at room temperature overnight. The reaction mixture was adjusted to pH=7 with 1N HCl and concentrated to dryness. The solids were reconstituted in water, adjusted to pH=12 with 1N NaOH and purified by anion exchange chromatography (Bio-Rad® AG1-X8 resin; acetate form converted to hydroxide form). The product was eluted from the column with 3N AcOH and concentrated to dryness in vacuo to afford crude product. Trituration in methanol followed by drying under vacuum at 80° C. afforded 0.12 g (0.31 mmol, 51%) of the desired product as a white solid.

mp=>250° C. FDMS: M−2=386. Anal. calcd. for C₁₅H₁₅Cl₂N₃O₅.0.2 eq. H₂O: C, 45.98; H, 3.96; N, 10.73. Found: C, 45.73; H, 3.87; N, 10.42.

### EXAMPLE 17

### Synthesis of (1S*,2S*,4S*,5R*,6S*) 2-amino-4-[3-(2-naphthyl)ureido]bicyclo[3.1.0]hexane-2,6-dicarboxylic acid

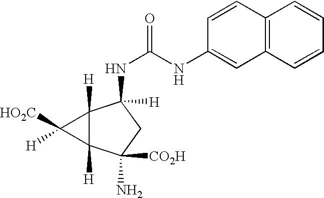

### A. (1S*,2S*,4S*,5R*,6S*) Diethyl 2-(N-t-butyloxycarbonyl-amino)-4-[3-(2-naphthyl)-ureido]bicyclo[3.1.0]hexane-2,6-dicarboxylic acid

A room temperature solution of the product of preparation 1 (0.25 g, 0.70 mmol) in CH₂Cl₂ (10 mL) was treated in one portion with 2-naphthyl isocyanate (0.15 g, 0.88 mmol). The resulting reaction mixture was allowed to stir at room temperature overnight. The reaction mixture was concentrated in vacuo and purified by SiO₂ chromatography (PC-TLC: 10% EtOAc/hexanes to 50% EtOAc/hexanes) to yield 0.36 g (0.68 mmol, 98%) of a white foam.

FDMS: M⁺+35 (Cl)=561.

### B. (1S*,2S*,4S*,5R*,6S*) 2-amino-4-[3-(2-naphthyl)ureido]-bicyclo[3.1.0]hexane-2,6-dicarboxylic acid

A 0° C. solution of the product of example 18a (0.35 g, 0.67 mmol) in EtOAc (35 mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (15 mL total volume) and stirred at room temperature overnight. The reaction mixture was adjusted to pH=7 with 1N HCl and concentrated to dryness. The solids were reconstituted in water, adjusted to pH=12 with 1N NaOH and purified by anion exchange chromatography (Bio-Rad® AG1-X8 resin; acetate form converted to hydroxide form). The product was eluted from the column with 3N AcOH and concentrated to dryness in vacuo to afford crude product. Trituration in acetone followed by drying under vacuum at 80° C. afforded 0.11 g (0.30 mmol, 43%) of the desired product as a white solid.

mp=charred>250° C. FDMS: M⁺+1=370. Anal. calcd. for C₁₉H₁₉N₃O₅.0.6 eq. AcOH: C, 59.85; H, 5.32; N, 10.37. Found: C, 59.74; H, 5.34; N, 10.54.

### EXAMPLE 18

### Synthesis of (1S*,2S*,4S*,5R*,6S*) 2-amino-4-[3-(1-naphthyl)ureido]bicyclo[3.1.0]hexane-2,6-dicarboxylic acid

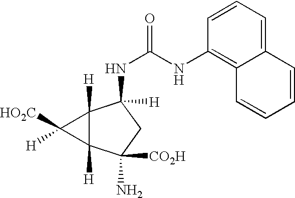

### A. (1S*,2S*,4S*,5R*,6S*) Diethyl 2-(N-t-butyloxycarbonyl-amino)-4-[3-(1-naphthyl)-ureido]bicyclo[3.1.0]hexane-2,6-dicarboxylic acid

A room temperature solution of the product of preparation 1 (0.25 g, 0.70 mmol) in CH₂Cl₂ (10 mL) was treated in one portion with 1-naphthyl isocyanate (0.15 g, 0.88 mmol). The resulting reaction mixture was allowed to stir at room temperature overnight. The reaction mixture was filtered to remove insolubles, concentrated in vacuo and purified by SiO₂ chromatography (PC-TLC: 10% EtOAc/hexanes to 50% EtOAc/hexanes) to yield 0.36 g (0.68 mmol, 98%) of a white foam.

FDMS: M⁺+1=526. Anal. calcd. for C₂₈H₃₅N₃O₇: C, 63.99; H, 6.71; N, 7.99. Found: C, 63.75; H, 6.72; N, 7.93.

### B. (1S*,2S*,4S*,5R*,6S*) 2-amino-4-[3-(1-naphthyl)ureido]-bicyclo[3.1.0]hexane-2,6-dicarboxylic acid

A 0° C. solution of the product of example 19a (0.31 g, 0.60 mmol) in EtOAc (35 mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (15 mL total volume) and stirred at room temperature overnight. The reaction mixture was adjusted to pH=7 with 1N HCl and concentrated to dryness. The solids were reconstituted in water, adjusted to pH=12 with 1N NaOH and purified by anion exchange chromatography (Bio-Rad® AG1-X8 resin; acetate form converted to hydroxide form). The product was eluted from the column with 3N AcOH and concentrated to dryness in vacuo to afford crude product. Trituration in 2-propanol/H₂O (9:1) followed by drying under vacuum at 80° C. afforded 0.12 g (0.32 mmol, 45%) of the desired product as a white solid.

mp =>250° C. FDMS: M⁺+1=370. Anal. calcd. for C₁₉H₁₉N₃O₅.1.0 eq. AcOH.0.3 eq. H₂O: C, 58.00; H, 5.47; N, 9.66. Found: C, 57.81; H, 5.09; N, 9.95.

### EXAMPLE 19

### Synthesis of (1S*, 2S*, 4S*, 5R*, 6S*) 2-Amino-4-(3-phenyl-ureido)-bicyclo[3.1.0]hexane-2,6-dicarboxylic acid

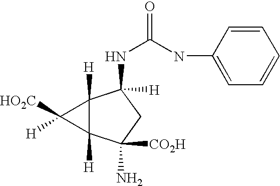

The title compound was prepared utilizing the two step procedure in Example 1. In step (a) the product of Preparation 1 (0.34 g, 0.95 mmol) and phenyl isocyanate (0.12 g, 1.0 mmol) were reacted to afford (1S*, 2S*, 4S*, 5R*, 6S*)-2-tert-butoxycarbonylamino-4-(3-phenyl-ureido)-bicyclo[3.1.0]hexane-2,6-dicarboxylic acid diethyl ester in 88.2% yield. In step (b) the intermediate urea (0.4 g, 0.84 mmol) was converted to the title compound and isolated as a solid in a 69% yield (0.186 g, 0.58 mmol) by cation exchange chromatography (Dowek 50X8-100: 10% Pyridine/H₂O).

m.p.>250□ C. FDMS: M⁺+1=320.

### EXAMPLE 20

### Synthesis of (1S*, 2S*, 4S*, 5R*, 6S*) 2-Amino-4-(3-methyl-ureido)-bicyclo[3.1.0]hexane-2,6-dicarboxylic acid

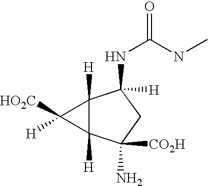

The title compound was prepared utilizing the two step procedure in Example 1. In step (a) the product of Preparation 1 (0.25 g, 0.7 mmol) and methyl isocyanate (0.044 g, 0.77 mmol) were reacted to afford (1S*, 2S*, 4S*, 5R*, 6S*)-2-tert-butoxycarbonylamino-4-(3-methyl-ureido)-bicyclo[3.1.0)hexane-2,6-dicarboxylic acid diethyl ester in 73.7% yield. In step (b) the intermediate urea (0.2 g, 0.45 mmol) was converted to the title compound and isolated as a solid in a 95% yield (0.11 g, 0.43 mmol) by cation exchange chromatography (Dowex 50X8-100: 10% Pyridine/H₂O).

m.p.>250□ C. FDMS: M⁺+1=320.

### EXAMPLE 21

### Synthesis of (1S*,2S*,4S*,5R*,6S*) 2-Amino-4-(3-phenylthioureido)-bicyclo[3.1.0]hexane-2,6-dicarboxylic acid

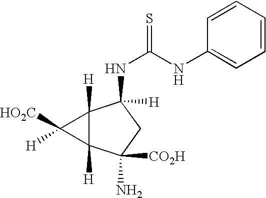

### A. (1S*,2S*,4S*,5R*,6S*) Diethyl 2-(N-t-butyloxycarbonyl-amino)-4-[3-phenylthioureido]bicyclo[3.1.0]hexane-2,6-dicarboxylic acid

A room temperature solution of the product of preparation 1 (0.25 g, 0.70 mmol) in CH₂Cl₂ (10 mL) was treated in one portion with phenyl isothiocyanate (0.14 g, 1.0 mmol). The resulting reaction mixture was allowed to stir at room temperature overnight. The reaction mixture was concentrated in vacuo and purified by SiO₂ chromatography (PC-TLC: 10% EtOAc/hexanes to 50% EtOAc/hexanes) to yield 0.28 g (0.57 mmol, 81%) of a white foam.

FDMS: M−1=490. Anal. calcd. for C₂₄H₃₃N₃O₆S.0.1 eq. CH₂Cl₂: C, 57.88; H, 6.69; N, 8.40. Found: C, 57.93; H, 6.52; N, 8.34.

### B. (1S*,2S*,4S*,5R*,6S*) 2-amino-4-[3-(1-naphthyl)ureido]-bicyclo[3.1.0]hexane-2,6-dicarboxylic acid

A 0° C. solution of the product of example 19a (0.31 g, 0.60 mmol) in EtOAc (35 mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (15 mL total volume) and stirred at room temperature overnight. The reaction mixture was adjusted to pH=7 with 1N HCl and concentrated to dryness. The solids were reconstituted in water, adjusted to pH=2 and the resulting precipitate collected via vacuum filtration to yield the title compound as the HCl salt. Subsequent drying under vacuum at 80° C. afforded 0.14 g (0.38 mmol, 71%) of the desired product as a white solid.

mp=>275° C. FDMS: M⁺+1=336 (free base). Anal. calcd. for C₁₅H₁₇N₃O₄S.1.0 eq. HCl: C, 48.45; H, 4.88; N, 11.30. Found: C, 48.58; H, 4.52; N, 11.17.

### EXAMPLE 22

### Synthesis of (1S*, 2S*, 4S*, 5R*, 6S*) 2-Amino-4-[(thiophene-3-carbonyl)-amino]-bicyclo[3.1.0]hexane-2,6-dicarboxylic acid

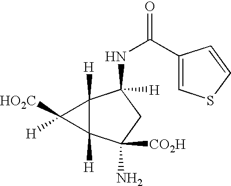

### A. (1S*, 2S*, 4S*, 5R*, 6S*) 2-tert-Butoxycarbonylamino-4-[(thiophene-3-carbonyl)-amino]-bicyclo[3.1.0]hexane-2,6-dicarboxylic acid diethyl ester

3-Thiophenecarboxylic acid was dissolved in 3 ml of anhydrous DMF and cooled to 0□ C. under a nitrogen atmosphere. 1,3-Dicyclohexylcarbodiimide (0.133 g, 1.05 mmol) and hydroxybenztriazole (0.19 g, 1.4 mmol) were added sequentially to the acid. The reaction was allowed to warm to ambient temperature for 15 minutes, then cooled to 0□ C. again. The product of Preparation 1 (0.25 g, 0.7 mmol) in 2 ml of anhydrous DMF was added to the cooled reaction mixture above and allowed to stir over night. Partitioned the reaction between EtOAc and H₂O, washed with brine and the organics dried over MgSO₄. The isolated crude material was triturated with EtOAc and the solids were filtered. The product was purified using PT-TLC (10% EtOAc/hexanes to 50% EtOAc/hexanes) to give 100% yield (0.33 g, 0.7 mmol).

FDMS: M⁺−1=465.

### B. (1S*, 2S*, 4S*, 5R*, 6S*) 2-Amino-4-[(thiophene-3-carbonyl)-amino]-bicyclo[3.1.0]hexane-2,6-dicarboxylic acid

A solution of the product from step (a) (0.34 g, 0.73 mmol), in EtOAc was chilled to 0□ C. The reaction solution was saturated with HCl (g) and allowed to react until the Boc group was cleaved as indicated by TLC. The reaction was concentrated under vacuum and rediluted in THF (10 ml). 1N NaOH (10 ml) was added and the mixture was allowed to stir overnight (pH=14). The reaction mixture was titrated with 1N HCl to a pH=−7 and organics were removed under vacuum. The title compound (0.118 g, 0.38 mmol) was isolated in 52.1% yield by isoelectric precipitation at pH=˜3-4.

m.p.>250□ C. FDMS: M⁺+1=311. Anal. calcd. For C₁₃H₁₄N₂O₅S.0.2 H₂O: C, 49.74; H, 4.62; N, 8.92. Found: C, 49.70; H, 4.59; N, 8.90.

### EXAMPLE 23

### Synthesis of (1S*, 2S*, 4S*, 5R*, 6S*) 4-Acetylamino-2-amino-bicyclo[3.1.0]hexane-2,6-dicarboxylic acid

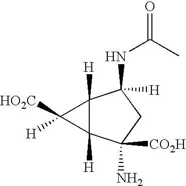

The title compound was prepared utilizing the two step procedure in Example 22. In step (a) the product of Preparation 1 (0.2 g, 0.56 mmol) and acetyl chloride (0.053 g, 0.67 mmol) were reacted to afford (1S*, 2S*, 4S*, 5R*, 6S*)-4-acetylamino-2-tert-butoxycarbonylamino-bicyclo[3.1.0]hexane-2,6-dicarboxylic acid diethyl ester in 99% yield. In step (b) the intermediate amide (0.19 g, 0.48 mmol) was converted to the title compound in a 61.9% yield (0.072 g, 0.3 mmol).

m.p.=223-224□ C. FDMS: M⁺−1=241. Anal. calcd. For C₁₀H₁₄N₂O₅.1.5 H₂O: C, 44.61; H, 6.36; N, 10.40. Found: C, 44.67; H, 6.28; N, 10.80.

### EXAMPLE 24

### Synthesis of (1S*, 2S*, 4S*, 5R*, 6S*) 2-Amino-4-benzoylamino-bicyclo[3.1.0]hexane-2,6-dicarboxylic acid

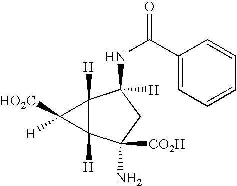

The title compound was prepared utilizing the two step procedure in Example 22. In step (a) the product of Preparation 1 (0.32 g, 0.8 mmol) and benzoyl chloride (0.15 g, 1.0 mmol) were reacted to afford (1S*, 2S*, 4S*, 5R*, 6S*)-4-benzoylamino-2-tert-butoxycarbonylamino-bicyclo[3.1.0)hexane-2,6-dicarboxylic acid diethyl ester in 99% yield. In step (b) the intermediate amide (0.39 g, 0.85 mmol) was converted to the title compound in a 46.6% yield (0.12 g, 0.39 mmol).

m.p.=238-240□ C. FDMS: M⁺+1=305.

### EXAMPLE 25

### Synthesis of (1S*, 2S*, 4S*, 5R*, 6S*) 2-Amino-4-(4-chloro-benzoylamino)-bicyclo[3.1.0]hexane-2,6-dicarboxylic acid

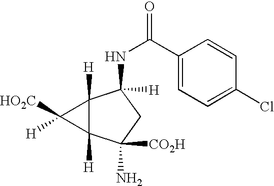

The title compound was prepared utilizing the two step procedure in Example 22. In step (a) the product of Preparation 1 (0.34 g, 0.95 mmol) and 4-chlorobenzoyl chloride (0.2 g, 1.15 mmol) were reacted to afford (1S*, 2S*, 4S*, 5R*, 6S*)-2-tert-butoxycarbonylamino-4-(4-chloro-benzoylamino)-bicyclo[3.1.0]hexane-2,6-dicarboxylic acid diethyl ester in 86.9% yield. In step (b) the intermediate amide (0.4 g, 0.81 mmol) was converted to the title compound and isolated as a white solid in a 87.5% yield (0.24 g, 0.71 mmol) by isoelectric precipitation (pH=˜3-4).

m.p.>250□ C. FDMS: M⁺+1=339.

### EXAMPLE 26

### Synthesis of (1S*, 2S*, 4S*, 5R*, 6S*) 2-Amino-4-(3-chloro-benzoylamino)-bicyclo[3.1.0]hexane-2,6-dicarboxylic acid

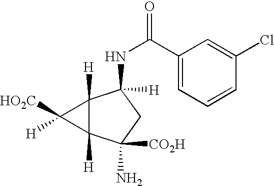

The title compound was prepared utilizing the two step procedure in Example 22. In step (a) the product of Preparation 1 (0.33 g, 0.93 mmol) and 3-chlorobenzoyl chloride (0.19 g, 1.11 mmol) were reacted to afford (1S*, 2S*, 4S*, 5R*, 6S*)-2-tert-butoxycarbonylamino-4-(3-chloro-benzoylamino)-bicyclo[3.1.0]hexane-2,6-dicarboxylic acid diethyl ester in 99% yield. In step (b) the intermediate amide (0.45 g, 0.91 mmol) was converted to the title compound and isolated as a white solid in a 67.6% yield (0.208 g, 0.61 mmol) by isoelectric precipitation (pH=˜3-4).

m.p.=254-255□ C. FDMS: M⁺+1=339. Anal. calcd. For C₁₅H₁₅N₂O₅Cl.0.1 H₂O: C, 52.90; H, 4.50; N, 8.23. Found: C, 52.88; H, 4.40; N, 8.16.

### EXAMPLE 27

### Synthesis of (1S*, 2S*, 4S*, 5R*, 6S*) 2-Amino-4-(4-methoxy-benzoylamino)-bicyclo[3.1.0]hexane-2,6-dicarboxylic acid

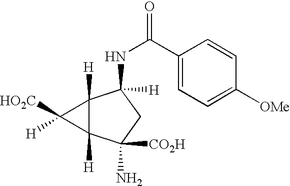

The title compound was prepared utilizing the two step procedure in Example 22. In step (a) the product of Preparation 1 (0.3 g, 0.84 mmol) and 3-methoxybenzoyl chloride (0.17 g, 1.01 mmol) were reacted to afford (1S*, 2S*, 4S*, 5R*, 6S*)-2-tert-butoxycarbonylamino-4-(4-methoxy-benzoylamino)-bicyclo[3.1.0]hexane-2,6-dicarboxylic acid diethyl ester in 77.7% yield. In step (b) the intermediate amide (0.3 g, 0.61 mmol) was converted to the title compound in a 64.6% yield (0.132 g, 0.4 mmol).

m.p.=248-249□ C. FDMS: M⁺+1=335. Anal. calcd. For C₁₆H₁₈N₂O₆.0.3 H₂O: C, 56.57; H, 5.52; N, 8.25. Found: C, 56.67; H, 5.25; N, 8.22.

### EXAMPLE 28

### Synthesis of (1S*, 2S*, 4S*, 5R*, 6S*) 2-Amino-4-[(naphthalene-1-carbonyl)-amino]-bicyclo[3.1.0]hexane-2,6-dicarboxylic acid

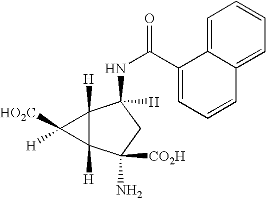

The title compound was prepared utilizing the two step procedure in Example 22. In step (a) the product of Preparation 1 (0.33 g, 0.93 mmol) and 1-naphthoy chloride (0.212 g, 1.1 mmol) were reacted to afford (1S*, 2S*, 4S*, 5R*, 6S*)-2-tert-butoxycarbonylamino-4-[(naphthalene-1-carbonyl)-amino]-bicyclo[3.1.0]hexane-2,6-dicarboxylic acid diethyl ester in 99% yield. In step (b) the intermediate amide (0.39 g, 0.76 mmol) was converted to the title compound in a 51.7% yield (0.14 g, 0.4 mmol).

m.p.=234-235□ C. FDMS: M⁺+1=355.

### EXAMPLE 29

### Synthesis of (1S*, 2S*, 4S*, 5R*, 6S*) 2-Amino-4-[(naphthalene-2-carbonyl)-amino)-bicyclo[3.1.0]hexane-2,6-dicarboxylic acid

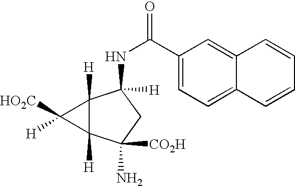

The title compound was prepared utilizing the two step procedure in Example 22. In step (a) the product of Preparation 1 (0.33 g, 0.93 mmol) and 2-naphthoy chloride (0.21 g, 1.1 mmol) were reacted to afford (1S*, 2S*, 4S*, 5R*, 6S*)-2-tert-butoxycarbonylamino-4-[(naphthalene-2-carbonyl)-amino]-bicyclo[3.1.0]hexane-2,6-dicarboxylic acid diethyl ester in 99% yield. In step (b) the intermediate amide (0.43 g, 0.84 mmol) was converted to the title compound and isolated as a white solid in an 88.7% yield (0.264 g, 0.745 mmol) by isoelectric precipitation (pH=˜3-4).

m.p.=257-258□ C. FDMS: M⁺+1=355. Anal. calcd. For C₁₉H₁₈N₂O₅.0.2 H₂O: C, 63.75; H, 5.80; N, 7.83. Found: C, 63.79; H, 4.96; N, 7.78.

### EXAMPLE 30

### Synthesis of (1S*, 2S*, 4S*, 5R*, 6S*) 2-Amino-4-(4-fluoro-benzoylamino)-bicyclo[3.1.0]hexane-2,6-dicarboxylic acid

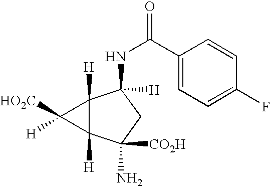

The title compound was prepared utilizing the two step procedure in Example 22. In step (a) the product of Preparation 1 (0.15 g, 0.42 mmol) and 4-fluorobenzoyl chloride (0.08 g, 0.5 mmol) were reacted to afford (1S*, 2S*, 4S*, 5R*, 6S*)-2-tert-butoxycarbonylamino-4-(4-fluoro-benzoylamino)-bicyclo[3.1.0]hexane-2,6-dicarboxylic acid diethyl ester in 89.4% yield. In step (b) the intermediate amide (0.15 g, 0.31 mmol) was converted to the title compound and isolated as a solid in a 77.4% yield (0.077 g, 0.24 mmol) by isoelectric precipitation (pH=˜3-4).

m.p.>250□ C. FDMS: M⁺+1=323. Anal. calcd. For C₁₅H₁₅N₂O₅F.0.5 H₂O: C, 54.38; H, 4.87; N, 8.46. Found: C, 54.46; H, 4.61; N, 8.41.

### EXAMPLE 31

### Synthesis of (1S*, 2S*, 4S*, 5R*, 6S*) 2-Amino-4-[(thiophene-2-carbonyl)-amino]-bicyclo[3.1.0]hexane-2,6-dicarboxylic acid

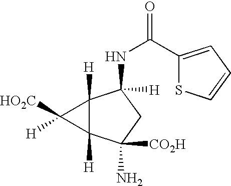

The title compound was prepared utilizing the two step procedure in Example 22. In step (a) the product of Preparation 1 (0.15 g, 0.42 mmol) and 2-thiophenecarbonyl chloride (0.074 g, 0.51 mmol) were reacted to afford (1S*, 2S*, 4S*, 5R*, 6S*)-2-tert-butoxycarbonylamino-4-[(thiophene-2-carbonyl)-amino]-bicyclo[3.1.0]hexane-2,6-dicarboxylic acid diethyl ester in 79% yield. In step (b) the intermediate amide (0.14 g, 0.3 mmol) was converted to the title compound in a 49.4% yield (0.046 g, 0.16 mmol).

m.p.=254-255□ C. FDMS: M⁺=310. Anal. calcd. For C₁₃H₁₄N₂O₅S.2.0 H₂O: C, 45.08; H, 5.24; N, 8.09. Found: C, 45.05; H, 5.00; N, 8.09.

### EXAMPLE 32

### Synthesis of (1S*, 2S*, 4S*, 5R*, 6S*) 2-Amino-4-(2-methoxy-benzoylamino)-bicyclo[3.1.0]hexane-2,6-dicarboxylic acid

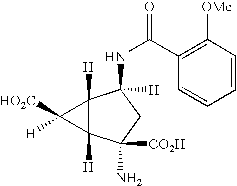

The title compound was prepared utilizing the two step procedure in Example 22. In step (a) the product of Preparation 1 (0.16 g, 0.45 mmol) and 2-methoxybenzoyl chloride (0.092 g, 0.54 mmol) were reacted to afford (1S*, 2S*, 4S*, 5R*, 6S*)-2-tert-butoxycarbonylamino-4-(2-methoxy-benzoylamino)-bicyclo[3.1.0]hexane-2,6-dicarboxylic acid diethyl ester in 86.1% yield. In step (b) the intermediate amide (0.15 g, 0.31 mmol) was converted to the title compound in a 40.9% yield (0.045 g, 0.14 mmol).

m.p.=247-248□ C. FDMS: M⁺+1=335. Anal. calcd. For C₁₆H₁₈N₂O₆.0.7 H₂O: C, 55.39; H, 5.64; N, 8.08. Found: C, 55.17; H, 5.29; N, 8.00.

### EXAMPLE 33

### Synthesis of (1S*, 2S*, 4S*, 5R*, 6S*) 2-Amino-4-(3-methoxy-3.1.0]hexane-2,6-dicarboxylic acid

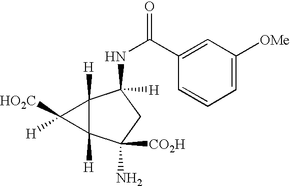

The title compound was prepared utilizing the two step procedure in Example 22. In step (a) the product of Preparation 1 (0.16 g, 0.45 mmol) and 3-methoxybenzoyl chloride (0.092 g, 0.54 mmol) were reacted to afford (1S*, 2S*, 4S*, 5R*, 6S*)-2-tert-butoxycarbonylamino-4-(3-methoxy-benzoylamino)-bicyclo[3.1.0]hexane-2,6-dicarboxylic acid diethyl ester in 95.1% yield. In step (b) the intermediate amide (0.19 g, 0.39 mmol) was converted to the title compound in a 54.1% yield (0.070 g, 0.21 mmol).

m.p.=235-236□ C. FDMS: M⁺+1=335. Anal. calcd. For C₁₆H₁₈N₂O₆.1.2 H₂O: C, 53.99; H, 5.78; N, 7.87. Found: C, 54.01; H, 5.45; N, 7.87.

### EXAMPLE 34

### Synthesis of (1S*, 2S*, 4S*, 5R*, 6S*) 2-Amino-4-(3-methyl-benzoylamino)-bicyclo[3.1.0]hexane-2,6-dicarboxylic acid

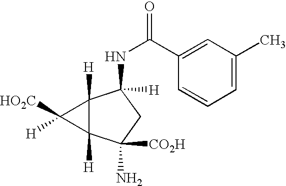

The title compound was prepared utilizing the two step procedure in Example 22. In step (a) the product of Preparation 1 (0.2 g, 0.56 mmol) and m-toluoyl chloride (0.104 g, 0.67 mmol) were reacted to afford (1S*, 2S*, 4S*, 5R*, 6S*)-2-tert-butoxycarbonylamino-4-(3-methyl-benzoylamino)-bicyclo[3.1.0]hexane-2,6-dicarboxylic acid diethyl ester in 86.5% yield. In step (b) the intermediate amide (0.2 g, 0.42 mmol) was converted to the title compound in a 44.9% yield (0.060 g, 0.19 mmol).

m.p.>250□ C. FDMS: M⁺+1=319. Anal. calcd. For C₁₆H₁₈N₂O₅.0.2 H₂O: C, 59.69; H, 5.76; N, 8.70. Found: C, 59.55; H, 5.53; N, 8.53.

### EXAMPLE 35

### Synthesis of (1S*, 2S*, 4S*, 5R*, 6S*) 2-Amino-4-(3-trifluoromethyl-benzoylamino)-bicyclo[3.1.0]hexane-2,6-dicarboxylic acid

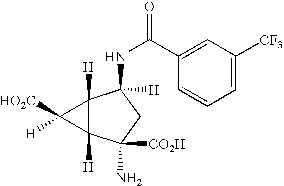

The title compound was prepared utilizing the two step procedure in Example 1. In step (a) the product of Preparation 1 (0.25 g, 0.7 mmol) and 3-(trifluoromethyl)benzoyl chloride (0.175 g, 0.84 mmol) was reacted to afford (1S*, 2S*, 4S*, 5R*, 6S*)-2-tert-butoxycarbonylamino-4-(3-trifluoromethyl-benzoylamino)-bicyclo[3.1.0]hexane-2,6-dicarboxylic acid diethyl ester in 99% yield. In step (b) the intermediate amide (0.26 g, 0.49 mmol) was converted to the title compound and isolated as a solid in a 67.4% yield (0.123 g, 0.33 mmol) by isoelectric precipitation (pH=3-4).

m.p.=249-250□ C. FDMS: M⁺+1=373. Anal. calcd. For C₁₆H₁₅N₂O₅F₃.0.2 H₂O: C, 51.12; H, 4.13; N, 7.45. Found: C, 50.94; H, 3.99; N, 7.41.

### EXAMPLE 36

### Synthesis of (1S*, 2S*, 4S*, 5R*, 6S*) 2-Amino-4-(3-fluoro-benzoylamino)-bicyclo[3.1.0]hexane-2,6-dicarboxylic acid

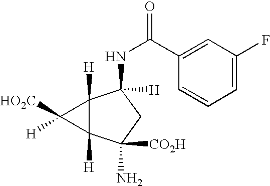

The title compound was prepared utilizing the two step procedure in Example 22. In step (a) the product of Preparation 1 (0.23 g, 0.65 mmol) and 3-fluorobenzoyl chloride (0.1 g, 0.77 mmol) were reacted to afford (1S*, 2S*, 4S*, 5R*, 6S*)-2-tert-butoxycarbonylamino-4-(3-fluoro-benzoylamino)-bicyclo[3.1.0]hexane-2,6-dicarboxylic acid diethyl ester 80% yield. In step (b) the intermediate amide (0.22 g, 0.46 mmol) was converted to the title compound and isolated as a solid in a 58.0% yield (0.086 g, 0.27 mmol) by isoelectric precipitation (pH=˜3-4).

m.p.>250□ C. FDMS: M⁺−1=321. Anal. calcd. For C₁₅H₁₅N₂O₅F.0.3 H₂O: C, 54.98; H, 4.80; N, 8.55. Found: C, 54.92; H, 4.63; N, 8.48.

### EXAMPLE 37

### Synthesis of (1S*, 2S*, 4S*, 5R*, 6S*) 2-Amino-4-(3,4-dichloro-benzoylamino)-bicyclo[3.1.0]hexane-2,6-dicarboxylic acid

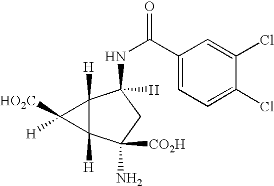

The title compound was prepared utilizing the two step procedure in Example 22. In step (a) the product of Preparation 1 (0.26 g, 0.73 mmol) and 3,4-dichlorobenzoyl chloride (0.183 g, 0.88 mmol) were reacted to afford (1S*, 2S*, 4S*, 5R*, 6S*)-2-tert-butoxycarbonylamino-4-(3,4-dichloro-benzoylamino)-bicyclo[3.1.0]hexane-2,6-dicarboxylic acid diethyl ester in 95.7% yield. In step (b) the intermediate amide (0.28 g, 0.53 mmol) was converted to the title compound and isolated as a solid in a 73.8% yield (0.146 g, 0.39 mmol) by isoelectric precipitation (pH=˜3-4).

m.p.>250□ C. FDMS: M⁺=373. Anal. calcd. For C₁₅H₁₄N₂O₅Cl₂.0.09 C₃H₈O.: C, 48.44; H, 3.91; N, 7.39. Found: C, 48.77; H, 3.51; N, 7.49.

### EXAMPLE 38

### Synthesis of (1S*, 2S*, 4S*, 5R*, 6S*) 2-Amino-4-(3,5-dichloro-benzoylamino)-bicyclo[3.1.0]hexane-2,6-dicarboxylic acid

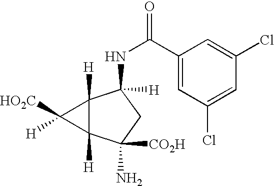

The title compound was prepared utilizing the two step procedure in Example 22. In step (a) the product of Preparation 1 (0.22 g, 0.62 mmol) and 3,5-dichlorobenzoyl chloride (0.155 g, 0.74 mmol) were reacted to afford (1S*, 2S*, 4S*, 5R*, 6S*)-2-tert-Butoxycarbonylamino-4-(3,5-dichloro-benzoylamino)-bicyclo[3.1.0]hexane-2,6-dicarboxylic acid diethyl ester in 91% yield. In step (b) the intermediate amide (0.285 g, 0.54 mmol) was converted to the title compound and isolated as a solid in a 84.6% yield (0.17 g, 0.46 mmol) by isoelectric precipitation (pH=˜3-4).

m.p.=250-251□ C. FDMS: M⁺=373. Anal. calcd. For C₁₅H₁₄N₂O₅Cl₂.: C, 48.28; H, 3.78; N, 7.33. Found: C, 48.02; H, 3.78; N, 7.33.

### EXAMPLE 39

### Synthesis of (1S*, 2S*, 4S*, 5R*, 6S*) 2-Amino-4-(2,4-dichloro-benzoylamino)-bicyclo[3.1.0]hexane-2,6-dicarboxylic acid

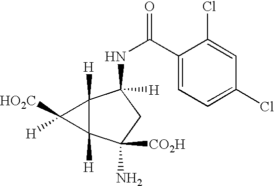

The title compound was prepared utilizing the two step procedure in Example 22. In step (a) the product of Preparation 1 (0.24 g, 0.67 mmol) and 2,4-dichlorobenzoyl chloride (0.169 g, 0.81 mmol) were reacted to afford (1S*, 2S*, 4S*, 5R*, 6S*)-2-tert-butoxycarbonylamino-4-(2,4-dichloro-benzoylamino)-bicyclo[3.1.0]hexane-2,6-dicarboxylic acid diethyl ester in 87% yield. In step (b)the intermediate amide (0.31 g, 0.59 mmol) was converted to the title compound and isolated as a solid in a 67.3% yield (0.147 g, 0.39 mmol) by isoelectric precipitation (pH=˜3-4).

m.p.=231-232□ C. FDMS: M⁺=373. Anal. calcd. For C₁₅H₁₄N₂O₅Cl₂.: C, 45.63; H, 4.19; N, 7.10. Found: C, 45.40; H, 3.96; N, 7.47.

### EXAMPLE 40

### Synthesis of (1S*, 2S*, 4S*, 5R*, 6S*) 2-Amino-4-(2,3-dichloro-benzoylamino)-bicyclo[3.1.0]hexane-2,6-dicarboxylic acid

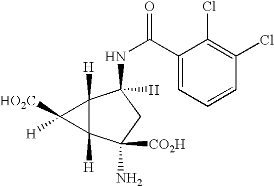

The title compound was prepared utilizing the two step procedure in Example 22. In step (a) the product of Preparation 1 (0.23 g, 0.65 mmol) and 2,3-dichlorobenzoyl chloride (0.162 g, 0.77 mmol) were reacted to afford (1S*, 2S*, 4S*, 5R*, 6S*)-2-tert-Butoxycarbonylamino-4-(2,3-dichloro-benzoylamino)-bicyclo[3.1.0]hexane-2,6-dicarboxylic acid diethyl ester in 81% yield. In step (b) the intermediate amide (0.275 g, 0.52 mmol) was converted to the title compound and isolated as a solid in a 70.6% yield (0.137 g, 0.37 mmol) by isoelectric precipitation (pH=˜3-4).

m.p.=249-250□ C. FDMS: M⁺=373. Anal. calcd. For C₁₅H₁₄N₂O₅Cl₂.1.0 H₂O: C, 46.05; H, 4.12; N, 7.16. Found: C, 46.19; H, 3.92; N, 7.12.

### EXAMPLE 41

### Synthesis of (1S*, 2S*, 4S*, 5R*, 6S*) 2-Amino-4-(2-fluoro-benzoylamino)-bicyclo[3.1.0]hexane-2,6-dicarboxylic acid

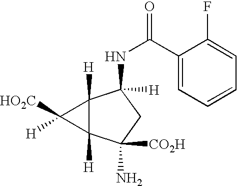

The title compound was prepared utilizing the two step procedure in Example 22. In step (a) the product of Preparation 1 (0.245 g, 0.69 mmol) and 2-fluorobenzoyl chloride (0.131 g, 0.83 mmol) were reacted to afford (1S*, 2S*, 4S*, 5R*, 6S*)-2-tert-butoxycarbonylamino-4-(2-fluoro-benzoylamino)-bicyclo[3.1.0]hexane-2,6-dicarboxylic acid diethyl ester in 78% yield. In step (b) the intermediate amide (0.25 g, 0.52 mmol) was converted to the title compound and isolated as a solid in a 62.1% yield (0.104 g, 0.32 mmol) by isoelectric precipitation (pH=˜3-4).

m.p.=□ C. FDMS: M⁺−1=321. Anal. calcd. For C₁₅H₁₅N₂O₅F.1.4 H₂O: C, 51.84; H, 5.16; N, 8.06. Found: C, 51.61; H, 4.81; N, 8.11.

### EXAMPLE 42

### Synthesis of (1S*, 2S*, 4S*, 5R*, 6S*) 2-Amino-4-[(1H-indole-2-carbonyl)-amino]-bicyclo[3.1.0]hexane-2,6-dicarboxylic acid

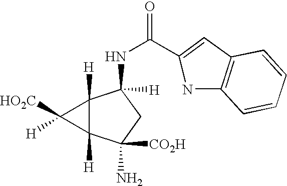

The title compound was prepared utilizing the two step procedure in Example 23. In step (a) the product of Preparation 1 (0.25 g, 0.7 mmol) and 2-indolecarboxylic acid (0.123 g, 0.77 mmol) were reacted to afford (1S*, 2S*, 4S*, 5R*, 6S*)-2-tert-butoxycarbonylamino-4-[(1H-indole-2-carbonyl)-amino]-bicyclo[3.1.0]hexane-2,6-dicarboxylic acid diethyl ester in 91.5% yield. In step (b) the intermediate amide (0.3 g, 0.6 mmol) was converted to the title compound in a 68.9% yield (0.137 g, 0.39 mmol).

m.p.>250□ C. FDMS: M⁺−1=342. Anal. calcd. For C₁₇H₁₇N₃O₅.0.3 H₂O: C, 58.55; H, 5.09; N, 12.05. Found: C, 58.65; H, 4.86; N, 11.88.

### EXAMPLE 43

### Synthesis of (1S*, 2S*, 4S*, 5R*, 6S*) 2-Amino-4-(4-hydroxy-benzoylamino)-bicyclo[3.1.0]hexane-2,6-dicarboxylic acid

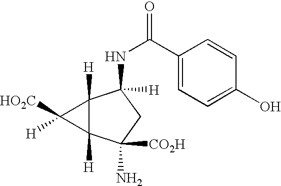

The title compound was prepared utilizing the two step procedure in Example 23. In step (a) the product of Preparation 1 (0.25 g, 0.7 mmol) and 4-hydroxybenzoic acid (0.106 g, 0.77 mmol) were reacted to afford (1S*, 2S*, 4S*, 5R*, 6S*)-2-tert-butoxycarbonylamino-4-(4-hydroxy-benzoylamino)-bicyclo[3.1.0o hexane-2,6-dicarboxylic acid diethyl ester in 93% yield. In step (b) the intermediate amide (0.27 g, 0.56 mmol) was converted to the title compound in a 72.5% yield (0.131 g, 0.41 mmol).

m.p.=252-253□ C. FDMS: M⁺−1=319. Anal. calcd. For C₁₅H₁₆N₂O₆.1.1 H₂O: C, 52.97; H. 5.39; N, 8.24. Found: C, 52.84; H, 5.02; N, 8.20.

### EXAMPLE 44

### Synthesis of (1S*, 2S*, 4S*, 5R*, 6S*) 2-Amino-4-(3-hydroxy-benzoylamino)-bicyclo[3.1.0]hexane-2,6-dicarboxylic acid

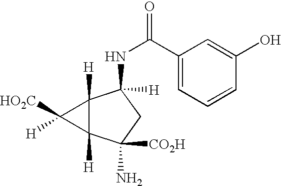

The title compound was prepared utilizing the two step procedure in Example 23. In step (a) the product of Preparation 1 (0.25 g, 0.7 mmol) and 3-hydroxybenzoic acid (0.106 g, 0.77 mmol) were reacted to afford (1S*,2S*, 4S*, 5R*, 6S*)-2-tert-butoxycarbonylamino-4-(3-hydroxy-benzoylamino)-bicyclo[3.1.0)hexane-2,6-dicarboxylic acid diethyl ester in 93% yield. In step (b) the intermediate amide (0.31 g, 0.65 mmol) was converted to the title compound in a 62.9% yield (0.131 g, 0.41 mmol).

m.p.>250□ C. FDMS: M⁺+1=321. Anal. calcd. For C₁₅H₁₆N₂O₆.0.4 H₂O: C, 55.01; H, 5.17; N, 8.55. Found: C, 54.97; H, 5.00; N, 8.52.

### EXAMPLE 45

### Synthesis of (1S*, 2S*, 4S*, 5R*, 6S*) 2-Amino-4-(2-hydroxy-benzoylamino)-bicyclo[3.1.0]hexane-2,6-dicarboxylic acid

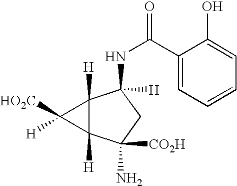

The title compound was prepared utilizing the two step procedure in Example 23. In step (a) the product of Preparation 1 (0.25 g, 0.7 mmol) and 2-hydroxybenzoic acid (0.106 g, 0.77 mmol) were reacted to afford (1S*, 2S*, 4S*, 5R*, 6S*)-2-tert-butoxycarbonylamino-4-(2-hydroxy-benzoylamino)-bicyclo[3.1.0)hexane-2,6-dicarboxylic acid diethyl ester in 76% yield. In step (b) the intermediate amide (0.22 g, 0.46 mmol) was converted to the title compound in a 78.5% yield (0.116 g, 0.36 mmol).

m.p.>250□ C. FDMS: M⁺+1=321. Anal. calcd. For C₁₅H₁₆N₂O₆.0.55 H₂O: C, 54.71; H, 5.20; N, 8.51. Found: C, 54.69; H, 4.88; N, 8.82.

### EXAMPLE 46

### Synthesis of (1S*, 2S*, 4S*, 5R*, 6S*) 2-Amino-4-[(biphenyl-4-carbonyl)-amino]-bicyclo[3.1.0]hexane-2,6-dicarboxylic acid

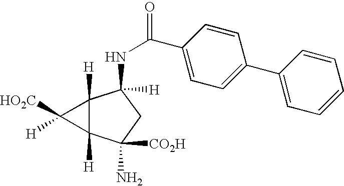

The title compound was prepared utilizing the two step procedure in Example 23. In step (a) the product of Preparation 1 (0.25 g, 0.7 mmol) and 4-biphenylcarboxylic acid (0.153 g, 0.77 mmol) were reacted to afford (1S*, 2S*, 4S*, 5R*, 6S*)-4-[(biphenyl-4-carbonyl)-amino]-2-tert-butoxycarbonylamino-bicyclo[3.1.0]hexane-2,6-dicarboxylic acid diethyl ester in 98.5% yield. In step (b) the intermediate amide (0.36 g, 0.67 mmol) was converted to the title compound in a 76.5% yield (0.195 g, 0.51 mmol).

m.p.=260-261□ C. FDMS: M⁺+1=381. Anal. calcd. For C₂₂H₂₀N₂O₆: C, 66.31; H, 5.30; N, 7.36. Found: C, 66.00; H, 5.40; N, 7.38.

### EXAMPLE 47

### Synthesis of (1S*, 2S*, 4S*, 5R*, 6S*) 2-Amino-4-[(1H-indole-3-carbonyl)-amino]-bicyclo[3.1.0]hexane-2,6-dicarboxylic acid

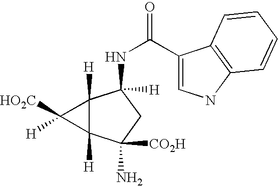

The title compound was prepared utilizing the two step procedure in Example 23. In step (a) the product of Preparation 1 (0.25 g, 0.7 mmol) and indole-3-carboxylic acid (0.124 g, 0.77 mmol) were reacted to afford (1S*, 2S*, 4S*, 5R*, 6S*)-2-tert-butoxycarbonylamino-4-[(1H-indole-3-carbonyl)-amino]-bicyclo[3.1.0]hexane-2,6-dicarboxylic acid diethyl ester in 48.6% yield. In step (b) the intermediate amide (0.16 g, 0.32 mmol) was converted to the title compound in a 91.9% yield (0.101 g, 0.29 mmol).

m.p.=245-246□ C. FDMS: M⁺+1=344. Anal. calcd. For C₁₇H₁₇N₃O₅: C, 56.47; H, 4.88; N, 11.62. Found: C, 56.52; H, 4.85; N, 11.48.

### EXAMPLE 48

### Synthesis of (1S*, 2S*, 4S*, 5R*, 6S*) 2-Amino-4-[(isoquinoline-1-carbonyl)-amino)-bicyclo[3.1.0]hexane-2,6-dicarboxylic acid

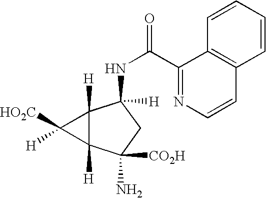

The title compound was prepared utilizing the two step procedure in Example 23. In step (a) the product of Preparation 1 (0.25 g, 0.7 mmol) and 1-isoquinalinecarboxylic acid (0.133 g, 0.77 mmol) were reacted to afford (1S*, 2S*, 4S*, 5R*, 6S*)-2-tert-butoxycarbonylamino-4-[(isoquinoline-1-carbonyl)-amino]-bicyclo[3.1.0]hexane-2,6-dicarboxylic acid diethyl ester 89.4% yield. In step (b) the intermediate amide (0.3 g, 0.59 mmol) was converted to the title compound and isolated as a solid in 56.8% yield (0.119 g, 0.34 mmol) by cation exchange chromatography (Dowex 50X8-100: 10% Pyridine/H₂O).

m.p.=225-226□ C. FDMS: M⁺+1=356. Anal. calcd. For C₁₈H₁₇N₃O₅.0.7 H₂O: C, 58.75; H. 5.04; N, 11.42. Found: C, 58.43; H, 4.73; N, 11.67.

### EXAMPLE 49

### Synthesis of (1S*, 2S*, 4S*, 5R*, 6S*)-2-Amino-4-[(pyridine-2-carbonyl)-amino]-bicyclo[3.1.0]hexane-2,6-dicarboxylic acid

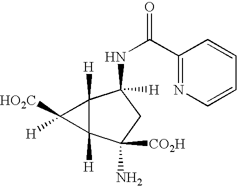

The title compound was prepared by the two step procedure in Example 1. The product of Preparation 1 (0.30 g, 0.84 mmol) was reacted with picolinoyl chloride hydrochloride (0.18 g, 1.02 mmol) in step (a). Purification of the desired intermediate was by PC-TLC (13% EtOAc/Hexane to 100% EtOAc) with a 90% yield. The intermediate, (1S*, 2S*, 4S*, 5R*, 6S*)-2-tert-Butoxycarbonylamino-4-[(pyridine-2-carbonyl)-amino]-bicyclo[3.1.0]hexane-2,6-dicarboxylic acid diethyl ester (0.34 g, 0.74 mmol) was converted to the title compound by step (b) using isoelectric point crystalization from H₂O, pH 3, in a 84% yield (0.19 g, 0.62 mmol).

m.p.=228-229° C. FDMS: M⁺+1=306. Anal. Calcd. For C₁₄H₁₅N₃O₅.0.4 H₂O: C, 53.81; H, 5.10; N, 13.45. Found: C, 53.94; H, 5.10; N, 13.31.

### EXAMPLE 50

### Synthesis of (1S*, 2S*, 4S*, 5R*, 6S*)-2-Amino-4-[(pyridine-3-carbonyl)-amino]-bicyclo[3.1.0]hexane-2,6-dicarboxylic acid

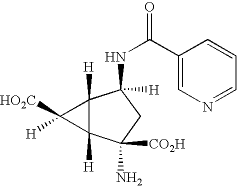

The title compound was prepared by the two step procedure in Example 1. The product of Preparation 1 (0.30 g, 0.84 mmol) was reacted with nicotinoyl chloride hydrochloride (0.18 g, 1.01 mmol) in step (a). Purification of the desired intermediate was by PC-TLC (10% EtOAc/Hexane to 100% EtOAc) with a 90% yield. The intermediate, (1S*, 2S*, 4S*, 5R*, 6S*)-2-tert-Butoxycarbonylamino-4-[pyridine-3-carbonyl)-amino]-bicyclo[3.1.0]hexane-2,6-dicarboxylic acid diethyl ester (0.35 g, 0.76 mmol) was converted into the title compound by step (b) using isoelectric point crystalization from H₂O, pH 3, in a 53% yield (0.12 g, 0.40 mmol).

m.p.=223-224° C. FDMS: M⁺+1=306. Anal. Calcd. For C₁₄H₁₅N₃O₅.1.0 H₂O: C, 52.01; H, 5.30; N, 13.00. Found: C, 51.77; H, 5.18; N, 12.74.

### EXAMPLE 51

### Synthesis of (1S*, 2S*, 4S*, 5R*, 6S*)-2-Amino-4-[(pyridine-4-carbonyl)-amino]-bicyclo[3.1.0]hexane-2,6-dicarboxylic acid

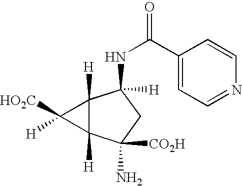

The title compound was prepared by the two step procedure in Example 1. The product of Preparation 1 (0.30 g, 0.84 mmol) was reacted with isonicotinoyl chloride hydrochloride (0.18 g, 1.01 mmol) in step (a). Purification of the desired intermediate was by PC-TLC (25% EtOAc/Hexane to 100% EtOAc) with a 80% yield. The intermediate, (1S*, 2S*, 4S*, 5R*, 6S*)-2-tert-Butoxycarbonylamino-4-[pyridine-4-carbonyl)-amino]-bicyclo[3.1.0]hexane-2,6-dicarboxylic acid diethyl ester (0.31 g, 0.68 mmol) was converted into the title compound by step (b) using isoelectric point crystalization from H₂O, pH 3, in a 37% yield (0.76 g, 0.25 mmol).

m.p.=>260° C. FDMS: M⁺−1=304. Anal. Calcd. For C₁₄H₁₅N₃O₅.0.6 HCl: C, 55.08; H, 4.95; N, 13.76. Found: C, 51.27; H, 4.80; N, 12.59.

### EXAMPLE 52

Synthesis of (1S*, 2S*, 4S*, 5R*, 6S*)-2-Amino-4-(2-trifluoromethyl-benzoylamino)-bicyclo[3.1.0]hexane-2,6-dicarboxylic acid

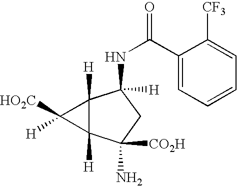

The title compound was prepared by the two step procedure in Example 1. The product of Preparation 1 (0.30 g, 0.84 mmol) was reacted with 2-(trifluoro-methyl)benzoyl chloride (0.21 g, 1.01 mmol) in step (a). Purification of the desired intermediate was by PC-TLC (10% EtOAc/Hexane to 50% EtOAc/Hexane) with a 99% yield (0.44 g, 0.83 mmol). The intermediate, (1S*, 2S*, 4S*, 5R*, 6S*)-2-tert-Butoxycarbonylamino-4-(2-trifluoromethyl-benzoylamino)-bicyclo[3.1.0]hexane-2,6-dicarboxylic acid diethyl ester (0.42 g, 0.80 mmol) was converted into the title compound by step (b) using isoelectric point crystalization from H₂O, pH 3, in a 63% yield (0.19 g, 0.50 mmol).

m.p.=243-244° C. FDMS: M⁺+1=373. Anal. Calcd. For C₁₆H₁₅N₂O₅F₃.0.5 H₂O: C, 50.40; H, 4.23; N, 7.35. Found: C, 50.04; H, 3.83; N, 7.29.

### EXAMPLE 53

### Synthesis of (1S*, 2S*, 4S*, 5R*, 6S*)-2-Amino-4-(3,5-difluoro-benzoylamino)-bicyclo[3.1.0]hexane-2,6-dicarboxylic acid

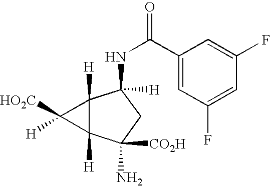

The title compound was prepared by the two step procedure in Example 1. The product of Preparation 1 (0.30 g, 0.84 mmol) was reacted with 3,5-difluorobenzoyl chloride (0.18 g, 1.01 mmol) in step (a). Purification of the desired intermediate was by PC-TLC (10% EtOAc/Hexane to 50% EtOAc/Hexane) with a 67% yield. The intermediate, (1S*, 2S*, 4S*, 5R*, 6S*)-2-tert-Butoxycarbonylamino-4-(3,5-difluoro-benzoylamino)-bicyclo[3.1.0]hexane-2,6-dicarboxylic acid diethyl ester (0.28 g, 0.56 mmol) was converted into the title compound by step (b) using isoelectric point crystalization from H₂O, pH 2-3 in a 83% yield (0.16 g, 0.47 mmol).

m.p.>260° C. FDMS: M⁺+1=341. Anal. Calcd. For C₁₅H₁₄N₂O₅F₂.0.1 H₂O: C, 52.67; H, 4.18; N, 8.19. Found: C, 52.28; H, 3.93; N, 8.03.

### EXAMPLE 54

### Synthesis of (1S*, 2S*, 4S*, 5R*, 6S*)-2-Amino-4-(2-methyl-benzoylamino)bicyclo[3.1.0]hexane-2,6-dicarboxylic acid

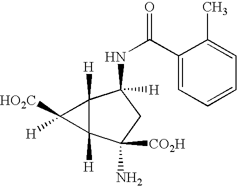

The title compound was prepared by the two step procedure in Example 1. The product of Preparation 1 (0.30 g, 0.84 mmol) was reacted with o-toluoyl chloride. (0.16 g, 1.01 mmol) in step (a). Purification of the desired intermediate was by PC-TLC (10% EtOAc/Hexane to 50% EtOAc/Hexane) with a 90% yield (0.36 g, 0.76 mmol). The. intermediate, (1S*, 2S*, 4S*, 5R*, 6S*)-2-tert-Butoxycarbonylamino-4-(2-methyl-benzoylamino)-bicyclo[3.1.0]hexane-2,6-dicarboxylic acid diethyl ester (0.34 g, 0.72 mmol) was converted into the title compound by step (b) using isoelectric point crystalization from H₂O, pH 3, in a 64% yield (0.15 g, 0.46 mmol).

m.p.=228-229° C. FDMS: M⁺+1=319. Anal. Calcd. For C₁₆H₁₈N₂O₅.0.5 H₂O: C, 58.71; H, 5.85; N, 8.56. Found: C, 58.49; H, 6.47; N. 8.47.

### EXAMPLE 55

### Synthesis of (1S*, 2S*, 4S*, 5R*, 6S*)-2-Amino-4-(4-methyl-benzoylamino)-bicyclo[3.1.0]hexane-2,6-dicarboxylic acid

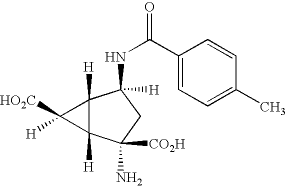

The title compound was prepared by the two step procedure in Example 1. The product of Preparation 1 (0.30 g, 0.84 mmol) was reacted with p-toluoyl chloride (0.16 g, 1.01 mmol) in step(a). Purification of the desired intermediate was by PC-TLC (10% EtOAc/Hexane to 30% EtOAc/Hexane) with a 95% yield (0.38 g, 0.80 mmol). The intermediate, (1S*, 2S*, 4S*, 5R*, 6S*)-2-tert-Butoxycarbonylamino-4-(4-methyl-benzoylamino)-bicyclo[3.1.0]hexane-2,6-dicarboxylic acid diethyl ester (0.37 g, 0.77 mmol) was converted into the title compound by step (b) using isoelectric point crystalization from H₂O, pH 3, in a 89% yield (0.22 g, 0.69 mmol).

m.p.=243-244° C. FDMS: M⁺+1=319. Anal. Calcd. For C₁₆H₁₈N₂O₅.0.2 H₂O: C, 59.69; H, 5.76; N, 8.70. Found: C, 59.32; H, 5.57; N, 8.50.

### EXAMPLE 56

### Synthesis of (1S*, 2S*, 4S*, 5R*, 6S*)-2-Amino-4-[(quinoxaline-2-carbonyl)-amino]-bicyclo[3.1.0]hexane-2,6-dicarboxylic acid

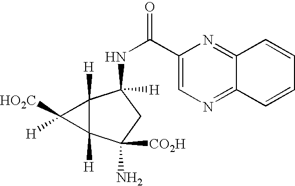

The title compound was prepared by the two step procedure in Example 1. The product of Preparation 1 (0.25 g, 0.70 mmol) was reacted with 2-quinoxaloyl chloride (0.16 g, 0.84 mmol) in step (a). Purification of the desired intermediate was by PC-TLC (25% EtOAc/Hexane to 100% EtOAc/Hexane) with a 84% yield (0.30 g, 0.59 mmol). (1S*, 2S*, 4S*, 5R*, 6S*)-2-tert-Butoxycarbonylamino-4-[(quinoxaline-2-carbonyl)-amino]-bicyclo[3.1.0]hexane-2,6-dicarboxylic acid diethyl ester (0.29 g, 0.57 mmol) was converted into the title compound by step (b) using isoelectric point crystalization from H₂O, pH 3, in a 83% yield (0.17 g, 0.47 mmol).

m.p.>260° C. FDMS: M⁺+1=357. Anal. Calcd. For C₁₇H₁₆N₄O₅.0.4 HCl: C, 55.05; H, 4.46; N, 15.11. Found: C, 54.67; H, 4.22; N, 14.88.

### EXAMPLE 57

### Synthesis of (1S*, 2S*, 4S*, 5R*, 6S*)-2-Amino-4-(3-carboxy-benzoylamino)-bicyclo[3.1.0]hexane-2,6-dicarboxylic acid

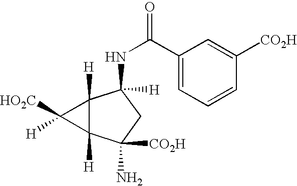

The title compound was prepared by the two step procedure in Example 2. The product of Preparation 1 (0.30 g, 0.84 mmol) was reacted with monomethyl isophthalate (0.17 g, 0.93 mmol) in step (a). Purification of the desired intermediate was by PC-TLC (10% EtOAc/Hexane to 30% EtOAc/Hexane) with a 78% yield (0.34 g, 0.66 mmol). The intermediate, (1S*, 2S*, 4S*, 5R*, 6S*)-2-tert-Butoxycarbonylamino-4-(3-carboxy-benzoylamino)-bicyclo[3.1.0]hexane-2,6-dicarboxylic acid diethyl ester (0.31 g, 0.60 mmol) was converted into the title compound by step (b) using isoelectric point crystalization from H₂O, pH 3, in a 89% yield (0.19 g, 0.53 mmol).

m.p.=249-250° C. FDMS: M⁺+1=349. Anal. Calcd. For C₁₆H₁₆N₂O₇.1.0 H₂O: C, 52.46; H, 4.95; N, 7.65. Found: C, 52.09; H, 4.59; N, 7.58.

### EXAMPLE 58

### Synthesis of (1S*, 2S*, 4S*, 5R*, 6S*)-2-Amino-4-(2-carboxy-benzoylamino)-bicyclo[3.1.0]hexane-2,6-dicarboxylic acid

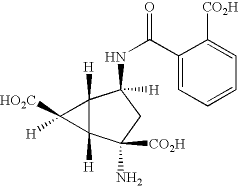

The title compound was prepared by the two step procedure in Example 2. The product of Preparation 1 (0.25 g, 0.70 mmol) was reacted with monomethyl phthalate (0.14 g, 0.77 mmol) in step (a). 1-[3-(Dimethylamino)propyl]-3-ethylcarboiimide hydrochloride was used (0.20 g, 1.05 mmol) in the place of 1,3-Dicyclohexylcarbodiimide. In the workup, extraction was EtOAc/H₂O (pH=1), followed by a straight H₂O wash of the organics. Purification of the desired intermediate was by PC-TLC (10% EtOAc/Hexane to 50% EtOAc/Hexane) with a 89% yield (0.32 g, 0.62 mmol). The intermediate, (1S*, 2S*, 4S*, 5R*, 6S*)-2-tert-Butoxycarbonylamino-4-(2-carboxy-benzoylamino)-bicyclo[3.1.0]hexane-2,6-dicarboxylic acid diethyl ester (0.31 g, 0.59 mmol) was converted into the title compound by step (b) using isoelectric point crystalization form H₂O, pH 1-2, in a 68% yield (0.14 g, 0.40 mmol).

m.p.>260° C. FDMS: M⁺+1=349. Anal. Calcd. For C₁₆H₁₆N₂O₇.0.5 HCl, 1.8 H₂O: C, 48.17; H, 5.08; N, 7.02. Found: C, 48.17; H, 4.80; N, 7.00.

### EXAMPLE 59

### Synthesis of (1S*, 2S*, 4S*, 5R*, 6S*)-2-Amino-4-(4-carboxy-benzoylamino)-bicyclo[3.1.0]hexane-2,6-dicarboxylic acid

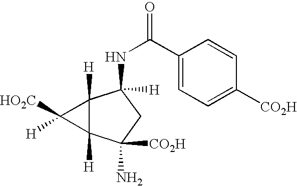

The title compound was prepared by the two step procedure in Example 2. The product of Preparation 1 (0.25 g, 0.70 mmol) was reacted with monomethyl terephthalate (0.14 g, 0.77 mmol) in step (a). 1-[3-(Dimethylamino)propyl]-3-ethylcarboiimide hydrochloride was used (0.20 g, 1.05 mmol) in the place of 1,3-Dicyclohexylcarbodiimide. In the workup, extraction was EtOAc/H₂O (pH=1), followed by a straight H₂O wash of the organics. Purification of the desired intermediate was by PC-TLC (10% EtOAc/Hexane to 30% EtOAc/Hexane) with a 88% yield (0.32 g, 0.62 mmol). The intermediate, (1S*, 2S*, 4S*, 5R*, 6S*)-2-tert-Butoxycarbonylamino-4-(4-carboxy-benzoylamino)-bicyclo[3.1.0]hexane-2,6-dicarboxylic acid diethyl ester (0.31 g, 0.59 mmol) was converted into the title compound by step (b) using isoelectric point crystalization from H₂O, pH 1-2, in a 85% yield (0.18 g, 0.50 mmol).

m.p.>260° C. FDMS: M⁺+1=349. Anal. Calcd. For C₁₆H₁₆N₂O₇.0.1 HCl, 0.7 H₂O: C, 52.71; H, 4.84; N, 7.68. Found: C, 52.52; H, 4.46; N, 7.52.

### EXAMPLE 60

### Synthesis of (1S*, 2S*, 4S*, 5R*, 6S*)-2-Amino-4-[(1H-imidazole-4-carbonyl)-amino]-bicyclo[3.1.0]hexane-2,6-dicarboxylic acid

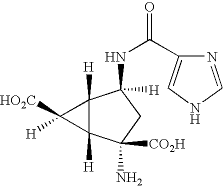

The title compound was prepared by the two step procedure in Example 2. The product of Preparation 1 (0.25 g, 0.70 mmol) was reacted with imidazol-4-carboxylic acid (0.0.9 g, 0.77 mmol) in step (a). 1-[3-(Dimethylamino)propyl]-3-ethylcarboiimide hydrochloride was used (0.20 g, 1.05 mmol) in the place of 1,3-Dicyclohexylcarbodiimide. In the workup, the organics were dried over anhydrous K₂CO₃. Purification of the desired intermediate was by PC-TLC (25% EtOAc/Hexane to 3% EtOH/EtOAc) with a 63% yield (0.20 g, 0.44 mmol). The intermediate, (1S*, 2S*, 4S*, 5R*, 6S*)-2-tert-Butoxycarbonylamino-4-[(1H-imidazole-4-carbonyl)-amino]-bicyclo[3.1.0]hexane-2,6-dicarboxylic acid diethyl ester (0.17 g, 0.38 mmol) was converted into the title compound by step (b) using isoelectric point crystalization from H₂O, pH 3, in a 47% yield (0.05 g, 0.18 mmol).

m.p.=258-259° C. FDMS: M⁺+1=295. Anal. Calcd. For C₁₂H₁₄N₄O₅.0.8 HCl: C, 44.56; H, 4.61; N, 17.32. Found: C, 44.54; H, 4.65; N, 16.96.

### EXAMPLE 61

### Synthesis of (1S*, 2S*, 4S*, 5R*, 6S*)-2-Amino-4-(3-phenyl-acroloylamino)-bicyclo[3.1.0]hexane-2,6-dicarboxylic acid

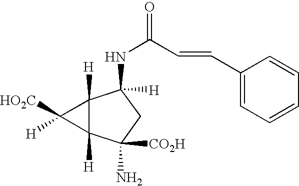

The title compound was prepared by the two step procedure in Example 2. The product of Preparation 1 (0.30 g, 0.84 mmol) was reacted with trans-cinnamic acid (0.14 g, 0.93 mmol) in step (a). 1-[3-(Dimethylamino)propyl]-3-ethylcarboiimide hydrochloride was used (0.24 g, 1.26 mmol) in the place of 1,3-Dicyclohexylcarbodiimide. In the workup, extraction was EtOAc/H₂O (pH=1), followed by a straight H₂O wash of the organics. Purification of the desired intermediate was by PC-TLC (10% EtOAc/Hexane to 30% EtOAc/Hexane) with a 63% yield (0.26 g, 0.53 mmol). The intermediate, (1S*, 2S*, 4S*, 5R*, 6S*)-2-tert-Butoxycarbonylamino-4-(3-phenyl-acroloylamino)-bicyclo[3.1.0]hexane-2,6-dicarboxylic acid diethyl ester (0.26 g, 0.53 mmol) was converted into the amine by step (b), the Boc group removal, 99.9% yield (0.21 g, 0.54 mmol). FDMS: M⁺+1=387. After hydrolysis of the esters, the title compound was isolated using isoelectric point crystalization from H₂O, pH 3, in a 79% yield (0.13 g, 0.39 mmol).

m.p.=225-226° C. FDMS: M⁺+1=331. Anal. Calcd. For C₁₇H₁₈N₂O₅. C, 61.81; H, 5.49; N, 8.48. Found: C, 61.52; H, 5.65; N, 8.12.

### EXAMPLE 62

### Synthesis of (1S*, 2S*, 4S*, 5R*, 6S*)-2-Amino-4-(3-phenyl-propynoylamino)-bicyclo[3.1.0]hexane-2,6-dicarboxylic acid

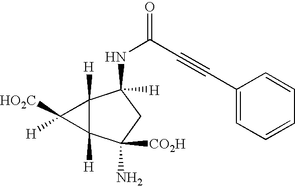

The title compound was prepared by the two step procedure in Example 2. The product of Preparation 1 (0.25 g, 0.70 mmol) was reacted with phenylpropiolic acid (0.11 g, 0.77 mmol) in step (a). 1-[3-(Dimethylamino)propyl]-3-ethylcarboiimide hydrochloride was used (0.20 g, 1.05 mmol) in the place of 1,3-Dicyclohexylcarbodiimide. In the workup, extraction was EtOAc/H₂O (pH=1), followed by a straight H₂O wash of the organics. Purification of the desired intermediate was by PC-TLC (10% EtOAc/Hexane to 30% EtOAc/Hexane) with a 48% yield (0.16 g, 0.34 mmol). The intermediate, (1S*, 2S*, 4S*, 5R*, 6S*)-2-tert-Butoxycarbonylamino-4-(3-phenyl-propynoylamino)-bicyclo[3.1.0]hexane-2,6-dicarboxylic acid diethyl ester (0.15 g, 0.31 mmol) was converted into the title compound by step (b) using isoelectric point crystalization from H₂O, pH 3, in a 64% yield (0.07 g, 0.20 mmol).

m.p.=222-223° C. FDMS: M⁺+1=329. Anal. Calcd. For C₁₇H₁₆N₂O₅.0.6 H₂O: C, 60.21; H, 5.11; N, 8.26. Found: C, 59.93; H, 5.05; N, 8.31.

### EXAMPLE 63

### Synthesis of (1S*, 2S*, 4S*, 5R*, 6S*)-2-Amino-4-benzylamino-bicyclo[3.1.0]hexane-2,6-dicarboxylic acid

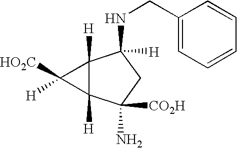

### A. (1S*, 2S*, 4S*, 5R*, 6S*)-4-Benzylamino-2-tert-butoxycarbonylamino-bicyclo[3.1.0]hexane-2,6-dicarboxylic acid diethyl ester.

Product from Preparation 1 (a) (0.30 g, 0.84 mmol) was dissolved in EtOH at ambient temperature under nitrogen. Benzyl bromide (0.15 g, 0.91 mmol) was added in one portion. The reaction mixture was stirred for 20 hours. The reaction mixture was concentrated under vacuum. Purification by PC-TLC (14% EtOAc/hexanes to 100% EtOAc) afforded the title product as the desired intermediate (0.14 g, 0.31 nmmol).

FDMS: M⁺+1=447.

### B. (1S*, 2S*, 4S*, 5R*, 6S*)-2-Amino-4-benzylamino-bicyclo[3.1.0]hexane-2,6-dicarboxylic acid

(1S*, 2S*, 4S*, 5R*, 6S*)-4-Benzylamino-2-tert-butoxycarbonylamino-bicyclo[3.1.0]hexane-2,6-dicarboxylic acid diethylester from step (a) plus additional intermediate (0.24 g, 0.54 mmol) in 30 mL of EtOAc was cooled to 0° C. under nitrogen. HCl (g) was added until the solution was saturated. The reaction was concentrated under vacuum. THF (8 mL) and deionized H₂O (1 mL) were added to the reaction concentrate. 1N NaOH (9.5 mL, 9.5 mmol) was added at ambient temperature, and the reaction was left stirring for 20 hour, pH 13-14. Using 1 N HCl, the reaction was taken to pH=7, concentrated under vacuum. The solids were reconstituted in H₂O. Purification was by anion-exchange chromatography (Bio-Rad® AG1-X8: elution with 3N AcOH). Compound diluted in CH₃OH, precipatate obtained upon addition of IPA. Crystals vacuum filtered, 80% yield (0.13 g, 0.43 mmol).

mp=244-245° C. FDMS: M⁺−1=289. Anal. Calcd. For C₁₅H₁₈N₂O₄.0.1 IPA, 0.7 H₂O: C, 59.48; H, 6.59; N, 9.07. Found: C, 59.15; H, 6.39; N, 8.71.

### EXAMPLE 64

### Synthesis of (1S*,2S*,4S*,5R*,6R*) 2-Amino-4-phenylcarbamoyloxy-bicyclo[3.1.0]hexane-2,6-dicarboxylic acid

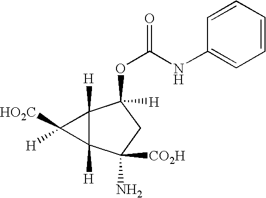

### A. (1S*,2S*,4S*,5R*,6R*) Diethyl 2-(N-t-butyloxycarbonyl-amino)-4-phenylcarbamoyloxy-bicyclo[3.1.0]hexane-2,6-dicarboxylic acid

A 0° C. solution of (1S*,2S*,4S*,5R*,6R*) diethyl 2-(N-t-butyloxycarbonylamino)-4-hydroxybicyclo[3.1.0]hexane-2,6-dicarboxylic acid (0.30 g, 0.84 mmol-synthesis previously described in U.S. Pat. No. 5,958,960 example 8a) in pyridine (10 mL) was treated in one portion with phenylisocyanate (0.12 g, 1.0 mmol) and allowed to warm to room temperature as it stirred overnight. The reaction mixture was partitioned between EtOAc and aqueous NaHSO₄ and the product extracted with EtOAc. All organic layers were combined, washed with brine, dried over MgSO₄, concentrated in vacuo and purified by SiO₂ chromatography (PC-TLC: 10% EtOAc/hexanes to 20% EtOAc/hexanes) to yield 0.30 g (0.63 mmol, 75%) of a white solid.

FDMS: M⁺+1=477. Anal. calcd. for C₂₄H₃₂N₂O₈.0.1 eq. CH₂Cl₂: C, 59.68; H, 6.69; N, 5.78. Found: C, 59.75; H, 6.59; N, 5.68.

### B. (1S*,2S*,4S*,5R*,6R*) 2-Amino-4-phenylcarbamoyloxy-bicyclo[3.1.0]hexane-2,6-dicarboxylic acid

A 0° C. solution of the product of example 21a (0.28 g, 0.59 mmol) in EtOAc (35 mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (15 mL total volume) and stirred at room temperature overnight. The reaction mixture was adjusted to pH=7 with 1N HCl and concentrated to dryness. The solids were reconstituted in water, adjusted to pH=12 with 1N NaOH and purified by anion exchange chromatography (Bio-Rad® AG1-X8 resin; acetate form converted to hydroxide form). The product was eluted from the column with 3N AcOH and concentrated to dryness in vacuo to afford crude product. Trituration in 2-propanol/H₂O (5:1) followed by drying under vacuum at 80° C. afforded 0.12 g (0.37 mmol, 63%) of the desired product as a white solid.

mp=dec>270° C. FDMS: M⁺+1=321. Anal. calcd. for C₁₅H₁₆N₂O₆.0.2 eq. H₂O: C, 55.62; H, 5.10; N, 8.65. Found: C, 55.51; H, 5.01; N, 8.39.

### EXAMPLE 65

### Synthesis of (1S*, 2S*, 4S*, 5R*, 6R*)-2-Amino-4-(2-chloro-phenylcarbamoyloxy)-bicyclo[3.1.0]hexane-2,6-dicarboxylic acid

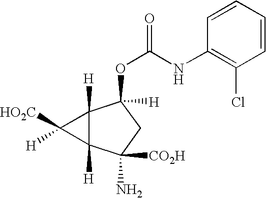

The title compound was prepared by the two step procedure Example Matt. (1S*, 2S*,4S*, 5R*, 6R*)-2-tert-Butoxycarbonylamino-4-hydroxy-bicyclo[3.1.0]hexane-2,6-dicarboxylic acid diethyl ester (0.30 g, 0.84 mmol) was reacted with o-chlorophenyl isocyanate (0.16 g, 1.01 mmol) in step (a). Purification of the desired intermediate was by PC-TLC (10% EtOAc/Hexane to 25% EtOAc/Hexane) with a 59% yield (0.25 g, 0.49 mmol). (1S*, 2S*,4S*, 5R*, 6R*)-2-tert-Butoxycarbonylamino-4-(2-chloro-phenylcarbamoyloxy)-bicyclo[3.1.0]hexane-2,6-dicarboxylic acid diethyl ester (0.25 g, 0.49 mmol) was converted into the title compound by step (b) using isoelectric point crystalization from H₂O, pH 3, in a 37% yield (0.06 g, 0.18 mmol).

m.p.=245-246° C. FDMS: M⁺+1=355. Anal. Calcd. For C₁₅H₁₅N₂O₆Cl.0.1 H₂O: C, 50.53; H, 4.30; N, 7.86. Found: C, 50.13; H, 3.93; N, 7.58.

### EXAMPLE 66

### Synthesis of (1S*, 2S*, 4S*, 5R*, 6R*)-2-Amino-4-(2-fluoro-phenylcarbamoyloxy)-bicyclo[3.1.0]hexane-2,6-dicarboxylic acid

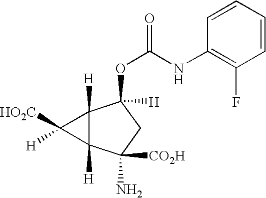

### A. (1S*, 2S*,4S*, 5R*, 6R*)-2-tert-Butoxycarbonylamino-4-(2-fluoro-phenylcarbamoyloxy)-bicyclo[3.1.0]hexane-2,6-dicarboxylic acid diethyl ester

To (1S*, 2S*,4S*, 5R*, 6R*)-2-tert-Butoxycarbonylamino-4-hydroxy-bicyclo[3.1.0]hexane-2,6-dicarboxylic acid diethyl ester (0.30 g, 0.84 mmol) in 10 mL of toluene, was added in one portion, 2-fluorophenyl isocyanate (0.26 g, 1.86 mmol). The reaction was set to reflux for 20 hours. Upon completion, indicated by TLC, the reaction was concentrated under vacuum. Purification of (1S*, 2S*,4S*, 5R*, 6R*)-2-tert-Butoxycarbonylamino-4-(2-fluoro-phenylcarbamoyloxy)-bicyclo[3.1.0]hexane-2,6-dicarboxylic acid diethyl ester was by PC-TLC (10%EtOAc/Hexane to 25% EtOAc/Hexane), with a 94% yield (0.39 g, 0.79 mmol).

FDMS: M⁺−1=493.

### B. (1S*, 2S*, 4S*, 5R*, 6R*)-2-Amino-4-(2-fluoro-phenylcarbamoyloxy)-bicyclo[3.1.0]hexane-2,6-dicarboxylic acid

A solution of the product from step (a) (0.36 g, 0.73 mmol), in EtOAc was chilled to 0° C. The reaction solution was saturated with HCl (g) and allowed to react until the Boc group was cleaved as indicated by TLC. The reaction was then concentrated under vacuum and rediluted in THF (12 mL). 1N NaOH (12 mL, 12 mmols) was added and the mixture was allowed to stir overnight (pH=13-14). The reaction mixture was titrated with 1N HCl to a pH=7, and the organics were removed under vacuum. The title compound was isolated by isoelectric point crystalization from H₂O, pH 3, in a 57% yield (0.14 g, 0.41 mmol) (A small amount of IPA was used to transfer the crystalsfrom the flask to drying funnel.).

m.p.=232-233° C. FDMS: M⁺+1=339. Anal. Calcd. For C₁₅H₁₅N₂O₆ F. 0.5 IPA, 0.6 H₂O: C, 52.27; H, 5.37; N, 7.39. Found: C, 52.21; H, 5.15; N, 7.39.

### EXAMPLE 67

### Synthesis of (1S*, 2S*, 4S*, 5R*, 6R*)-2-Amino-4-(3-fluoro-phenylcarbamoyloxy)-bicyclo[3.1.0]hexane-2,6-dicarboxylic acid

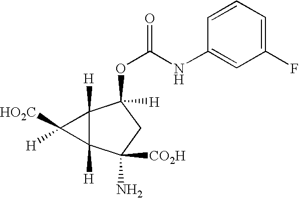

The title compound was prepared by the two step procedure in Example Bick. (1S*, 2S*,4S*, 5R*, 6R*)-2-tert-Butoxycarbonylamino-4-hydroxy-bicyclo[3.1.0]hexane-2,6-dicarboxylic acid diethyl ester (0.30 g, 0.84 mmol) was reacted with 3-fluorophenyl isocyanate (0.19 g, 1.35 mmol) in step (a). Purification of the desired intermediate was by PC-TLC (10% EtOAc/Hexane to 25% EtOAc/Hexane) with a 65% yield (0.27 g, 0.55 mmol). (1S*, 2S*,4S*, 5R*, 6R*)-2-tert-Butoxycarbonylamino-4-(3-fluoro-phenylcarbamoyloxy)-bicyclo[3.1.0]hexane-2,6-dicarboxylic acid diethyl ester (0.24 g, 0.49 mmol) was converted into the title compound by step (b) using isoelectric point crystalization from H₂O, pH 3 in a 54% yield (0.09 g, 0.27 mmol).

m.p.=239-240° C. FDMS: M⁺+1=339. Anal. Calcd. For C₁₅H₁₅N₂O₆ F.0.2 H₂O: C, 52.69; H, 4.54; N, 8.19. Found: C, 52.49; H, 4.78; N, 8.07.

### EXAMPLE 68

### Synthesis of (1S*, 2S*, 4S*, 5R*, 6R*)-2-Amino-4-(4-fluoro-phenylcarbamoyloxy)-bicyclo[3.1.0]hexane-2,6-dicarboxylic acid

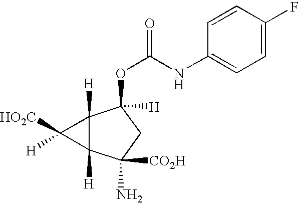

The title compound was prepared by the two step procedure in Example Bick. (1S*, 2S*,4S*, 5R*, 6R*)-2-tert-Butoxycarbonylamino-4-hydroxy-bicyclo[3.1.0]hexane-2,6-dicarboxylic acid diethyl ester (0.30 g, 0.84 mmol) was reacted with 4-fluorophenyl isocyanate (0.18 g, 1.11 mmol) in step (a). Purification of the desired intermediate was by PC-TLC (10% EtOAc/Hexane to 25% EtOAc/Hexane) with a 43% yield (0.28 g, 0.57 mmol). (1S*, 2S*,4S*, 5R*, 6R*)-2-tert-Butoxycarbonylamino-4-(4-fluoro-phenylcarbamoyloxy)-bicyclo[3.1.0]hexane-2,6-dicarboxylic acid diethyl ester (0.28 g, 0.57 mmol) was converted into the title compound by step (b) using isoelectric point crystalization from H₂O, pH 3 in a 41% yield (0.08 g, 0.23 mmol).

m.p.=259-260° C. FDMS: M⁺+1=339. Anal. Calcd. For C₁₅H₁₅N₂O₆ F. 0.1 NaCl: C, 52.35; H, 4.39; N, 8.14. Found: C, 51.98; H, 4.01; N, 7.96.

### EXAMPLE 69

### Synthesis of (1S*, 2S*, 4S*, 5R*, 6R*)-2-Amino-4-(3-chloro-phenylcarbamoyloxy)-bicyclo[3.1.0]hexane-2,6-dicarboxylic acid

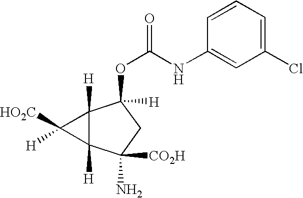

The title compound was prepared by the two step procedure in Example Bick. (1S*, 2S*,4S*, 5R*, 6R*)-2-tert-Butoxycarbonylamino-4-hydroxy-bicyclo[3.1.0]hexane-2,6-dicarboxylic acid diethyl ester (0.30 g, 0.84 mmol) was reacted with meta-chlorophenyl isocyanate (0.16 g, 1.01 mmol) in step (a). Purification of the desired intermediate was by PC-TLC (10% EtOAc/Hexane to 25% EtOAc/Hexane) with a 85% yield (0.37 g, 0.72 mmol). (1S*, 2S*,4S*, 5R*, 6R*)-2-tert-Butoxycarbonylamino-4-(3-chloro-phenylcarbamoyloxy)-bicyclo[3.1.0]hexane-2,6-dicarboxylic acid diethyl ester (0.37 g, 0.72 mmol) was converted into the title compound by step (b) using isoelectric point crystalization from H₂O, pH 3 in a 47% yield (0.12 g, 0.34 mmol).

m.p.=237-238° C. FDMS: M⁺+1=355. Anal. Calcd. For C₁₅H₁₅N₂O₆ Cl.0.5 H₂O: C, 49.53; H, 4.43; N, 7.70. Found: C, 49.53; H, 4.36; N, 7.59.

### EXAMPLE 70

### Synthesis of (1S*, 2S*, 4S*, 5R*, 6R*)-2-Amino-4-(4-chloro-phenylcarbamoyloxy)-bicyclo[3.1.0]hexane-2,6-dicarboxylic acid

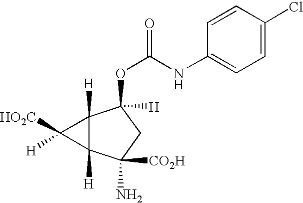

The title compound was prepared by the two step procedure in Example Bick. (1S*, 2S*,4S*, 5R*, 6R*)-2-tert-Butoxycarbonylamino-4-hydroxy-bicyclo[3.1.0]hexane-2,6-dicarboxylic acid diethyl ester (0.30 g, 0.84 mmol) was reacted with p-chlorophenyl isocyanate (0.16 g, 1.01 mmol) in step (a). Purification of the desired intermediate was by PC-TLC (10% EtOAc/Hexane to 25% EtOAc/Hexane) with a 74% yield (0.32 g, 0.62 mmol). (1S*, 2S*,4S*, 5R*, 6R*)-2-tert-Butoxycarbonylamino-4-(4-chloro-phenylcarbamoyloxy)-bicyclo[3.1.0]hexane-2,6-dicarboxylic acid diethyl ester (0.28 g, 0.54 mmol) was converted into the title compound by step (b) using isoelectric point crystalization from H₂O, pH 3, in a 73% yield (0.14 g, 0.39 mmol).

m.p.=258-259° C. FDMS: M⁺−1=353. Anal. Calcd. For C₁₅H₁₅N₂O₆Cl.0.2 H₂O: C, 50.27; H, 4.33; N, 7.82. Found: C, 50.12; H, 4.25; N, 7.72.

### EXAMPLE 71

### Synthesis of (1S*, 2S*, 4S*, 5R*, 6R*)-2-Amino-4-(naphthalen-1-ylcarbamoyloxy)-bicyclo[3.1.0]hexane-2,6-dicarboxylic acid

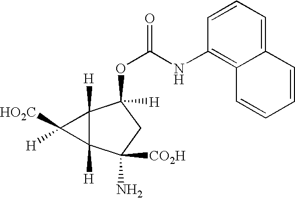

The title compound was prepared by the two step procedure in Example Bick. (1S*, 2S*,4S*, 5R*, 6R*)-2-tert-Butoxycarbonylamino-4-hydroxy-bicyclo[3.1.0]hexane-2,6-dicarboxylic acid diethyl ester (0.30 g, 0.84 mmol) was reacted with 1-naphthyl isocyanate (0.21 g, 1.26 mmol) in step (a). Purification of the desired intermediate was by PC-TLC (10% EtOAc/Hexane to 25% EtOAc/Hexane) with a 61% yield. By step (b), using LiOH for the ester hydrolysis, (1S*, 2S*,4S*, 5R*, 6R*)-2-tert-Butoxycarbonylamino-4-(naphthalen-1-ylcarbamoyloxy)-bicyclo[3.1.0]hexane-2,6-dicarboxylic acid diethyl ester (0.31 g, 0.59 mmol) was converted to the Boc-protected di-acid. Purification of the desired intermediate was by PC-TLC (0-2% AcOH in 50% EtOAc/Hexane) for a 61% yield (0.17 g, 0.36 mmol). The Boc group was cleaved, giving the title compound in a 63% yield (0.08 g, 0.23 mmol).

m.p.=238-239° C. FDMS: M⁺+1=371. Anal. Calcd. For C₁₉H₁₈N₂O₆.0.1 H₂O: C, 61.32; H, 4.93; N, 7.53. Found: C, 61.67; H, 4.86; N, 7.29.

### EXAMPLE 72

### Synthesis of (1S*, 2S*, 4S*, 5R*, 6R*)-2-Amino-4-(naphthalen-2-ylcarbamoyloxy)-bicyclo[3.1.0]hexane-2,6-dicarboxylic acid

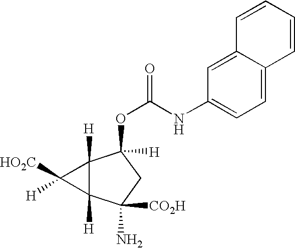

The title compound was prepared by the two step procedure in Example Bick. (1S*, 2S*,4S*, 5R*, 6R*)-2-tert-Butoxycarbonylamino-4-hydroxy-bicyclo[3.1.0)hexane-2,6-dicarboxylic acid diethyl ester (0.25 g, 0.70 mmol) was reacted with 2-naphthyl isocyanate (0.26 g, 1.51 mmol) in step (a). Purification of the desired intermediate was by PC-TLC (10% EtOAc/Hexane to 25% EtOAc/Hexane) with a 68% yield (0.25 g, 0.47 mmol). By step (b), using LiOH for the ester hydrolysis, (1S*, 2S*,4S*, 5R*, 6R*)-2-tert-Butoxycarbonylamino-4-(naphthalen-2-ylcarbamoyloxy)-bicyclo[3.1.0]hexane-2,6-dicarboxylic acid diethyl ester (0.28 g, 0.53 mmol) was converted to the Boc-protected di-acid followed by cleavage of the Boc group. The title compound was isolated using isoelectric point crystalization from H₂O, pH 3, in a 20% yield (0.04 g, 0.11 mmol)

m.p.=238-239° C. FDMS: M⁺−1=369. Anal. Calcd. For C₁₉H₁₈N₂O₆.0.8 HCl: C, 57.12; H, 4.74; N, 7.01. Found: C, 57.01; H, 4.82; N, 6.85.

### EXAMPLE 73

### Synthesis of (1S*,2S*,4S*,5R*,6R*) 2-Amino-4-(2,4-dichlorophenoxy)bicyclo[3.1.0]hexane-2,6-dicarboxylic acid

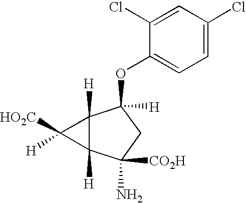

### A. (1S*,2S*,4S*,5R*,6R*) Diethyl 2-(N-t-butyloxycarbonyl-amino)-4-[(2,4-dichlorophenoxy)bicyclo[3.1.0]hexane-2,6-dicarboxylic acid

A 0° C. solution of (1S*,2S*,4S*,5R*,6R*) diethyl 2-(N-t-butyloxycarbonylamino)-4-hydroxybicyclo[3.1.0]hexane-2,6-dicarboxylic acid (1.0 g, 2.8 mmol-synthesis previously described in U.S. Pat. No. 5,958,960 example 8a) in N,N-dimethylformamide (10 mL) was treated in one portion with sodium hydride (0.13 g, 3.4 mmol) and stirred vigorously for 15 minutes. The resulting reaction mixture was treated in one portion with 1,3-dichloro-4-fluorobenzene (0.92 g, 5.6 mmol) and allowed to warm to room temperature as it stirred overnight. The reaction mixture was partitioned between EtOAc and water and the product extracted with EtOAc. All organic layers were combined, washed with brine, dried over MgSO₄, concentrated in vacuo and purified by SiO₂ chromatography (PC-TLC: 10% EtOAc/hexanes to 50% EtOAc/hexanes) to yield 0.63 g (1.3 mmol, 45%) of a white foam.

FDMS: M⁺=502, 504. Anal. calcd. for C₂₃H₂₉Cl₂NO₇.0.1 eq. CH₂Cl₂: C, 54.31; H, 5.76; N, 2.74. Found: C, 54.56; H, 5.75; N, 2.93.

### B. (1S*,2S*,4S*,5R*,6R*)2-Amino)-4-(2,4-dichlorophenoxy)-bicyclo[3.1.0]hexane-2,6-dicarboxylic acid

A 0° C. solution of the product of example 20a (0.62 g, 1.23 mmol) in EtOAc (35 mL) was purged with anhydrous HCl gas until the solution was saturated. The resulting reaction mixture was stirred at 0° C. for 4 hours and concentrated to dryness in vacuo to afford the HCl salt. The resulting solids were reconstituted in a 1:1 mixture of 1N NaOH:THF (20 mL total volume) and stirred at room temperature overnight. The reaction mixture was adjusted to pH=7 with 1N HCl and solids removed and filtrate concentrated to dryness in vacuo. The solids were reconstituted in minimal amount of water and the product isolated by isoelectric precipitation at pH=2-3. The product was dried under vacuum at 80° C. to afford 0.38 g (1.1 mmol, 89%) of the desired product as a white solid.

mp=>250° C. FDMS: M⁺=346, 348. Anal. calcd. for C₁₄H₁₃Cl₂NO₅.0.3 eq. H₂O: C, 47.83; H, 3.90; N, 3.98. Found: C, 47.55; H, 3.61; N, 3.97.

### EXAMPLE 74

### Synthesis of (1S*,2S*,4R*,5R*,6S*)2-Amino-4-phenyl-carbamoylmethylbicyclo[3.1.0]hexane-2,6-dicarboxylic acid

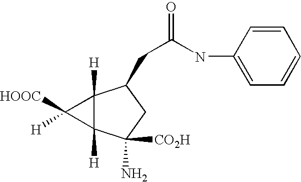

### A. 4(Z/E)-(1S*,2S*,5R*,6S*) Diethyl-2-(tert-butyloxy carbonylamino)-4-allyloxycarbonylmethylenebicyclo [3.1.0]hexane-2,6-dicarboxylate

To a 0° C. solution of allyl diethylphosphonoacetate (16.8 mmmol, 3.8 g.) in anhydrous THF, was added 1M NaHMDS (16.8 mmol) by dropwise addition. The resulting reaction mixture was stirred at 0° C. for 1 h, after which time a solution of (1S*,2S*,5R*,6R*) diethyl 2-N-tert-butyloxycarbonylamino-4-oxobicyclo[3.1.0]hexane-2,6-dicarboxylate (previously disclosed U.S. Pat. No. 5,958,960 example 5a-11.25 mmol, 3.9 g) in THF was added dropwise. The reaction mixture was allowed to room temperature as it stirred overnight. The reaction was quenched with saturated aqueous NH₄Cl solution and the product extracted with ethyl acetate. The combined organic extracts were dried over sodium sulfate, concentrated to dryness in vacuo, and purified by SiO₂ chromatography. (50% EtOAc/hexanes) to afford 4.8 g (10.9 mmol, 97%) of the title compound as a (1:1.5) mixture of diastereoisomers as colorless oil.

¹H-NMR (CDCl₃) δ: 5.91-5.77 (m, 4H), 5.45-5.14 (m, 6H), 4.60-4.55 (m, 4H), 4.25-4.00 (m, 8H), 3.89 (d, 1H, J=19.8 Hz), 3.64 (dd, 1H, J=2.7 and 5.9 Hz), 3.25 (d, 1H J=17.4 Hz), 2.79 (m, 1H), 2.63-2.57 (m, 2H), 2.24-2.08 (m, 2H), 2.07-2.02 (m, 2H), 1.4 (s, 9 H), 1.39 (s, 9H), 1.22 (dt, 12H) ppm. ¹³C-NMR (CDCl₃) δ: 172.2, 172.0, 170.3, 170.1, 165.6, 165.3, 160.1, 159.9, 132.2, 117.7, 113.5, 113.0, 65.3, 64.5, 63.5, 61.8, 61.7, 61.1, 60.2, 41.3, 38.5, 36.6, 35.8, 35.3, 31.5, 28.0, 25.4, 25.1, 14.0, 13.9 ppm. MS(ES) for C₂₂H₃₁NO₈ [M+H]⁺: 438.18; [M−H]⁻: 436.4.

### B. 4(Z/E)-(1S*,2S*,5R*,6S*) Diethyl-2-(tert-butyloxycarbonylamino)-4-carboxymethylene bicyclo [3.1.0) hexane-2,6-dicarboxylate

Method B1) An EtOH/H₂O (9:1) solution of the mixture of compounds from Example 74A (4.3 mmol, 1.9 g) was treated with Wilkinson's catalyst (Chlorotris triphenylphosphine)rhodium (I) (0.4 mmol, 0.370 g) and the resulting reaction mixture warmed at reflux for 5 hours. Upon cooling to room temperature, the mixture reaction was filtered through a pad of celite to remove the catalyst. The solvent was removed in vacuo and the crude reaction was passed through a short pad of SiO₂ to afford the title compound as a (1:1.5) mixture of isomers (1.28 g, 3.2 mmol, 73%). Method B2) Tetrakistriphenylphosphine Palladium (0) (0.150 g, 0.13 mmol) and pyrrolidine (0.48 g, 6.7 mmol) were sequentially added to a solution of the mixture of isomers from Example 74A (4.5 mmol, 2 g) in CH₂Cl₂ and the resulting reaction mixture warmed at reflux over night. The reaction was allowed to cool to room temperature and washed with NaHSO₄ and brine. The organic layer was concentrated to dryness, the residue diluted in MeOH, and the remaining insolubles removed via vacuum filtration. The filtrate was concentrated in vacuo and purified by filtration through a short pad of SiO₂ to afford a 1:1.5 mixture of the free carboxylic acids (0.9 g, 2.2 mmol, 53%).

¹H-NMR (CDCl₃) δ: 5.92 (s, 1H), 5.78 (s, 1H), 5.33 (brs, 2H), 4.25-4.09 (m, 8H), 3.95 d, 1H, J=19.0 Hz), 3.64 (dd, 1H, J=2.6 and 6.1 Hz), 2.77 (dd, 1H, J=3.2 and 5.9 Hz), 2.68-2.64 (m, 1H), 2.27-2.00 ((m, 4H), 1.43 (s, 18 H), 1.31-1.21 (m, 12H) ppm.

### C. 4(Z/E), (1S*,2S*,5R*,6S*) Diethyl 2-tert-butyloxy-carbonylamino-4-phenyl carbamoyl methylenebicyclo[3.1.0]-hexane-2,6-dicarboxylate

A room temperature solution of the product of Example 74B (0.5 g, 1.25 mmol) in CH₂Cl₂ was treated consecutively with oxalyl chloride (0.239 g, 1.89 mmol) and a catalytic amount of DMF. After the reaction mixture was stirred for one hour, the solvent was removed in vacuo. The crude product was reconstituted in CH₂Cl₂ and then a solution of aniline (0.232 g, 2.5 mmol) was added dropwise. The reaction mixture was allowed to stir at room temperature until the reaction was judged complete by TLC. The resulting mixture was concentrated to dryness and the crude product purified by filtration through a short pad of SiO₂ to afford the title compounds (0.422 g, 0.89 mmol) 71% as a mixture of diastereoisomers which were used in the next step without further separation.

MS(ES) for C₂₅H₃₂N₂O₇ [M+H]⁺: 473.2

### D. (1S*,2S*,4R*,5R*,6S*) Diethyl 2-tert-butyloxycarbonyl-amino-4-phenylcarbamoylmethylbicyclo[3.1.0]hexane-2,6-dicarboxylate

A room temperature solution of the product of Example 74C (0.422 g, 0.89 mmol) and 10% Pd/C (0.021 g, 5 wt %) in 95% EtOH was vigorous stirred under an hydrogen atmosphere (1 atm) overnight. The catalyst was removed via vacuum filtration through celite and the filtrate concentrated in vacuo to afford the title compound (0.34 g, 0.45 mmol, 80%).

¹H-NMR (CDCl₃) δ: 8.03 (s, 1H), 7.54 (d, 2H, J=7.8 Hz), 7.30 (t, 2H), 7.07 (t, 1H, J=7.5 Hz), 5.22 (s, 1H), 4.25-4.20 (m, 2H), 4.12 (q, 2H, J=7.2 Hz), 2.98-2.86 (m, 1H), 2.67 (d, 1H, J=15 Hz), 2.54 (d, 2H, J=8.3 Hz), 2.36 (dd, 1H, J=2.7 and 5.9 Hz), 1.88 (dd, 1H, J=3.5 and 6.0 Hz), 1.77 (t, 1H, J=2.9 Hz), 1.52-1.20 (m, 15 H) ppm.

MS(ES) for C₂₅H₃₄N₂O₇ [M+H]⁺: 475.07; [M−H]⁻: 473.23.

### E. (1S*,2S*,4R*,5R*,6S*) Diethyl 2-amino-4-phenylcarbamoylmethylbicyclo[3.1.0] hexane-2,6-dicarboxylate

The product of example 74D (0.297 g, 0.62 mmol) was treated with a saturated solution of EtOAc/HCl and stirred at room temperature over night. The reaction mixture was adjusted to pH=7-8 with NaHCO₃, and the product extracted with EtOAc. The crude product was purified by flash chromatography to afford the title compound (0.152 g, 0.4 mmol, 65%).

¹H-NMR (CDCl₃) δ: 8.18(s, 1H, NHCO), 7.55 (d, 2H, J=12 Hz), 7.24-7.12 (m, 2H), 6.89-6.78 (m, 1H), 4.27 (q, 2H, J=7.1 Hz), 4.11 (q, 2H, J=7.0 Hz), 2.84-2.75 (m, 1H), 2.52-2.25 (m, 4H), 1.86-1.84 (m, 3H), 1.77 (t, 1H, J=2.9 Hz), 1.40-1.21 (dt, 6H) ppm. MS(ES) for C₂₀H₂₆N₂O₅ [M+H]⁺: 375.04; [M−H]⁻: 373.15

### F. (1S*,2S*,4R*,5R*,6S*)-2-amino-4-phenycarbamoylmethyl-bicyclo[3.1.0]hexane-2,6-dicarboxylic acid

A solution of the product of Example 74E (0.083 g, 0.22 mmol) in THF was treated with 1N NaOH (0.55 mmol) and stirred at room temperature over night. The solvent was removed in vacuo and the crude product reconstituted in distilled water and acidified with 1N HCl. The product was isolated by isoelectric precipitation at pH=3-5. The product was collected via vacuum filtration, washed with water and air dried to afford the title compound (0.035 g, 0.11 mmol, 51%).

¹H-NMR (D₂O+KOD) δ: 7.37-7.34 (m, 4H), 7.22-7.17 (m, 1H), 2.64-2.37 (m, 3H), 2.03 (dd, 1H, J=3.3 and 6.3 Hz), 1.88 (d, 1H, J=14.9 Hz), 1.67 (dd, 1H, J=2.5 and 5.8 Hz), 1.46-1.33 (m, 2H) ppm. ¹³C-NMR (D₂O+KOD) δ: 183.7, 176.2, 138.7, 131.0, 127.4, 124.0, 67.9, 43.6, 42.7, 39.7, 37.3, 33.6, 26.8 ppm. MS(ES) for C₁₆H₁₈N₂O₅5 [M−H]⁻: 317.22 Pf>190° dec.

### EXAMPLE 75

### Synthesis of (1S*,2S*,4R*,5R*,6S*) 2-Amino-4-(o-methoxy)-phenylcarbamoylmethyl bicyclo[3.1.0]hexane-2,6-dicarboxylic acid

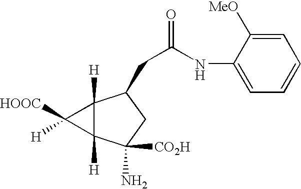

### A. 4(Z/E), (1S*,2S*,5R*,6S*) Diethyl 2-tert-butyloxy-carbonylamino-4-(o-methoxy)phenylcarbamoylmethylene bicyclo[3.1.0]hexane-2,6-dicarboxylate

A room temperature solution of the product of Example 74B (0.5 g, 1.25 mmol) in CH₂Cl₂ was treated consecutively with oxalyl chloride (0.239 g, 1.89 mmol) and a catalytic amount of DMF. After the reaction mixture was stirred for one hour, the solvent was removed in vacuo. The crude product was reconstituted in CH₂Cl₂ and then a solution of o-anisidine (0.307 g, 2.5 mmol) was added dropwise. The reaction mixture was allowed to stir at room temperature until the reaction was judged complete by TLC. The resulting mixture was concentrated to dryness and the crude product purified by filtration through a short pad of SiO₂ to afford the title compounds (0.260 g, 0.51 mmol) 40% as a mixture of diastereoisomers which were used in the next step without further separation.

MS(ES) for C₂₆H₃₄N₂O₈ [M+H]⁺: 502.11

### B. (1S*,2S*,4R*,5R*,6S*) Diethyl 2-tert-butyloxycarbonyl-amino-4-(o-methoxy) phenylcarbamoylmethylbicyclo[3.1.0]-hexane-2,6-dicarboxylate

A room temperature solution of the product of Example 75A (0.260 g, 0.51 mmol) and 10% Pd/C (0.021 g, 5 wt %) in 95% EtOH was vigorous stirred under an hydrogen atmosphere (1 atm) over night. The catalyst was removed via vacuum filtration through celite and the filtrate concentrated in vacuo to afford the title compound (0.150 g, 0.29 mmol, 60%).

C₂₆H₃₆N₂O₈ [M+H]⁺: 505.09. [M−H]⁻: 503.28

### C. (1S*,2S*,4R*,5R*,6S*) Diethyl 2-Amino-4-(o-methoxy) phenylcarbamoylmethyl bicyclo[3.1.0]hexane-2,6-dicarboxylate

The title compound was prepared as example 74E to give the product in 57% yield.

¹H NMR (CDCl₃) δ: 8.32 (d, 1H, J=7.6 Hz), 7.97 (s, 1H), 7.02-6.82 (m, 3H), 4.25 (q, 2H, J=7.1 Hz ), 4.10 (dq, 2H, J=1.3 and 7.2 Hz), 3.85 (s, 3H), 2.87-2.82 (m, 1H), 2.51-2.46 (m, 2H), 2.32 (dd, 1H, J=2.9 and 6.0 Hz), 2.21 (d 1H, J=14.8 Hz), 1.91 (dd, 1H, J=3.0 and 6.1.Hz), 1.76-1.72 (m, 3H), 1.44 (dd, 1H, J=8.3 and 14.8 Hz), 1.37-1.20 (m, 6H) ppm.

MS(ES) for C₂₁H₂₈N₂O₆ [M+H]⁺: 405.04; [M−H]⁻: 403.16

### D. (1S*,2S*,4R*,5R*,6S*) 2-Amino-4-(o-methoxy)phenyl carbamoylmethyl bicyclo[3.1.0]hexane-2,6-dicarboxylic acid

A solution of the product of Example 75C (0.068 g, 0.16 mmol) in THF was treated with 1N NaOH (0.33 mmol) and stirred at room temperature over night. The solvent was removed in vacuo and the crude product reconstituted in distilled water and acidified with 1N HCl. The product was isolated by isoelectric precipitation at pH=3-5. The product was collected via vacuum filtration, washed with water and air dried to afford the title compound (0.030 g, 0.08mmol, 52%).

¹H-NMR (MeOD+Pyr) δ: 7.36 (dd, 1H, J=1.4 and 7.7 Hz); 7.20 (m, 1H); 7.03-6.88 (m, 2H); 3.76 (s, 3H); 2.57-2.35 (m, 3H), 2.01 (dd, 1H, J=2.8 and 5.8 Hz), 1.89 (d, 1H, J=14.4 Hz); 1.72 (dd, 1H, J=2.9 and 6.0 Hz); 1.47-1.36 (m, 2H) ppm. ¹³C-NMR (D₂O+KOD) δ: 183.4, 181.9, 174.8, 152.3, 127.4, 125.8, 121.6, 112.3, 66.0, 55.9, 50.4, 41.1, 38.0, 35.6, 31.7, 25.1 ppm MS(ES) for C₁₇H₂₀N₂O ₆ [M+H]⁺: 348.99 [M−H]⁻: 347.08

### EXAMPLE 76

### Synthesis of (1S*,2S*,4R*,5R*,6S*) 2-Amino-4-(o-methyl) phenylcarbamoylmethyl bicyclo[3.1.0)hexane-2,6-dicarboxylic acid

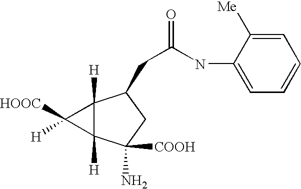

### A. 4(Z/E), (1S*,2S*,5R*,6S*) Diethyl 2-tert-butyloxy carbonylamino-4-(o-methyl)phenylcarbamoyl methylene bicyclo[3.1.0]hexane-2,6-dicarboxylate

A room temperature solution of the product of Example 74B (0.5 g, 1.25 mmol) in CH₂Cl₂ was treated consecutively with oxalyl chloride (0.239 g, 1.89 mmol) and a catalytic amount of DMF. After the reaction mixture was stirred for one hour, the solvent was removed in vacuo. The crude product was reconstituted in CH₂Cl₂ and then a solution of o-toluididne (0.268 g, 2.5 mmol) was added dropwise. The reaction mixture was allowed to stir at room temperature until the reaction was judged complete by TLC. The resulting mixture was concentrated to dryness and the crude product purified by filtration through a short pad of SiO₂ to afford the title compounds (0.370 g, 0.79 mmol) 60% as a mixture of diastereoisomers which were used in the next step without further separation.

### B. (1S*,2S*,4R*,5R*,6S*) Diethyl 2-tert-butyloxy carbonylamino-4-(o-methyl)phenylcarbamoylmethyl bicyclo[3.1.0]hexane-2,6-dicarboxylate

A room temperature solution of the product of Example 76A (0.370 g, 0.76 mmol) and 10% Pd/C (0.20 g, 5 wt %) in 95% EtOH was vigorous stirred under an hydrogen atmosphere (1 atm) over night. The catalyst was removed via vacuum filtration through celite and the filtrate concentrated in vacuo to afford the title compound (0.224 g, 0.45 mmol, 62%).

### C. (1S*,2S*,4R*,5R*,6S*) Diethyl 2-Amino-4-(o-methyl)phenylcarbamoylmethylbicyclo[3.1.0]hexane-2,6-dicarboxylate

The title compound was prepared using the same procedure as in Example 74E to give the product.

¹H NMR (CDCl₃) δ: 7.83 (d, 1H, J=7.7Hz), 7.71 (s, 1H), 7.20-6.99 (m, 3H), 4.26-4.04 (dq, 4H), 2.87-2.75 (m, 1H), 2.50-2.21 (m, 8H), 1.86 (dd, 1H, J=3.0 and 6.1 Hz), 1.76 (br s, 3H), 1.41-1.18 (m, 6H) ppm.

MS(ES) for C₂₁H₂₈N₂O₅ [M+H]⁺: 389.06; [M−H]⁻: 387.16

### D. (1S*,2S*,4R*,5R*,6S*) 2-Amino-4-(o-methyl)phenyl carbamoylmethyl bicyclo[3.1.0] hexane-2,6-dicarboxylic acid

A solution of the product of Example 76C (0.090 g, 0.23 mmol) in THF was treated with 1N NaOH (0.46 mmol) and stirred at room temperature over night. The solvent was removed in vacuo and the crude product reconstituted in distilled water and acidified with 1N HCl. The product was isolated by isoelectric precipitation at pH=3-5. The product was collected via vacuum filtration, washed with water and air dried to afford the title compound (0.030 g, 0.09 mmol, 39 %). ¹H-NMR (MeOD+Pyr) δ: 7.26-7.15 (m, 4H); 2.62-2.42 (m, 3H), 2.15 (s, 3H); 2.05 (dd, 1H, J=3.1 and 6.1 Hz), 1.94 (d, 1H, J=14.5 Hz); 1.72 (dd, 1H, J=2.6 and 6.0 Hz); 1.51-1.40 (m, 2H) ppm. ¹³C-NMR (D₂O+KOD) δ: 183.4, 181.9, 174.9, 134.7, 134.5, 130.7, 127.5, 126.9, 126.5, 66.1, 41.1, 37.9, 35.5, 31.9, 25.0, 17.0 ppm

MS(ES) for C₁₇H₂₀N₂O₅ [M+H]⁺: 332.99 [M−H]⁻: 331.09

### EXAMPLE 77

### Synthesis of (1S*,2S*,4R*,5R*,6S*) 2-Amino-4-(o-trifluoro methyl)phenylcarbamoylmethyl bicyclo[3.1.0]hexane-2,6-dicarboxylic acid

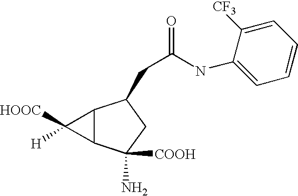

### A. 4(Z/E),(1S*,2S*,5R*,6S*) Diethyl 2-tert-butyloxy-carbonylamino-4-(o-trifluoromethyl)phenyl carbamoyl methylene bicyclo[3.1.0]hexane-2,6-dicarboxylate

A room temperature solution of the product of Example 74B (0.5 g, 1.25 mmol) in CH₂Cl₂ was treated consecutively with oxalyl chloride (0.239 g, 1.89 mmol) and a catalytic amount of DMF. After the reaction mixture was stirred for one hour, the solvent was removed in vacuo. The crude product was reconstituted in CH₂Cl₂ and then a solution of o-trifluoro methyl aniline (0.402 g, 2.5 mmol) was added dropwise. The reaction mixture was allowed to stir at room temperature until the reaction was judged complete by TLC. The resulting mixture was concentrated to dryness and the crude product purified by filtration through a short pad of SiO₂ to afford the title compounds (0.480 g, 0.88 mmol) 79 W as a mixture of diastereoisomers which were used in the next step without further separation. MS(ES) for C₂₆H₃₁F₃N₂O₇ [M+H]⁺: 541.21 [M−H]⁻: 539.24.

### B. (1S*,2S*,4R*,5R*,6S*) Diethyl 2-tert-butyloxy-carbonylamino-4-(o-trifluoromethyl)phenyl carbamoylmethyl-bicyclo[3.1.0]hexane-2,6-dicarboxylate

A room temperature solution of the product of Example 77A (0.370 g, 0.76 mmol) and 10% Pd/C (0.20 g, 5 wt %) in 95% EtOH was vigorous stirred under an hydrogen atmosphere (1 atm) over night. The catalyst was removed via vacuum filtration through celite and the filtrate concentrated in vacuo to afford the title compound

MS(ES) for C₂₆H₃₃F₃N₂O₇ [M+H]⁺: 543.09 [M−H]⁻: 541.24

### C. (1S*,2S*,4R*,5R*,6S*) Diethyl 2-Amino-4-(o-trifluoromethyl)phenylcarbamoyl methylbicyclo[3.1.0]hexane-2,6-dicarboxylate

The title compound was prepared using the same procedure as in Example 74E to give the product.

### D. (1S*,2S*,4R*,5R*,6S*) 2-Amino-4-(o-trifluoro methyl)phenylcarbamoylmethylbicyclo[3.1.0]hexane-2,6-dicarboxylic acid

A solution of the product of Example 77C (0.090 g, 0.20 mmol) in THF was treated with 1N NaOH (0.40 mmol) and stirred at room temperature over night. The solvent was removed in vacuo and the crude product reconstituted in distilled water and acidified with 1N HCl. The product was isolated by isoelectric precipitation at pH=3-5. The product was collected via vacuum filtration, washed with water and air dried to afford the title compound (0.045 g, 0.11 mmol, 57

¹H-NMR (MeOD+Pyr) □: 7.65 (d, 1H, J=7.6 Hz); 7.52 (t, 1H, J=7.1 Hz); 7.27 (t, 1H, J=7.7 Hz); 7.14 (d, 1H, J=7.8 Hz); 2.64-2.34 (m, 3H), 2.04 (dd, 1H, J=2.8 and 6.0 Hz), 1.94 (d, 1H, J=14.4 Hz); 1.76 (dd, 1H, J=2.9 and 6.1 Hz); 1.50-1.39 (m, 2H) ppm. ¹³C-NMR (D₂O+KOD) δ: 183.5, 182.1, 174.9, 132.8, 128.8, 126.3, 125.3, 66.1, 42.2, 41.8, 38.4, 35.6, 32.2, 25.2 ppm

### EXAMPLE 78

### Synthesis of (1S*,2S*,4R*,5R*,6S*) 2-Amino-4-(o-chloro)phenylcarbamoylmethyl bicyclo[3.1.0]hexane-2,6-dicarboxylic acid

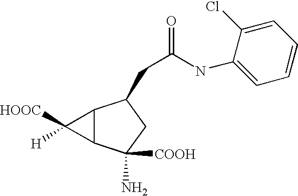

### A. 4(Z/E), (1S*,2S*,5R*,6S*) Diethyl 2-tert-butyloxy-carbonylamino-4-(o-chloro) phenylcarbamoylmethylene bicyclo[3.1.0]hexane-2,6-dicarboxylate

A room temperature solution of the product of Example 74B (0.5 g, 1.25 mmol) in CH₂Cl₂ was treated consecutively with oxalyl chloride (0.239 g, 1.89 mmol) and a catalytic amount of DMF. After the reaction mixture was stirred for one hour, the solvent was removed in vacuo. The crude product was reconstituted in CH₂Cl₂ and then a solution of o-chloroaniline (0.318 g, 2.5 mmol) was added dropwise. The reaction mixture was allowed to stir at room temperature until the reaction was judged complete by TLC. The resulting mixture was concentrated to dryness and the crude product purified by filtration through a short pad of SiO₂ to afford the title compounds (0.283 g, 0.55 mmol) 44% as a mixture of diastereoisomers which were used in the next step without further separation.

### B. (1S*,2S*,4R*,5R*,6S*) Diethyl 2-tert-butyloxy carbonylamino-4-(o-chloro)phenylcarbamoyl methylbicyclo[3.1.0]hexane-2,6-dicarboxylate

A room temperature solution of the product of Example 78A (0.283 g, 0.55 mmol) and 10% Pd/C (0.014 g, 5 wt %) in 95% EtOH was vigorous stirred under an hydrogen atmosphere (1 atm) over night. The catalyst was removed via vacuum filtration through celite and the filtrate concentrated in vacuo to afford the title compound (0.210 g, 0.41 mmol, 74%). for C₂₅H₃₃ClN₂O₇ [M+H]⁺—Cl: 475.08 [M−H]⁻—Cl: 473.22

### C. (1S*,2S*,4R*,5R*,6S*) Diethyl 2-Amino-4-(o-chloro) phenylcarbamoyl methylbicyclo[3.1.0]hexane-2,6-dicarboxylate

The title compound was prepared as in Example 74E to give the product in 67% yield

¹H NMR (CDCl₃) δ: 8.07 (s,1H), 7.53 (d, 2H, J=7.9 Hz), 7.33-7.25 (m, 1H), 7.07 (t, 1H, J=7.3 Hz), 4.26 (q, 2H, J=7.1 Hz ), 4.19-4.06 (m, 2H), 2.87-2.75 (m, 1H), 2.45-2.24 (m, 5H), 1.87-1.75 (m, 4H), 1.34-1.20 (m, 6H) ppm. MS(ES) for C₂₀H₂₅ClN₂O₆ [M+H]⁺/—Cl: 375.04; [M−H]⁻/—Cl: 373.11

### D. (1S*,2S*,4R*,5R*,6S*) 2-Amino-4-(o-chloro)phenyl carbamoylmethyl bicyclo[3.1.0]hexane-2,6-dicarboxylic acid

A solution of the product of Example 78C (0.150 g, 0.36 mmol) in THF was treated with 1N NaOH (0.73 mmol) and stirred at room temperature over night. The solvent was removed in vacuo and the crude product reconstituted in distilled water and acidified with 1N HCl. The product was isolated by isoelectric precipitation at pH=3-5. The product was collected via vacuum filtration, washed with water and air dried to afford the title compound (0.052 g, 0.14 mmol, 40%).

¹H NMR (CDCl₃) δ: 8.07 (s,1H), 7.53 (d, 2H, J=7.9 Hz), 7.33-7.25 (m, 1H), 7.07 (t, 1H, J=7.3 Hz), 4.26 (q, 2H, J=7.1 Hz ), 4.19-4.06 (m, 2H), 2.87-2.75 (m, 1H), 2.45-2.24 (m, 5H), 1.87-1.75 (m, 4H), 1.34-1.20 (m, 6H) ppm.MS(ES) for C₂₀H₂₅ClN₂O₆5 [M+H]⁺/—Cl: 375.04; [M−H]⁻/—Cl: 373.11

### EXAMPLE 79

### Synthesis of (1S*,2S*,4R*,5R*,6S*) 2-Amino-4-(m-methoxy) phenylcarbamoylmethyl bicyclo[3.1.0]hexane-2,6-dicarboxylic acid

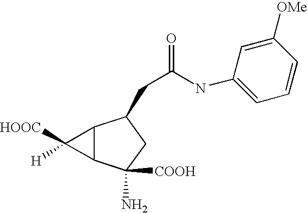

### A. 4(Z/E),(1S*,2S*,5R*,6S*) Diethyl 2-tert-butyloxy-carbonylamino-4-(m-methoxy)phenylcarbamoylmethylene bicyclo[3.1.01hexane-2,6-dicarboxylate

A room temperature solution of the product of Example 74B (0.5 g, 1.25 mmol) in CH₂Cl₂ was treated consecutively with oxalyl chloride (0.239 g, 1.89 mmol) and a catalytic amount of DMF. After the reaction mixture was stirred for one hour, the solvent was removed in vacuo. The crude product was reconstituted in CH₂Cl₂ and then a solution of m-anisidine (0.307 g, 2.5 mmol) was added dropwise. The reaction mixture was allowed to stir at room temperature until the reaction was judged complete by TLC. The resulting mixture was concentrated to dryness and the crude product purified by filtration through a short pad of SiO₂ to afford the title compounds (0.505 g, 1.0 mmol) 80% as a mixture of diastereoisomers which were used in the next step without further separation.

### B. (1S*,2S*,4R*,5R*,6S*) Diethyl 2-tert-butyloxy carbonylamino-4-(m-methoxy)phenylcarbamoylmethyl bicyclo[3.1.01hexane-2,6-dicarboxylate

A room temperature solution of the product of Example 79A (0.505 g, 1.0 mmol) and 10% Pd/C (0.025 g, 5 wt %) in 95% EtOH was vigorous stirred under an hydrogen atmosphere (1 atm) over night. The catalyst was removed via vacuum filtration through celite and the filtrate concentrated in vacuo to afford the title compound (0.354 g, 0.70 mmol, 70%).

### C. (1S*,2S*,4R*,5R*,6S*) Diethyl 2-Amino-4-(m-methoxy) phenylcarbamoyl methylbicyclo[3.1.01hexane-2,6-dicarboxylate

The title compound was prepared as in Example 74E to give the product

¹H NMR (CDCl₃)δ: 8.22(s, 1H, NHCO), 7.33-7.12 (m,2H), 6.98 (brd, 2H), 6.61 (dd, 1H, J=2.2 and 8.0 Hz), 4.23 (q, 2H, J=7.1 Hz), 4.08 (q, 2H, J=7.1 Hz), 3.76 (s, 3H), 2.81-2.72 (m, 1H), 2.50-2.21 (m, 5H), 1.87-1.74 (m, 4H), 1.39-1.20 (dt, 6H) ppm. MS(ES) for C₂₁H₂₈N₂O₆ [M+H]⁺: 405.05; [M−H]⁻: 403.16

### D. (1S*,2S*,4R*,5R*,6S*) 2-Amino-4-(m-methoxy) phenylcarbamoylmethylbicyclo(3.1.0]hexane-2,6-dicarboxylic acid

A solution of the product of Example 79C (0.140 g, 0.34 mmol) in THF was treated with 1N NaOH (0.69 mmol) and stirred at room temperature over night. The solvent was removed in vacuo and the crude product reconstituted in distilled water and acidified with 1N HCl. The product was isolated by isoelectric precipitation at pH=3-5. The product was collected via vacuum filtration, washed with water and air dried to afford the title compound (0.085 g, 0.24 mmol, 70%).

¹H-NMR (D₂O+Pyr)δ: 6.93 (t, 1H, J=7.9 and 8.1 Hz); 6.75 (s, 1H), 6.67 (d, 1H, J=7.9 Hz); 6.38 (d, 1H, J=8.1 Hz); 3.43 (s, 3H); 2.48-2.31 (m, 3H), 1.99 (dd, 1H, J=3.4 and 3.5 Hz), 1.80-1.57 (m, 3H), 1.42 (t, 1H, J=3.0 Hz) ppm. ¹³C-NMR (D₂O+Pyr) δ: 179.8, 176.4, 173.4, 159.3, 138.6, 130.2, 113.8, 110.8, 106.8, 67.8, 55.4, 41.6, 38.4, 38.2, 33.5, 33.3, 26.0 ppm. MS(ES) for C₁₇H₂₀N₂O₆ [M+H]⁺: 348.99 [M−H])⁻: 347.08 Pf>2000 dec.

### EXAMPLE 80

### Synthesis of (1S*,2S*,4R*,5R*,6S*) 2-Amino-4-(m-methyl) phenylcarbamoylmethyl bicyclo[3.1.0]hexane-2,6-dicarboxylic acid

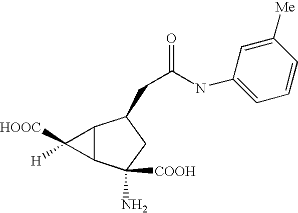

### A. 4(Z/E)(1S*,2S*,5R*,6S*) Diethyl 2-tert-butyloxycarbonyl amino-4-(m-methyl)phenylcarbamoylmethylene bicyclo[3.1.0]hexane-2,6-dicarboxylate

A room temperature solution of the product of Example 74B (0.5 g, 1.25 mmol) in CH₂Cl₂ was treated consecutively with oxalyl chloride (0.239 g, 1.89 mmol) and a catalytic amount of DMF. After the reaction mixture was stirred for one hour, the solvent was removed in vacuo. The crude product was reconstituted in CH₂Cl₂ and then a solution of m-toluidine (0.267 g, 2.5 mmol) was added dropwise. The reaction mixture was allowed to stir at room temperature until the reaction was judged complete by TLC. The resulting mixture was concentrated to dryness and the crude product purified by filtration through a short pad of SiO₂ to afford the title compounds (0.399 g, 0.81 mmol) 65% as a mixture of diastereoisomers which were used in the next step without further separation.

### B. (1S*,2S*,4R*,5R*,6S*) Diethyl 2-tert-butyloxy carbonylamino-4-(m-methyl)phenylcarbamoylmethyl bicyclo[3.1.0]hexane-2,6-dicarboxylate

A room temperature solution of the product of Example 80A (0.309 g, 0.81 mmol) and 10% Pd/C (0.015 g, 5 wt %) in 95% EtOH was vigorous stirred under an hydrogen atmosphere (1 atm) over night. The catalyst was removed via vacuum filtration through celite and the filtrate concentrated in vacuo to afford the title compound (0.217 g, 0.44 mmol, 70%).

### C. (1S*,2S*,4R*,5R*,6S*) Diethyl 2-Amino-4-(m-methyl) phenylcarbamoyl methylbicyclo(3.1.0]hexane-2,6-dicarboxylate

The title compound was prepared as in Example 74E to give the product in 64% yield

¹H NMR (CDCl₃)δ: 8.30(s, 1H, NHCO), 7.50-7.24 (m, 3H), 7.99 (d, 1H, J=7.4 Hz), 4.36 (q, 2H, J=7.1 Hz), 4.26-4.15 (m, 2H), 2.93-2.85 (m, 1H), 2.62-2.34 (m, 8H), 1.99-1.95 (m, 3H), 1.87 (t, 1H, J=3.0 Hz), 1.51-1.29 (dt, 6H) ppm. MS(ES) for C₂₁H₂₈N₂O₅ [M+H]⁺: 389.05; [M−H]⁻: 387.17

### D. (1S*,2S*,4R*,5R*,6S*) 2-Amino-4-(m-methyl) phenylcarbamoylmethylbicyclo[3.1.0]hexane-2,6-dicarboxylic acid

A solution of the product of Example 80C (0.100 g, 0.25 mmol) in THF was treated with 1N NaOH (0.51 mmol) and stirred at room temperature over night. The solvent was removed in vacuo and the crude product reconstituted in distilled water and acidified with 1N HCl. The product was isolated by isoelectric precipitation at pH=3-5. The product was collected via vacuum filtration, washed with water and air dried to afford the title compound (0.040 g, 0.12 mmol, 47%).

¹H-NMR (MeOD+Pyr)δ: 7.44-7.34 (m, 2H); 6.75 (s, 1H), 6.67 (d, 1H, J=7.9 Hz); 6.38 (d, 1H, J=8.1 Hz); 3.43 (s, 3H); 2.48-2.31 (m, 3H), 1.99 (dd, 1H, J=3.4 and 3.5 Hz), 1.80-1.57 (m, 3H), 1.42 (t, 1H, J=3.0 Hz) ppm. 13C-NMR (MeOD+Pyr)δ: 75.8, 175.5, 1721.8, 139.9, 139.6, 129.6, 125.6, 121.5, 118.1, 68.5, 42.6, 39.5, 38.6, 36.0, 35.3, 24.3, 21.6 ppm. MS(ES) for C₁₇H₂₀N₂O₅ [M+H]⁺: 333.0 [M−H]⁻: 331.10

### EXAMPLE 81

### Synthesis of (1S*,2S*,4R*,5R*,6S*) 2-Amino-4-(m-trifluoro methyl)phenylcarbamoylmethyl bicyclo[3.1.0)hexane-2,6-dicarboxylic acid

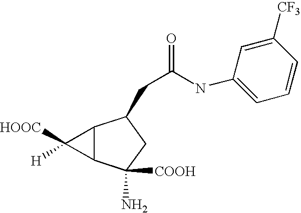

### A. 4(Z/E),(1S*,2S*,5R*,6S*) Diethyl 2-tert-butyloxy-carbonylamino-4-(m-trifluoromethyl)phenylcarbamoylmethylene bicyclo[3.1.0]hexane-2,6-dicarboxylate

A room temperature solution of the product of Example 74B (0.5 g, 1.25 mmol) in CH₂Cl₂ was treated consecutively with oxalyl chloride (0.239 g, 1.89 mmol) and a catalytic amount of DMF. After the reaction mixture was stirred for one hour, the solvent was removed in vacuo. The crude product was reconstituted in CH₂Cl₂ and then a solution of m-trifluoromethylaniline (0.402 g, 2.5 mmol) was added dropwise. The reaction mixture was allowed to stir at room temperature until the reaction was judged complete by TLC. The resulting mixture was concentrated to dryness and the crude product purified by filtration through a short pad of SiO₂ to afford the title compounds (0.462 g, 0.85 mmol) 68% as a mixture of diastereoisomers which were used in the next step without further separation.

### B. (1S*,2S*,4R*,5R*,6S*) Diethyl 2-tert-butyloxycarbonyl amino-4-(m-trifluoromethyl)phenylcarbamoyl methyl bicyclo[3.1.0]hexane-2,6-dicarboxylate

A room temperature solution of the product of Example 81A (0.462 g, 0.85 mmol) and 10% Pd/C (0.023 g, 5 wt %) in 95% EtOH was vigorous stirred under an hydrogen atmosphere (1 atm) over night. The catalyst was removed via vacuum filtration through celite and the filtrate concentrated in vacuo to afford the title compound (0.347 g, 0.64 mmol, 70%).

MS(ES) for C₂₆H₃₃F₃N₂O₇ [M+H]⁺: 543.10 [M−H]⁻: 541.23

### C. (1S*,2S*,4R*,5R*,6S*) Diethyl 2-Amino-4-(m-trifluoro methyl)phenylcarbamoyl methylbicyclo[3.1.0]hexane-2,6-dicarboxylate

The title compound was prepared as in Example 74E to give the product in 56% yield. MS(ES) for C₂₁H₂₅F₃N₂O₅ [M+H]⁺: 443.2.

### D. (1S*,2S*,4R*,5R*,6S*) 2-Amino-4-(m-trifluoromethyl)phenylcarbamoylmethylbicyclo [3.1.0]hexane-2,6-dicarboxylic acid

A solution of the product of Example 81C (0.140 g, 0.31 mmol) in THF was treated with 1N NaOH (0.8 mmol) and stirred at room temperature over night. The solvent was removed in vacuo and the crude product reconstituted in distilled water and acidified with 1N HCl. The product was isolated by isoelectric precipitation at pH=3-5. The product was collected via vacuum filtration, washed with water and air dried to afford the title compound (0.110 g, 0.28mmol, 90%). MS(ES) for C₁₇H₁₇F₃N₂O₅ [M+H]⁺: 387.04.

### EXAMPLE 82

### Synthesis of (1S*,2S*,4R*,5R*,6S*) 2-Amino-4-(p-methoxy) phenylcarbamoylmethyl bicyclo[3.1.0]hexane-2,6-dicarboxylic acid

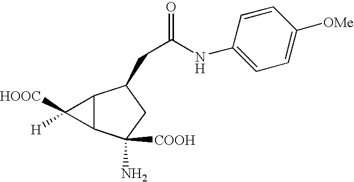

### A. 4(Z/E), (1S*,2S*,5R*,6S*) Diethyl 2-tert-butyloxy carbonylamino-4-(p-methoxy)phenylcarbamoylmethylene bicyclo[3.1.0]hexane-2,6-dicarboxylate

A room temperature solution of the product of Example 74B (0.5 g, 1.25 mmol) in CH₂Cl₂ was treated consecutively with oxalyl chloride (0.239 g, 1.89 mmol) and a catalytic amount of DMF. After the reaction mixture was stirred for one hour, the solvent was removed in vacuo. The crude product was reconstituted in CH₂Cl₂ and then a solution of p-anisidine (0.307 g, 2.5 mmol) was added dropwise. The reaction mixture was allowed to stir at room temperature until the reaction was judged complete by TLC. The resulting mixture was concentrated to dryness and the crude product purified by filtration through a short pad of SiO₂ to afford the title compounds (0.252 g, 0.5 mmol) 40% as a mixture of diastereoisomers which were used in the next step without further separation.

### B. (1S*,2S*,4R*,5R*,6S*) Diethyl 2-tert-butyloxycarbonylamino-4-(p-methoxy)phenylcarbamoyl methylbicyclo[3.1.0]hexane-2,6-dicarboxylate

A room temperature solution of the product of Example 82A (0.252 g, 0.5 mmol) and 10% Pd/C (0.013 g, 5 wt %) in 95% EtOH was vigorous stirred under an hydrogen atmosphere (1 atm) over night. The catalyst was removed via vacuum filtration through celite and the filtrate concentrated in vacuo to afford the title compound (0.164 g, 0.32 mmol, 65%).

### C. (1S*,2S*,4R*,5R*,6S*) Diethyl 2-Amino-4-(p-methoxy) phenylcarbamoyl methylbicyclo[3.1.0]hexane-2,6-dicarboxylatte

The title compound was prepared as in Example 74E to give the product in 30% yield.

¹H NMR (CDCl₃)δ: 7.72 (s, 1H), 7.37 (d, 2H, J=8.9 Hz), 6.81 (d, 2H, J=8.9 Hz), 4.28-4.06 (dq, 4H), 3.77 (s, 3H), 3.68-2.72 (m, 1H), 2.49-2.44 (m, 2H), 2.34(dd, 1H, J=2.6 and 5.8 Hz), 2.15-1.96 (m, 4H), 1.68 (t, 1H, J=3.0 Hz), 1.55 (dd, 1H, J=8.1 and 14.0 Hz), 1.35-1.20 (dt, 6H) ppm

### D. (1S*,2S*,4R*,5R*,6S*) 2-Amino-4-(p-methoxy) phenylcarbamoylmethylbicyclo[3.1.0]hexane-2,6-dicarboxylic acid

A solution of the product of Example 82C (0.025 g, 0.06 mmol) in THF was treated with 1N NaOH (0.12 mmol) and stirred at room temperature over night. The solvent was removed in vacuo and the crude product reconstituted in distilled water and acidified with 1N HCl. The product was isolated by isoelectric precipitation at pH=3-5. The product was collected via vacuum filtration, washed with water and air dried to afford the title compound (0.008 g, 0.02 mmol, 38%).

¹H-NMR (D₂O+KOD)δ: 7.15 (d, 2H, J=8.8 Hz); 6.82 (d, 2H, J=8.9 Hz); 3.64 (s, 3H); 2.52-2.25 (m, 3H); 1.91-1.80 (m, 1H); 1.76 (d, 1H, J=14.5 Hz), 1.38 (m, 1H), 1.30-1.24 (m, 2H) ppm. ¹³C-NMR (D₂O+KOD) δ: 184.5, 182.4, 174.8, 130.7, 124.8, 114.9, 66.6, 56.1, 42.0, 41.4, 38.4, 35.9, 32.2, 25.5 ppm

### EXAMPLE 83

### Synthesis of (1S*,2S*,4R*,5R*,6S*) 2-Amino-4-(p-methyl) phenylcarbamoylmethyl bicyclo(3.1.0]hexane-2,6-dicarboxylic acid

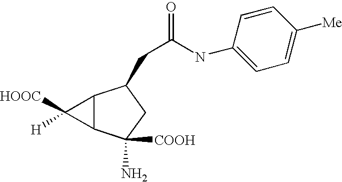

### A. 4(Z/E), (1S*,2S*,5R*,6S*) Diethyl 2-tert-butyloxy carbonylamino-4-(p-methyl)phenylcarbamoylmethylene bicyclo[3.1.01hexane-2,6-dicarboxylate

A room temperature solution of the product of Example 74B (0.5 g, 1.25 mmol) in CH₂Cl₂ was treated consecutively with oxalyl chloride (0.239 g, 1.89 mmol) and a catalytic amount of DMF. After the reaction mixture was stirred for one hour, the solvent was removed in vacuo. The crude product was reconstituted in CH₂Cl₂ and then a solution of p-toluidine (0.270 g, 2.5 mmol) was added dropwise. The reaction mixture was allowed to stir at room temperature until the reaction was judged complete by TLC. The resulting mixture was concentrated to dryness and the crude product purified by filtration through a short pad of SiO₂ to afford the title compounds (0.220 g, 0.45 mmol) 36% as a mixture of diastereoisomers which were used in the next step without further separation.

### B. (1S*,2S*,4R*,5R*,6S*) Diethyl 2-tert-butyloxy carbonylamino-4-(p-methyl)phenylcarbamoylmethyl bicyclo[3.1.0]hexane-2,6-dicarboxylate

A room temperature solution of the product of Example 83A (0.220 g, 0.45 mmol) and 10% Pd/C (0.011 g, 5 wt %) in 95% EtOH was vigorous stirred under an hydrogen atmosphere (1 atm) over night. The catalyst was removed via vacuum filtration through celite and the filtrate concentrated in vacuo to afford the title compound (0.137 g, 0.34 mmol, 65%).

MS(ES) for C₂₆H₃₆N₂O₇ [M+H]⁺: 489.25 [M=H]⁻: 487.09.

### C. (1S*,2S*,4R*,5R*,6S*) Diethyl 2-Amino-4-(p-methyl) phenylcarbamoyl methylbicyclo[3.1.0]hexane-2,6-dicarboxylic acid

The title compound was prepared as in Example 74E to give the product in 50% yield.

¹H NMR (CDCl₃)δ: 8.04 (s, 1H), 7.41 (d, 1H, J=8.4 Hz), 7.09 (d, 1H, J=8.3 Hz), 4.26 (q, 2H, J=7.1 Hz), 4.11 (q, 2H, J=7.1 Hz), 2.86-2.67 (m, 1H), 2.50-2.23 (m, 7H), 1.87-1.74 (m, 4H), 1.37-1.21 (dt, 6H) ppm.

### D. (1S*,2S*,4R*,5R*,6S*) 2-Amino-4-(p-methyl) phenylcarbamoylmethylbicyclo[3.1.01 hexane-2,6-dicarboxylic acid

A solution of the product of Example 83C (0.055 g, 0.14 mmol) in THF was treated with 1N NaOH (0.28 mmol) and stirred at room temperature over night. The solvent was removed in vacuo and the crude product reconstituted in distilled water and acidified with 1N HCl. The product was isolated by isoelectric precipitation at pH=3-5. The product was collected via vacuum filtration, washed with water and air dried to afford the title compound (0.026 g, 0.07mmol, 55%).

¹H-NMR (D₂O+KOD) δ: 7.26-7.14 (AA′BB′′, 4H), 2.58-2.30 (m, 3H), 2.24 (s, 3H); 2.02 (dd, 1H, J=3.0 and 6.1 Hz), 1.87 (d, 1H, J=14.4 Hz), 1.66 (dd, 1H, J=2.9 and 6.1 Hz), 1.46-1.19 (m, 2H) ppm. ¹³C-NMR (D₂O+KOD) δ: 181.9, 174.3, 137.3, 135.7, 129.5, 122.3, 66.0, 41.7, 40.9, 37.8, 35.4, 31.8, 24.9, 19.9 ppm

### EXAMPLE 84

### Synthesis of (1S*,2S*,4R*,5R*,6S*) 2-Amino-4-(p-trifluoro methyl)phenylcarbamoylmethyl bicyclo[3.1.0]hexane-2,6-dicarboxylic acid

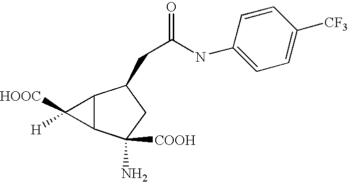

### A. 4(Z/E), (1S*,2S*,5R*,6S*) Diethyl 2-tert-butyloxy carbonylamino-4-(p-trifluoromethyl)phenylcarbamoylmethylene bicyclo[3.1.0]hexane-2,6-dicarboxylate

A room temperature solution of the product of Example 74B (0.5 g, 1.25 mmol) in CH₂Cl₂ was treated consecutively with oxalyl chloride (0.239 g, 1.89 mmol) and a catalytic amount of DMF. After the reaction mixture was stirred for one hour, the solvent was removed in vacuo. The crude product was reconstituted in CH₂Cl₂ and then a solution of p-trifluoromethylaniline (0.290 g, 2.5 mmol) was added. The reaction mixture was allowed to stir at room temperature until the reaction was judged complete by TLC. The resulting mixture was concentrated to dryness and the crude product purified by filtration through a short pad of SiO₂ to afford the title compound (0.340 g, 0.62 mmol) 50% as a mixture of diastereoisomers which were used in the next step without further separation.

### B. (1S*,2S*,4R*, 5R*,6S*) Diethyl 2-tert-butyloxy carbonylamino-4-(p-trifluoromethyl)phenylcarbamoylmethyl bicyclo[3.1.0]hexane-2,6-dicarboxylate

A room temperature solution of the product of Example 84A (0.34 g, 0.62 mmol) and 10% Pd/C (0.017 g, 5 wt %) in 95% EtOH was vigorous stirred under an hydrogen atmosphere (1 atm) over night. The catalyst was removed via vacuum filtration through celite and the filtrate concentrated in vacuo to afford the title compound (0.320 g, 0.58 mmol, 93 %). MS(ES) for C₂₆H₃₁F₃N₂O₇ [M+H]⁺: 545.24 [M−H]⁻: 543.27

### C. (1S*,2S*,4R*,5R*,6S*) Diethyl 2-Amino-4-(p-trifluoro methyl)phenylcarbamoyl methylbicyclo[3.1.0]hexane-2,6-dicarboxylate

The title compound was prepared as in Example 74E to give the product in 54% yield

¹H NMR (CDCl₃) δ: 8.50 (s, 1H), 7.797.51 (m, 4H), 4.34-4.01 (m, 4H), 2.97-2.17 (m, 6H), 1.98-1,65 (m, 4H), 1.38-1.18 (m, 6H) ppm. MS(ES) for C₂₁H₂₅F₃N₂O₅ [M+H]⁺: 443.01; [M−H]⁻: 441.15

### D. (1S*,2S*,4R*,5R*,6S*) 2-Amino-4-(p-trifluoro methyl)phenylcarbamoylmethylbicyclo[3.1.0]hexane-2,6-dicarboxylic acid

A solution of the product of Example 84C (0.067 g, 0.15 mmol) in THF was treated with 1N NaOH (0.30 mmol) and stirred at room temperature over night. The solvent was removed in vacuo and the crude product reconstituted in distilled water and acidified with 1N HCl. The product was isolated by isoelectric precipitation at pH=3-5. The product was collected via vacuum filtration, washed with water and air dried to afford the title compound (0.033 g, 0.08 mmol, 57%).

¹H-NMR (D₂O+KOD) δ: 7.65 (d, 2H, J=8.6 Hz); 7.54 (d, 2H, J=8.8 Hz); 2.65-2.40 (m, 3H); 2.04 (d, 1H, J=3.1 and 6.2 Hz); ); 1.89 (d, 1H, J=14.4 Hz), 1.69 (d, 1H, J=2.9 and 5.9 Hz); 1.48-1.35 (m, 2H) ppm. MS(ES) for C₁₇H₁₇F₃N₂O₅ [M+H]⁺: 387.04 [M−H]⁻: 385.07

### EXAMPLE 85

### Synthesis of (1S*,2S*,4R*,5R*,6S*) 2-Amino-4-(m-chloro) phenylcarbamoylmethyl bicyclo[3.1.0]hexane-2,6-dicarboxylic acid

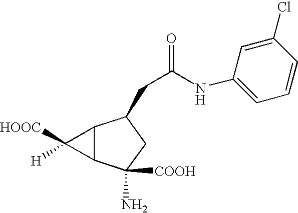

### A. 4(Z/E),(1S*,2S*,5R*,6S*) Diethyl 2-tert-butyloxy carbonylamino-4-(m-chloro)phenylcarbamoylmethylene bicyclo[3.1.0]hexane-2,6-dicarboxylate

A room temperature solution of the product of Example 74B (0.5 g, 1.25 mmol) in CH₂Cl₂ was treated consecutively with oxalyl chloride (0.239 g, 1.89 mmol) and a catalytic amount of DMF. After the reaction mixture was stirred for one hour, the solvent was removed in vacuo. The crude product was reconstituted in CH₂Cl₂ and then a solution of m-chloroaniline(0.290 g, 2.5 mmol) was added dropwise. The reaction mixture was allowed to stir at room temperature until the reaction was judged complete by TLC. The resulting mixture was concentrated to dryness and the crude product purified by filtration through a short pad of SiO₂ to afford the title compound (0.340 g, 0.62 mmol) 50% as a mixture of diastereoisomers which were used in the next step without further separation.

### B. (1S*,2S*,4R*,5R*,6S*) Diethyl 2-tert-butyloxy carbonylamino-4-(m-chloro)phenylcarbamoylmethyl bicyclo[3.1.0)hexane-2,6-dicarboxylate

A room temperature solution of the product of Example 85A (0.34 g, 0.62 mmol) and 10% Pd/C (0.017 g, 5 wt %) in 95% EtOH was vigorous stirred under an hydrogen atmosphere (1 atm) over night. The catalyst was removed via vacuum filtration through celite and the filtrate concentrated in vacuo to afford the title compound.

### C. (1S*,2S*,4R*,5R*,6S*) Diethyl 2-Amino-4-(m-chloro) phenylcarbamoyl methylbicyclo[3.1.0]hexane-2,6-dicarboxylic acid

The title compound was prepared as in Example 74E to give the product.

¹H NMR (CDCl₃) δ: 8.27 (bs, 1H), 7.52 (m, 2H), 7.27 (m, 2H), 7.04 (m, 1H), 4.22 (q, 2H, J=7.1 Hz), 4.08 (q, 2H), 2.89-2.72 (m, 1H), 2.51-2.28 (m, 5H), 1.85-1.73 (m, 4H), 1.25 (dt, 6H) ppm. MS(ES) for C₂₀H₂₅ClN₂O₅ [M+H]⁺/—Cl: 375.03; [M−H]⁻/—Cl: 373.15

### D. (1S*,2S*,4R*,5R*,6S*) 2-Amino-4-(m-chloro)phenyl carbamoylmethylbicyclo[3.1.0]hexane-2,6-dicarboxylic acid

A solution of the product of Example 85C (0.067 g, 0.15 mmol) in THF was treated with 1N NaOH (0.30 mmol) and stirred at room temperature over night. The solvent was removed in vacuo and the crude product reconstituted in distilled water and acidified with 1N HCl. The product was isolated by isoelectric precipitation at pH=3-5. The product was collected via vacuum filtration, washed with water and air dried to afford the title compound MS(ES) for C₁₆H₁₇ClN₂O₅ [M+H]⁺/—Cl: 319.18 [M−H]⁻/—Cl: 317.23

¹³C-NMR (D₂O+Pyr) δ: 178.5, 175.2, 171.8, 136.7, 136.2, 128.0, 123.7, 123.1, 119.7, 66.8, 59.33, 40.6, 37.4, 37.2, 32.6, 32.2, 25.1 ppm.

### EXAMPLE 86

Synthesis of (1S*,2S*,4R*,5R*,6S*) 2-Amino-4-carboxymethyl bicyclo[3.1.0]hexane-2,6-dicarboxylic acid

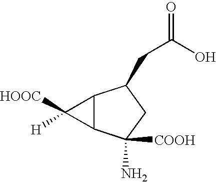

### A. 4(Z/E),(1S*,2S*,5R*,6S*) Diethyl 2-tert-butyloxy carbonylamino-4-methoxycarbonylmethylene bicyclo[3.1.0]hexane-2,6-dicarboxylate

A room temperature solution of the product of Example 74B (0.5 g, 1.25 mmol) in CH₂Cl₂ was treated consecutively with oxalyl chloride (0.239 g, 1.89 mmol) and a catalytic amount of DMF. After the reaction mixture was stirred for one hour, the solvent was removed in vacuo. The crude product was reconstituted in CH₂Cl₂ and then a solution of trifluoromethylaniline was added. The reaction mixture was allowed to stir at room temperature until the reaction was judged complete by TLC. The resulting mixture was concentrated to dryness and the crude product purified by filtration through a short pad of SiO₂ to afford the title compound as a mixture of diastereoisomers which were used in the next step without further separation.

### B. (1S*,2S*,4R*,5R*,6S*) Diethyl 2-tert-butyloxy carbonylamino-4-methoxycarbonylmethyl bicyclo[3.1.0]hexane-2,6-dicarboxylate

A room temperature solution of the product described above (2 g, 5.0 mmol) and 10% Pd/C (0.250 g, 5 wt %) in 95% EtOH was vigorous stirred under an hydrogen atmosphere (1 atm) over night. The catalyst was removed via vacuum filtration through celite and the filtrate concentrated in vacuo to afford the title compound (1.7 g, 4.2 mmol, 84%). MS(ES) for C₁₀H₁₃NO₆ [M+H]⁺: 244.16 [M−H]⁻: 242.19.

### C. (1S*,2S*,4R*,5R*,6S*) Diethyl 2-Amino-4-methoxycarbonyl methylbicyclo[3.1.0] hexane-2,6-dicarboxylic acid

The title compound was prepared as example 74E to give the product in 70% ¹H NMR (CDCl₃) δ: 4.23-4.00 (dq, 4H), 3.60 (s, 3H), 2.70-2.60 (m, 1H), 2.51-2.33 (m, 2H), 2.26 (dd, 1H, J=3.0 and 6.2 Hz), 2.07 (d, 1H, J=14.8 Hz), 1.83 (dd, 1H, J=3.0 and 6.1 Hz), 1.74 (brs, 2H), 1.67 (t, 1H, J=3.0 Hz), 1.43 (dd, 1H, J=8.3 and 14.8 Hz), 1.30 1.15 (m, 6H). ppm

### D. (1S*,2S*,4R*,5R*,6S*) 2-Amino-4-carboxymethyl bicyclo[3.1.0]hexane-2,6-dicarboxylic acid

A solution of the product of Example 86C (0.180 g, 0.6 mmol) in THF was treated with 1N NaOH (1.2 mmol) and stirred at room temperature over night. The solvent was removed in vacuo and the crude product reconstituted in distilled water and acidified with 1N HCl. The product was isolated by ion exchange chromatography, washed with water and air dried to afford the title compound (0.052 g, 0.21 mmol, 40%).

¹H-NMR (D₂O+Pyr) δ: 2.89-2.37 (m, 3H), 2.08 (dd, 1H, J=3.1 and 6.1 Hz), 1.93-1.62 (m, 3H), 1.50 (t, 1H, J=3.1 Hz) ppm. ¹³C-NMR (D₂O+Pyr) δ: 183.9, 182.6, 179.0, 70.4, 45.1, 41.5, 41.2, 36.4, 36.0, 28.7 ppm. MS(ES) for C₁₀H₁₃NO₆ [M+H]⁺: 244.16 [M−H]⁻: 242.19.

### EXAMPLE 87

Synthesis of (1S*,2S*,4R*,5R*,6S*) 2-Amino-4-benzoxazol-2-yl-methylbicyclo[3.1.0]hexane-2,6-dicarboxylic acid

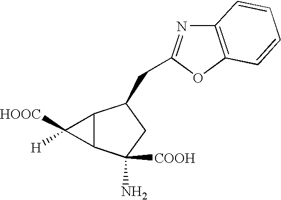

### A. 4(Z/E), 1S*,2S*,5R*,6S* Diethyl 2-tert-butyloxy-carbonylamino-4-(benzoxazol-2-yl)methylene bicyclo[3.1.0]-hexane-2,6-dicarboxylate

To a 0° C. solution of diethyl benzoxazol-2-ylmethylphosphonate (previously prepared) 0.454 1.68 mmol in anhydrous THF, was added 1M NaHMDS (1.68 mmol) by dropwise addition. The resulting reaction mixture was stirred at 0° C. for 1 h, after which time a solution of (1S*,2S*,5R*,6R*) diethyl2-N-tert-butyloxycarbonylamino-4-oxobicyclo[3.1.0] hexane-2,6-dicarboxylate (previously disclosed U.S. Pat. No. 5,958,960 example 5a) in THF was added dropwise. The reaction mixture was allowed to room temperature as it stirred 4 h. The reaction was quenched with saturated aqueous NH₄Cl solution and the product extracted with ethyl acetate. The combined organic extracts were dried over sodium sulfate, concentrated to dryness in vacuo, and purified by SiO₂ chromatography. (EtOAc/hexanes. 3:7) to afford 0.380 g (1.12 mmol, 72%) of the title compound as a (1:1.5) mixture of diastereoisomers 1:1.5 which were used in the next step without further separation. MS(ES) for C₂₅H₃₀N₂O₇ [M+H]⁺: 471.3.

### B. (1S*,2S*,4R*,5R*,6S*) Diethyl 2-tert-butyloxycarbonyl-amino-4-(benzoxazol-2-yl)methylbicyclo[3.1.0]hexane-2,6-dicarboxylate

A room temperature solution of the product of 87A (0.380 g, 0.8 mmol) and 10% Pd/C (0.020 g, 5 wt %) in 95% EtOH was vigorous stirred under an hydrogen atmosphere (1 atm) over night. The catalyst was removed via vacuum filtration through celite and the filtrate concentrated in vacuo to afford the title compound (0.275 g, 0.58 mmol, 72%). MS(ES) for C₂₅H₃₂N₂O₇ [M+H]⁺: 473.07.

### C. (1S*,2S*,4R*,5R*,6S*) Diethyl 2-Amino-4-(benzoxazol-2-yl)methylbicyclo [3.1.0]hexane-2,6-dicarboxylic acid

The title compound was prepared as example 74E to give the product in 53%.

¹H NMR (CDCl₃) δ: 7.66-7.62 (m, 1H), 7.47-7.43 (m, 1H), 7.31-7.24 (m, 2H), 4.26 (q, 2H, J=7.1 Hz), 4.08 (q, 2H, J=7.1 Hz), 3.09-2.90 (m, 3H), 2.37 (dd, 1H, J=2.9 and 6.0 Hz), 2.25 (d, 1H, J=14.8 Hz), 2.01 (d, 1H, J=3.1 and 5.9 Hz), 1.79-1.74 (m, 3H), 1.53 (dd, 1H, J=7.9 and 14.8 Hz ), 1.33 (t, 3H, J=7.1 Hz), 1.22 (t, 3H, J=7.1 Hz). ppm. MS(ES) for C₂₀H₂₄N₂O₇5 [M+H]⁺: 373.18.

### D. (1S*,2S*,4R*,5R*,6S*) 2-Amino-4-benzoxazol-2-yl-methylbicyclo[3.1.0]hexane-2,6-dicarboxylic acid

A solution of the product of Example 87C (0.080 g, 0.21 mmol) in THF was treated with 1N NaOH (0.43 mmol) and stirred at room temperature over night. The solvent was removed in vacuo and the crude product reconstituted in distilled water and acidified with 1N HCl. The product was isolated by isoelectric precipitation at pH=3-5. The product was collected via vacuum filtration, washed with water and air dried to afford the title compound (0.040 g, 0.12mmol, 59%).

¹H-NMR (D₂O+KOD)δ: 7.60-7.52 (m, 2H), 7.33-7.29 (m, 2H), 3.04-2.98 (m, 2H), 2.68-2.56 (m, 1H), 2.04 (dd, 1H, J=3.2 and 6.4 Hz), 1.92 (d, 1H, J=14.6 Hz), 1.74-1.71 (m, 1H), 1.48-1.45 (m, 2H)ppm. ¹H-NMR (D₂O+KOD) δ: 183.3, 181.2, 167.6, 125.0, 124.5, 118.5, 110.8, 66.1, 41.1, 38.6, 35.7, 33.2, 32.0, 25.0 ppm. MS(ES) for C₁₆H₁₆N₂O₅ [M+H]⁺: 317.02.

### EXAMPLE 88

### Synthesis of (1S*,2S*,4R*,5R*,6S*) 2-Amino-4-benzotiazol-2-yl-methylbicyclo[3.1.0]hexane-2,6-dicarboxylic acid

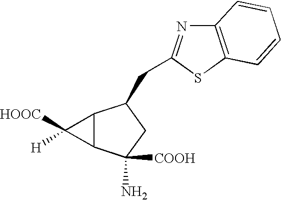

### A. 4(Z/E), (1S*,2S*,5R*,6S*) Diethyl 2-tert-butyloxycarbonylamino-4-(benzotiazol-2-yl)methylene bicyclo[3.1.0)hexane-2,6-dicarboxylate

To a 0° C. solution of diethyl benzotiazol-2-yl-methylphosphonate (previously prepared) 0.360 g, 1.26 mmol in anhydrous THF, was added 1M NaHMDS (1.26 mmol) by dropwise addition. The resulting reaction mixture was stirred at 0°C. for 1 h, after which time a solution of (1S*,2S*,5R*,6R*) diethyl-2-N-tert-butyloxycarbonylamino-4-oxobicyclo[3.1.01] hexane-2,6-dicarboxylate (previously disclosed U.S. Pat. No. 5,958,960 example 5a) 0.3 g, 0.84 mmol in THF was added dropwise. The reaction mixture was allowed to room temperature as it stirred 4 h. The reaction was quenched with saturated aqueous NH₄Cl solution and the product extracted with ethyl acetate. The combined organic extracts were dried over sodium sulfate, concentrated to dryness in vacuo, and purified by SiO₂ chromatography. (EtOAc/hexanes. 3:7) to afford 0.30 g (0.61 mmol, 72%) of the title compound as a (1:1.5) mixture of diastereoisomers which were used in the next step without further separation. MS(ES) for C₂₅H₃₀N₂O₆S [M+H]⁺: 486.98 [M−H]: 485.08.

### B. (1S*,2S*,4R*,5R*,6S*) Diethyl 2-tert-butyloxycarbonylamino-4-(benzotiazol-2-yl)methyl bicyclo[3.1.0]hexane-2,6-dicarboxylate

A room temperature solution of the product of 88A (0.30 g, 0.61 mmol) and 10% Pd/C (0.016 g, 5 wt %) in 95% EtOH was vigorous stirred under an hydrogen atmosphere (1 atm) over night. The catalyst was removed via vacuum filtration through celite and the filtrate concentrated in vacuo to afford the title compound (0.250 g, 0.51 mmol, 61%). MS(ES) for C₂₅H₃₂N₂O₆S [M+H]⁺: 489.05.

### C. (1S*,2S*,4R*,5R*,6S*) Diethyl 2-Amino-4-(benzotiazol-2-yl)methylbicyclo [3.1.0] hexane-2,6-dicarboxylic acid

The title compound was prepared as example 74E to give the product in 90%.

¹H NMR (CDCl₃) δ: 7.95 (ddd, 1H, J=7.8, 1.2 and 0.5 Hz), 7.80(ddd, 1H), 7.47-7.25 (m, 2H), 4.28 (q, 2H, J=7.1 Hz), 4.08 (q, 2H, J=7.1 Hz), 3.31-3.09 (m, 2H), 2.9 (m, 1H), 2.39 (dd, 1H, J=2.9 and 6.2 Hz), 2.33 (d, 1H, J=14.7 Hz), 2.01 (d, 1H, J=3.0 and 5.9 Hz), 1.76-1.73 (m, 3H), 1.49 (dd, 1H, J=8.3 and 14.7 Hz ), 1.34 (t, 3H, J=7.1 Hz), 1.22 (t, 3H, J=7.1 Hz) ppm.

### D. 1S*,2S*,4R*,5R*,6S*) 2-Amino-4-benzoxazol-2-yl-methylbicyclo[3.1.0]hexane-2,6-dicarboxylic acid

A solution of the product of Example 88C (0.080 g, 0.20 mmol) in THF was treated with 1N NaOH (0.40 mmol) and stirred at room temperature over night. The solvent was removed in vacuo and the crude product reconstituted in distilled water and acidified with 1N HCl. The product was isolated by isoelectric precipitation at pH=3-5. The product was collected via vacuum filtration, washed with water and air dried to afford the title compound (0.048 g, 0.14mmol, 70%).

¹H-NMR (D₂O+KOD) δ: 7.87-7.77 (m, 2H), 7.46-7.29 (m, 2H), 3.21-2.99 (m, 2H), 2.67-2.56 (m, 1H), 2.03 (dd, 1H, J=3.2 and 6.4 Hz), 1.90 (d, 1H, J=14.6 Hz), 1.72-1.69 (m, 1H), 1.43-1.30 (m, 2H) ppm. ¹³C-NMR (D₂O+KOD) δ: 181.8, 180.4, 172.2, 150.3, 132.9, 124.9, 123.7, 120.6, 119.8, 64.6, 39.8, 39.4, 36.8, 34.2, 30.5, 23.6 ppm. MS(ES) for C₁₆H₁₆N₂O₄S [M+H]⁺: 332.94 [M−H]⁻: 331.03.

### EXAMPLE 89

### Synthesis of (1S*,2S*,4R*,5R*,6S*) 2-Amino-4-benzylcarbamoyl methylbicyclo[3.1.0]hexane-2,6-dicarboxylic acid

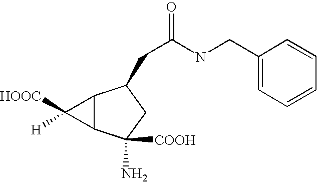

### A. 4(Z/E), (1S*,2S*,5R*,6S*) Diethyl 2-tert-butyloxy-carbonylamino-4-benzylcarbamoylmethylenebicyclo[3.1.01-hexane-2,6-dicarboxylate

A room temperature solution of the product of Example 74B (0.5 g, 1.25 mmol) in CH₂Cl₂ was treated consecutively with oxalyl chloride (0.239 g, 1.89 mmol) and a catalytic amount of DMF. After the reaction mixture was stirred for one hour, the solvent was removed in vacuo. The crude product was reconstituted in CH₂Cl₂ and then a solution of benzylamine (0.267 g, 2.5 mmol) was added dropwise. The reaction mixture was allowed to stir at room temperature until the reaction was judged complete by TLC. The resulting mixture was concentrated to dryness and the crude product purified by filtration through a short pad of SiO₂ to afford the title compound (0.350 g, 0.78 mmol) 62% as a mixture of diastereoisomers which were used in the next step without further separation. MS(ES) for C₂₆H₃₄N₂O₇ [M+H]⁺: 487.24 [M−H]⁻: 485.29.

### B. (1S*,2S*,4R*,5R*,6S*) Diethyl 2-tert-butyloxy-carbonylamino-4-benzylcarbamoyl methylbicyclo[3.1.0]hexane-2,6-dicarboxylate

A room temperature solution of the product of 89A (0.20 g, 0.41 mmol) and 10% Pd/C (0.015 g, 5 wt %) in 95% EtOH was vigorous stirred under an hydrogen atmosphere (1 atm) over night. The catalyst was removed via vacuum filtration through celite and the filtrate concentrated in vacuo to afford the title compound (0.150 g, 0.30 mmol, 75%). MS(ES) for C₂₆H₃₆N₂O₇ [M+H]⁺:

### C. (1S*,2S*,4R*,5R*,6S*) Diethyl 2-Amino-4-benzylcarbamoyl methylbicyclo[3.1.0]hexane-2,6-dicarboxylic acid

The title compound was prepared as example 74E to give the product.

### D. (1S*,2S*,4R*,5R*,6S*) 2-Amino-4-benzylcarbamoylmethyl-bicyclo [3.1.0]hexane-2,6-dicarboxylic acid

A solution of the product of Example 89C (0.150 g, 0.3 mmol) in THF was treated with 1N NaOH (0.6 mmol) and stirred at room temperature over night. The solvent was removed in vacuo and the crude product reconstituted in distilled water and acidified with 1N HCl. The product was isolated by isoelectric precipitation at pH=3-5. The product was collected via vacuum filtration, washed with water and air dried to afford the title compound (0.055 g, 0.16 mmol, 54%)

¹H-NMR (D₂O+KOD) δ: 7.33−7.24 (m, 5H), 4.69 (s, 2H), 2.55-2.21 (m, 3H, 2.99 (dd, 1H, J=3.2 and 5.9 Hz), 1.80 (d, 1H, J=14.4 Hz), 1.60 (dd, 1H, J=2.7 and 6.5 Hz), 1.43-1.32 (m, 2H) ppm. ¹³C-NMR (D₂O+KOD) δ: 185.2, 183.6, 177.2, 139.9, 130.6, 129.2, 129.0, 44.7, 42.9, 42.9, 39.7, 37.3, 33.6 ppm

We claim:

1. Use of a compound of the formula 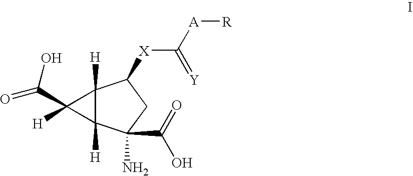
wherein:
X is CH₂, O, or NH;
Y is O, S, NH or H,H;
A is a bond O, N, (1-10C) alkyl, (2-10C) alkenyl or (2-10C) alkynyl;
R is hydrogen, (1-10C) alkyl, (2-10C) alkenyl, (3-6C) alkynyl, heterocyclyl, 1-naphthyl, 2-naphthyl, phenyl, 2-hydroxyphenyl, 3-hydroxyphenyl, 4-hydroxyphenyl, 3,5dihydroxyphenyl, 2-fluorophenyl, 3-fluorophenyl, 4-fluorophenyl 3,5-difluorophenyl, 2chlorophenyl, 3-chlorophenyl, 4chlorophenyl, 2,4-chlorophenyl, 3,4-dichlorophenyl, 3,5-dichlorophenyl, 2-methylphenyl, 3-methylphenyl, 4-methylphenyl, 2-methoxyphenyl, 3-methoxyphenyl, 4methoxyphenyl, 3-trifluoromethylphenyl, 4-trifluoromethylphenyl, 3carboxyphenyl, 4-carboxyphenyl or
3-(5′-tetrazoyl)phenyl;
or the group XC(Y)AR is 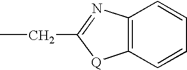
where Q is O, S or NH;
or a pharmaceutically acceptable metabolically labile ester or amide thereof;
or a pharmaceutically acceptable salt thereof, for the manufacture of a medicament for treatment or a neurological or psychiatric disorder.

2. The use of a compound (or salt thereof) of claim 1 wherein (1-10C) alkyl is methyl, ethyl, n-propyl, isopropyl, n-butyl, t-butyl, n-pentyl, isopentyl, n-hexyl, heptyl, n-octyl, nonyl or decyl; (2-10C) alkenyl is allyl, allenyl, 1-butenyl, 1-pentenyl, 3-nonenyl or 5-decenyl; (2-6C) alkynyl is ethynyl, propynyl, butynyl or pentynyl; aryl is phenyl, substituted phenyl or naphthyl and arylalkyl is benzyl, 2-nitro benzyl, or 1-phenylethyl, for the manufacture of a medicament for treatment or a neurological or psychiatric disorder.

3. The use of a compound (or salt thereof) of anyone of claims 1 or 2 wherein:
R is 2-methoxyphenyl, 3-methoxyphenyl, 4methoxyphenyl, 3methylphenyl, 4-methylphenyl, 2chlorophenyl, 3-chlorophenyl, 4chlorophenyl, 3-fluorophenyl, 2-fluorophenyl, 4fluorophenyl, 2,6-dichlorophenyl, 2,4-dichlorophenyl, 3,5-dichlorophenyl, 3,dichlorophenyl, methyl, 2-napthyl, 1-napthyl, 3-methylphenyl, phenyl, thiophenyl, 3-trifluoromethylphenyl, 2,3-dichlorophenyl, 1H-indol-3-yl cyclopropanyl, 1H-indol-2-yl, 4hydroxyphenyl, 3-hydroxyphenyl, 2-hydroxyphenyl, 4-biphenylyl, 1-isoquinolyl, 3-pyridinyl, 2-pyridinyl, 3,5-difluorophenyl, 4-pyridinyl, 2-methylphenyl, 2-quinoxalinyl, hydrogen, 3-carboxyphenyl, 1H-4-imidazoyl, 2-carboxyphenyl, 4carboxyphenyl, 2-trifluoromethylphenyl, benzyl, 4-trifluoromethylphenyl;
A is a bond, methyl, O, NH, ethenyl or ethynyl;
Y is O, S or H,H; and
X is CH₂, O or NH, for the manufacture of a medicament for treatment or a neurological or psychiatric disorder.

4. The compound (or salt thereof) of any one of claims 1-3 wherein R is heterocyclyl or 2-hydroxyphenyl, 3-hydroxyphenyl, 4-hydroxyphenyl, 3,5-dihydroxyphenyl, 2-fluorophenyl, 3-fluorophenyl, 4-fluorophenyl 3,5-difluorophenyl, 2-chlorophenyl, 3-chlorophenyl, 4-chlorophenyl, 2,4-chlorophenyl, 3,4-dichlorophenyl, 3,5-dichlorophenyl, 2-methylphenyl, 3-methylphenyl, 4-methylphenyl, 2-methoxyphenyl, 3-methoxyphenyl, 4-methoxyphenyl, 3-trifluoromethylphenyl, 4-trifluoromethylphenyl, 3-carboxyphenyl 4-carboxyphenyl.

5. The use of a compound (or salt thereof) of any one of claims 1-4 wherein R is 1H-indolyl, 2-napthyl, 3 chlorophenyl or 2-methoxyphenyl, for the manufacture of a medicament for treatment or a neurological or psychiatric disorder.

6. The use of a compound (or salt thereof) of any one of claims 1-5 wherein Y is O or S, for the manufacture of a medicament for treatment or a neurological or psychiatric disorder.

7. The use of a compound (or salt thereof) of any one of claims 1-6 wherein A is CH₂ or a bond, for the manufacture of a medicament for treatment or a neurological or psychiatric disorder.

8. The use of a compound of formula I which is (1S*, 2S*, 4S*, 5R*, 6S*) 2-amino-4-[3-3-chlorophenyl)ureido]bicyclo[3.1.0]hexane-2,6-dicarboxylic acid, for the manufacture of a medicament for treatment or a neurological or psychiatric disorder.

9. A compound which is selected from the group consisting of:
(1S*,2S*,4S*,5R*,6S*) 2-Amino-4-(3-phenylthioureido)-bicyclo[3.1.0]hexane-2,6-dicarboxylic acid,
(1S*, 2S*, 4S*, 5R*, 6S*) 2-Amino-4-[(thiophene-3-carbonyl)-amino]-bicyclo[3.1.0)hexane-2,6-dicarboxylic acid,
(1S*, 2S*, 4S*, 5R*, 6S*) 4-Acetylamino-2-amino-bicyclo[3.1.0]hexane-2,6-dicarboxylic acid,
(1S*, 2S*, 4S*, 5R*, 6S*) 2-Amino-4-benzoylamino-bicyclo [3.1.0]hexane-2,6-dicarboxylic acid,
(1S*, 2S*, 4S*, 5R*, 6S*) 2-Amino-4-(4-chloro-benzoylamino)-bicyclo[3.1.0]hexane-2,6-dicarboxylic acid,
(1S*, 2S*, 4S*, 5R*, 6S*) 2-Amino-4-(3-chloro-benzoylamino)-bicyclo[3.1.0]hexane-2,6-dicarboxylic acid,
(1S*, 2S*, 4S*, 5R*, 6S*) 2-Amino-4-(4-methoxy-benzoylamino)-bicyclo[3.1.0]hexane-2,6-dicarboxylic acid,
(1S*, 2S*, 4S*, 5R*, 6S*) 2-Amino-4-[(naphthalene-2-carbonyl)-amino]-bicyclo[3.1.0]hexane-2,6-dicarboxylic acid,
(1S*, 2S*, 4S*, 5R*, 6S*) 2-Amino-4-[(naphthalene-2-carbonyl)-amino]-bicyclo[3.1.0 ]hexane-2,6-dicarboxylic acid,
(1S*, 2S*, 4S*, 5R*, 6S*) 2-Amino-4-fluoro-benzoylamino)-bicyclo[3.1.0]hexane-2,6-dicarboxylic acid,
(1S*, 2S*, 4S*, 5R*, 6S*) 2-Amino-4-[(thiophene-2-carbonyl)amino]-bicyclo[3.1.0]hexane-2,6-dicarboxylic acid,
(1S*, 2S*, 4S*, 5R*, 6S*) 2-Amino-4-(3-methoxy-benzoylamino)-bicyclo[3.1.0]hexane-2,6-dicarboxylic acid,
(1S*, 2S*, 4S*, 5R*, 6S*) 2-Amino-4-(3-methoxy-benzoylamino)-bicyclo[3.1.0]hexane-2,6-dicarboxylic acid,
(1S*, 2S*, 4S*, 5R*, 6S*) 2-Amino-4-(3-methyl-benzoylamino)-bicyclo[3.1.0]hexane-2,6-dicarboxylic acid,
(1S*, 2S*, 4S*, 5R*, 6S*) 2-Amino-4-(3-trifluoromethyl-benzoylamino)-bicyclo[3.1.0]hexane-2,6-dicarboxylic acid,
(1S*, 2S*, 4S*, 5R*, 6S*) 2-Amino-4-(3-fluoro-benzoylamino)-bicyclo[3.1.0]hexane-2,6-dicarboxylic acid,
(1S*, 2S*, 4S*, 5R*, 6S*) 2-Amino-4-(3,4-dichloro-benzoylamino)-bicyclo[3.1.0]hexane-2,6-dicarboxylic acid,
(1S*, 2S*, 4S*, 5R*, 6S*) 2-Amino-4-(3,5-dichloro-benzoylamino)-bicyclo[3.1.0]hexane-2,6-dicarboxylic acid,
(1S*, 2S*, 4S*, 5R*, 6S*) 2-Amino-4-(2,4-dichloro-benzoylamino)-bicyclo[3.1.0]hexane-2,6-dicarboxylic acid,
(1S*, 2S*, 4S*, 5R*, 6S*) 2-Amino-4-(2,3-dichloro-benzoylamino)-bicyclo[3.1.0]hexane-2,6-dicarboxylic acid,
(1S*, 2S*, 4S*, 5R*, 6S*) 2-Amino-4-(2-fluoro-benzoylamino)-bicyclo[3.1.0]hexane-2,6-dicarboxylic acid,
(1S*, 2S*, 4S*, 5R*, 6S*) 2-Amino-4-(1H-indole-2-carbonyl)-amino]-bicyclo[3.1.0]hexane-2,6-dicarboxylic acid,
(1S*, 2S*, 4S*, 5R*, 6S*) 2-Amino-4-(4-hydroxy-benzoylamino)-bicyclo[3.1.0]hexane-2,6-dicarboxylic acid,
(1S*, 2S*, 4S*, 5R*, 6S*) 2-Amino-4-(3-hydroxy-benzoylamino)-bicyclo[3.1.0]hexane-2,6-dicarboxylic acid,
(1S*, 2S*, 4S*, 5R*, 6S*) 2-Amino-4-(2-hydroxy-benzoylamino)-bicyclo[3.1.0]hexane-2,6-dicarboxylic acid,
(1S*, 2S*, 4S*, 5R*, 6S*) 2-Amino-4-(biphenyl-4-carbonyl)-amino]-bicyclo[3.1.0]hexane-2,6-dicarboxylic acid,
(1S*, 2S*, 4S*, 5R*, 6S*) 2-Amino-4-[(1H-indole-3-carbonyl)-amino]-bicyclo[3.1.0]hexane-2,6-dicarboxylic acid,
(1S*, 2S*, 4S*, 5R*, 6S*) 2-Amino-4-[(isoquinoline-1-carbonyl)-amino]-bicyclo[3.1.0]hexane-2,6-dicarboxylic acid,
(1S*, 2S*, 4S*, 5R*, 6S*)-2-Amino-4-[(pyridine-2-carbonyl)-amino]-bicyclo[3.1.0]hexane-2,6-dicarboxylic acid,
(1S*, 2S*, 4S*, 5R*, 6S*)-2-Amino-4-[(pyridine-3-carbonyl)-amino]-bicyclo[3.1.0]hexane-2,6-dicarboxylic acid,
(1S*, 2S*, 4S*, 5R*, 6S*)-2-Amino-4-[(pyridine-4-carbonyl)-amino]-bicyclo[3.1.0]hexane-2,6-dicarboxylic acid,
(1S*, 2S*, 4S*, 5R*, 6S*)-2-Amino-4-(2-trifluoromethyl-benzoylamino)-bicyclo[3.1.0]hexane-2,6-dicarboxylic acid,
(1S*, 2S*, 4S*, 5R*, 6S*)-2-Amino-4-(3,5-difluoro-benzoylamino)-bicyclo[3.1.0]hexane-2,6-dicarboxylic acid,
(1S*, 2S*, 4S*, 5R*, 6S*)-2-Amino-4-(2-methyl-benzoylamino)bicyclo[3.1.0]hexane-2,6-dicarboxylic acid,
(1S*, 2S*, 4S*, 5R*, 6S*)-2-Amino-4-(4-methyl-benzoylamino)-bicyclo[3.1.0]hexane2,6-dicarboxylic acid,
(1S*, 2S*, 4S*, 5R*, 6S*)-2-Amino-4-[(quinoxaline-2-carbonyl)-amino]-bicyclo[3.1.0]hexane-2,6-dicarboxylic acid,
(1S*, 2S*, 4S*, 5R*, 6S*)-2-Amino-4-(3-carboxy-benzoylamino)-bicyclo[3.1.0]hexane-2,6-dicarboxylic acid,
(1S*, 2S*, 4S*, 5R*, 6S*)-2-Amino-4-(2-carboxy-benzoylamino)-bicyclo[3.1.0]hexane-2,6-dicarboxylic acid,
(1S*, 2S*, 4S*, 5R*, 6S*)-2-Amino-4-(4-carboxy-benzoylamino)-bicyclo[3.1.0]hexane-2,6-dicarboxylic acid,
(1S*, 2S*, 4S*, 5R*, 6S*)-2-Amino-4-[(1H-imidazol-4-carbonyl)-amino]-bicyclo[3.1.0]hexane-2,6-dicarboxylic acid,
(1S*, 2S*, 4S*, 5R*, 6S*)-2-Amino-4-(3-phenyl-acroloylamino)-bicyclo[3.1.0]hexane-2,6-dicarboxylic acid,
(1S*, 2S*, 4S*, 5R*, 6S*)-2-Amino-4-(3-phenyl-propynoylamino)-bicyclo[3.1.0]hexane-2,6-dicarboxylic acid,
(1S*, 2S*, 4S*, 5R*, 6S*)-2-Amino-4-benzylamino-bicyclo[3.1.0]hexane-2,6-dicarboxylic acid,
(1S*,2S*,4S*,5R*,6R*) 2-Amino-4-phenylcarbamoyloxy-bicyclo[3.1.0]hexane-2,6-dicarboxylic acid,
(1S*,2S*,4S*,5R*,6R*)-2-Amino-4(2-chloro-phenylcarbamoyloxy)-bicyclo[3.1.0]hexane-2,6-dicarboxylic acid,
(1S*,2S*,4S*,5R*,6R*)-2-Amino-4-(2-fluoro-phenylcarbamoyloxy)-bicyclo[3.1.0]hexane-2,6-dicarboxylic acid,
(1S*,2S*,4S*,5R*,6R*)-2-Amino-4-(3-fluoro-phenylcarbamoyloxy)-bicyclo[3.1.0]hexane-2,6-dicarboxylic acid,
(1S*,2S*,4S*,5R*,6R*)-2-Amino-4-(4-fluoro-phenylcarbamoyloxy)-bicyclo[3.1.0]hexane-2,6-dicarboxylic acid,
(1S*,2S*,4S*,5R*,6R*)-2-Amino-4-(3-chloro-phenylcarbamoyloxy)-bicyclo[3.1.0]hexane-2,6-dicarboxylic acid,
(1S*,2S*,4S*,5R*,6R*)-2-Amino-4-(4-chloro-phenylcarbamoyloxy)-bicyclo[3.1.0]hexane-2,6-dicarboxylic acid,
(1S*,2S*,4S*,5R*,6R*)-2-Amino-4-(naphthalen-1-ylcarbamoyloxy)-bicyclo[3.1.0]hexane-2,6-dicarboxylic acid,
(1S*,2S*,4S*,5R*,6R*)-2-Amino-4-(naphthalen-2-ylcarbamoyloxy)-bicyclo[3.1.0]hexane-2,6-dicarboxylic acid,
(1S*,2S*,4S*,5R*,6R*) 2-Amino-4-(2,4-dichlorophenoxy)bicyclo[3.1.0]hexane-2,6-dicarboxylic acid,
(1S*,2S*,4R*,5R*,6S*) 2-Amino-4-phenyl-carbamoylmethylbicyclo[3.1.0]hexane-2,6-dicarboxylic acid,
(1S*,2S*,4R*,5R*,6S*) 2-Amino-4-(o-methoxy)phenylcarbamoylmethyl bicyclo[3.1.0]hexane-2,6-dicarboxylic acid,
(1S*,2S*,4R*,5R*,6S*) 2-Amino-4-(o-methyl)phenylcarbamoylmethyl bicyclo[3.1.0]hexane-2,6-dicarboxylic acid,
(1S*,2S*,4R*,5R*,6S*) 2-Amino-4-(o-trifluoro methyl)phenylcarbamoylmethyl bicyclo[3.1.0]hexane-2,6-dicarboxylic acid,
(1S*,2S*,4R*,5R*,6S*) 2-Amino-4-(o-chloro)phenylcarbamoylmethyl bicyclo[3.1.0]hexane-2,6-dicarboxylic acid,
(1S*,2S*,4R*,5R*,6S*) 2-Amino-4-(m-methoxy)phenylcarbamoylmethyl bicyclo[3.1.0]hexane-2,6-dicarboxylic acid,
(1S*,2S*,4R*,5R*,6S*) 2-Amino-4-(m-methyl)phenylcarbamoylmethyl bicyclo[3.1.0]hexane-2,6-dicarboxylic acid,
(1S*,2S*,4R*,5R*,6S*) 2-Amino-4-(m-trifluoro methyl)phenylcarbamoylmethyl bicyclo[3.1.0]hexane-2,6-dicarboxylic acid,
(1S*,2S*,4R*,5R*,6S*) 2-Amino-4-(p-methoxy)phenylcarbamoylmethyl bicyclo[3.1.0]hexane2,6-dicarboxylic acid,
(1S*,2S*,4R*,5R*,6S*) 2-Amino-4-(p-methyl)phenylcarbamoylmethyl bicyclo[3.1.0]hexane-2,6-dicarboxylic acid,
(1S*,2S*,4R*,5R*,6S*) 2-Amino-4-(p-trifluoro methyl)Phenylcarbamoylmethyl bicyclo[3.1.0]hexane-2,6-dicarboxylic acid,
(1S*,2S*,4R*,5R*,6S*) 2-Amino-4-(m-chloro)phenylcarbamoylmethyl bicyclo[3.1.0]hexane-2,6-dicarboxylic acid,
(1S*,2S*,4R*,5R*,6S*) 2-Amino-4-carboxymethyl bicyclo[3.1.0]hexane-2,6-dicarboxylic acid,
(1S*,2S*,4R*,5R*,6S*) 2-Amino-4-benzoxazol-2-yl-methylbicyclo[3.1.0]hexane-2,6-dicarboxylic acid,
(1S*,2S*,4R*,5R*,6S*) 2-Amino-4-benzotiazol-2-yl-methylbicyclo[3.1.0]hexane-2,6-dicarboxylic acid, or
(1S*,2S*,4R*,5R*,6S*) 2-Amino-4-benzylcarbamoyl methylbicyclo[3.1.0]hexane-2,6-dicarboxylic acid.

10. A method for affecting the cAMP-linked metabotropic glutamate receptors in a patient, which comprises administering to a patient requiring modulated excitatory amino acid neurotransmission a pharmaceutically-effective amount of a compound of claim 9,

11. A method for treating a neurological disorder in a patient which comprises administering to the patient in need of treatment thereof a pharmaceutically-effective amount of a compound of claim 9.

12. The method of claim 11 wherein said neurological disorder is cerebral deficits subsequent to cardiac bypass and grafting; cerebral ischemia; spinal cord trauma; head trauma; Alzheimer's Disease; Huntington's Chorea; amyotrophic lateral sclerosis; AIDS-induced dementia; perinatal hypoxia; hypoglycemic neuronal damage; ocular damage and retinopathy; cognitive disorders; idiopathic and drug-induced Parkinson's Disease; muscular spasms; migraine headaches; urinary incontinence; drug tolerance, withdrawal, and cessation; smoking cessation; emesis; brain edema; chronic pain; sleep disorders; convulsions; Tourette's syndrome; attention deficit disorder; and tardive dyskinesia.

13. The method of claim 12 wherein said neurological disorder is drug tolerance, withdrawal, and cessation; or smoking cessation.

14. A method for treating a psychiatric disorder in a patient which comprises administering to the patient in need of treatment thereof a pharmaceutically-effective amount of a compound of claim 9.

15. The method of claim 14 wherein said psychiatric disorder is schizophrenia, anxiety and related disorders, depression, bipolar disorders, psychosis, and obsessive compulsive disorders.

16. The method of claim 15 in said psychiatric disorder is anxiety and related disorders.

17. A pharmaceutical formulation comprising a compound of claim 9 in combination with one or more pharmaceutically-acceptable carriers, diluents, or excipients.

18. A novel compound of formula I substantially as hereinbefore described with reference to any of the Examples.

19. A method for affecting the CAMP-linked metabotropic glutamate receptors in a patient, which comprises administering to a patient requiring modulated excitatory amino acid neurotransmission a pharmaceutically effective amount of a compound of formula I substantially as hereinbefore described with reference to any of the Examples.

20. A process for preparing a novel compound of formula I substantially as hereinbefore described with reference to any of the Examples.
